SEQUENCE LISTING (11 sequences)

SEQ ID NO: 1 - protein, 707 aa, Homo sapiens [sequence omitted]

SEQ ID NO: 2 - protein, 427 aa, Homo sapiens
MAPRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTQDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYGP

SEQ ID NO: 3 - protein, 417 aa, Homo sapiens
MVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTQDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYG

SEQ ID NO: 4 - protein, 242 aa, Homo sapiens
MVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGQGYSLFLVAAHEFGHALGLDHSSVPEALMYPMYRFTEGPPLHKDDVNGIRHLYG

SEQ ID NO: 5 - protein, 204 aa, Homo sapiens
MLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGQGYSLFLVAAHEFGHALGLDHSSVPEALMYPMYRFTEGPPLHKDDVNGIRHLYG

SEQ ID NO: 6 - protein, 251 aa, Homo sapiens
MAPRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGQGYSLFLVAAHEFGHALGLDHSSVPEALMYPMYRFTEGPPLHKDDVNGIRHLYG

SEQ ID NO: 7 - protein, 268 aa, Homo sapiens
MHPGVLAAFLFLSWTHCRALPLPSGGDEDDLSEEDLQFAERYLRSYYHPTNLAGILKENAASSMTERLREMQSFFGLEVTGKLDDNTLDVMKKPRCGVPDVGEYNVFPRTLKWSKMNLTYRIVNYTPDMTHSEVEKAFKKAFKVWSDVTPLNFTRLHDGIADIMISFGIKEHGDFYPFDGPSGLLAHAFPPGPNYGGDAHFDDDETWTSSSKGYNLFLVAAHEFGHSLGLDHSKDPGALMFPIYTYTGKSHFMLPDDDVQGIQSLYGP

SEQ ID NO: 8 - protein, 167 aa, Homo sapiens
MFRTFPGIPKWRKTHLTYRIVNYTPDLPKDAVDSAVEKALKVWEEVTPLTFSRLYEGEADIMISFAVREHGDFYPFDGPGNVLAHAYAPGPGINGDAHFDDDEQWTKDTTGTNLFLVAAHEIGHSLGLFHSANTEALMYPLYHSLTDLTRFRLSQDDINGIQSLYGP

SEQ ID NO: 9 - protein, 7 aa, Homo sapiens
FQTFEGD

SEQ ID NO: 10 - protein, 15 aa, Homo sapiens
CGVPDLGRFQTFEGD

SEQ ID NO: 11 - protein, 708 aa, Rattus norvegicus
MSPWQPLLLVLLALGYSFAAPHQRQPTYVVFPRDLKTSNLTDTQLAEDYLYRYGYTRAAQMMGEKQSLRPALLMLQKQLSLPQTGELDSETLKAIRSPRCGVPDVGKFQTFDGDLKWHHHNITYWIQSYTEDLPRDVIDDSFARAFAVWSAVTPLTFTRVYGLEADIVIQFGVAEHGDGYPFDGKDGLLAHAFPPGPGIQGDAHFDDDELWSLGKGAVVPTYFGNANGAPCHFPFTFEGRSYLSCTTDGRNDGKPWCGTTADYDTDRKYGFCPSENLYTEHGNGDGKPCVFPFIFEGHSYSACTTKGRSDGYRWCATTANYDQDKADGFCPTRADVTVTGGNSAGEMCVFPFVFLGKQYSTCTSEGRSDGRLWCATTSNFDADKKWGFCPDQGYSLFLVAAHEFGHALGLDHSSVPEALMYPMYHYHEDSPLHEDDIKGIHHLYGRGSKPDPRPPATTAAEPQPTAPPTMCSTAPPMAYPTGGPTVAPTGAPSPGPTGPPTAGPSEAPTESSTPDDNPCNVDVFDAIADIQGALHFFKDGRYWKFSNHGGNQLQGPFLIARTWPAFPSKLNSAFEDPQPKKIFFFLWAQMWVYTGQSVLGPRSLDKLGLGSEVTLVTGLLPRRGGKALLISRERIWKFDLKSQKVDPQSVTRLDNEFSGVPWNSHNVFQYQDKAYFCHDKYFWRVSFHNRVNQVDHVAYVTYDLLQCP

(19) United States
(12) Patent Application Publication
(10) Pub. No.: US 2012/0302573 A1
(43) Pub. Date: Nov. 29, 2012

(54) METHODS OF INHIBITING PRO MATRIX METALLOPROTEINASE ACTIVATION

(76) Inventors: Paul Francis JACKSON, New Hope, PA (US); Carl Manthey, Chester Springs, PA (US); Kenneth Rhodes, Hopkinton, MA (US); Robert Scannevin, Hopkinton, MA (US); Kristi Anne Leonard, Lansdale, PA (US); Joseph Kent Barbay, Flourtown, PA (US); Matthew Todd, Ambler, PA (US); Barry A. Springer, Wilmington, DE (US)

(21) Appl. No.: 13/479,421

(22) Filed: May 24, 2012

Related U.S. Application Data
(60) Provisional application No. 61/489,733, filed on May 25, 2011.

(51) Int. Cl. C12N 9/99; A61K 31/4439; A61K 31/497; A61K 31/437; A61K 31/519; A61K 31/444; A61K 31/4985; A61P 35/00; A61P 19/02; A61P 9/00; A61P 1/04; A61P 9/12; A61P 11/00; A61P 1/00; A61P 1/02; A61P 17/02; A61P 25/06; A61K 31/427

(52) U.S. Cl. 514/249; 435/184; 514/371; 514/370; 514/342; 514/255.05; 514/300; 514/259.1; 514/333

(57) ABSTRACT

This invention relates to methods for preventing, treating or ameliorating an MMP9 and/or MMP13 mediated syndrome, disorder or disease comprising administering to a subject in need thereof an effective amount of a compound listed in the examples section of this specification, or a form, composition or medicament thereof. Disorders treated and/or prevented include rheumatoid arthritis.

### CROSS-REFERENCE TO RELATED APPLICATIONS

The present application claims the benefits of the filing of U.S. Provisional Application No. 61/489,733 filed May 25, 2011. The complete disclosures of the aforementioned related patent applications are hereby incorporated herein by reference for all purposes.

### TECHNICAL FIELD

The present invention relates to methods of inhibiting pro-matrix metalloproteinase activation and associated therapeutic and prophylactic applications. Disorders treated and/or prevented include inflammation related disorders and disorders ameliorated by inhibiting the proteolytic activation of pro-matrix metalloproteinases.

### BACKGROUND OF THE INVENTION

Matrix metalloproteinases (MMPs) are a family of structurally related zinc-dependent proteolytic enzymes that digest extracellular matrix proteins such as collagen, elastin, laminin and fibronectin. Currently, at least 28 different mammalian MMP proteins have been identified and they are grouped based on substrate specificity and domain structure. Enzymatic activities of the MMPs are precisely controlled, not only by their gene expression in various cell types, but also by activation of their inactive zymogen precursors (proMMPs) and inhibition by endogenous inhibitors and tissue inhibitors of metalloproteinases (TIMPs). The enzymes play a key role in normal homeostatic tissue remodeling events, but are also considered to play a key role in pathological destruction of the matrix in many connective tissue diseases such as arthritis, periodontitis, and tissue ulceration and also in cancer cell invasion and metastasis.

A role for MMPs in oncology is well established, as up-regulation of any number of MMPs are one mechanism by which malignant cells can overcome connective tissue barriers and metastasize (Curr Cancer Drug Targets 5(3): 203-20, 2005). MMPs also appear to have a direct role in angiogenesis, which is another reason they have been an important target for oncology indications (Int J Cancer 115(6): 849-60, 2005; J Cell Mol Med 9(2): 267-85, 2005). Several different classes of MMPs are involved in these processes, including MMP9.

Other MMP mediated indications include the cartilage and bone degeneration that results in osteoarthritis and rheumatoid arthritis. The degeneration is due primarily to MMP digestion of the extracellular matrix (ECM) in bone and joints (Aging Clin Exp Res 15(5): 364-72, 2003). Various MMPs, including MMP9 and MMP13 have been found to be elevated in the tissues and body fluids surrounding the damaged areas.

Elevated MMP levels, including MMP9 and MMP13 are also believed to be involved in atherosclerotic plaque rupture, aneurysm and vascular and myocardial tissue morphogenesis (Expert Opin Investig Drugs 9(5): 993-1007, 2000; Curr Med Chem 12(8): 917-25, 2005). Elevated levels of MMPs, including MMP9 and MMP13, have often been associated with these conditions. Several other pathologies such as gastric ulcers, pulmonary hypertension, chronic obstructive pulmonary disease, inflammatory bowel disease, periodontal disease, skin ulcers, liver fibrosis, emphysema, and Marfan syndrome all appear to have an MMP component as well (Expert Opinion on Therapeutic Patents 12(5): 665-707, 2002).

Within the central nervous system, altered MMP expression has been linked to several neurodegenerative disease states (Expert Opin Investig Drugs 8(3): 255-68, 1999), most notably in stroke (Glia 50(4): 329-39, 2005). MMPs, including MMP9, have been shown to have an impact in propagating the brain tissue damage that occurs following an ischemic or hemorrhagic insult. Studies in human stroke patients and in animal stroke models have demonstrated that expression levels and activity of MMPs, including MMP9, increase sharply over a 24 hour period following an ischemic event. Administration of MMP inhibitors has been shown to be protective in animal models of stroke (Expert Opin Investig Drugs 8(3): 255-68, 1999; J Neurosci 25(27): 6401-8, 2005). In addition, MMP9 knockout animals also demonstrate significant neuroprotection in similar stroke models (J Cereb Blood Flow Metab 20(12): 1681-9, 2000). In the US, stroke is the third leading cause of mortality, and the leading cause of disability. Thus stroke comprises a large unmet medical need for acute interventional therapy that could potentially be addressed with MMP inhibitors.

It has also been suggested that MMP9 may play a role in the progression of multiple sclerosis (MS). Studies have indicated that serum levels of MMP9 are elevated in active patients, and are concentrated around MS lesions (Lancet Neurol 2(12): 747-56, 2003). Increased serum MMP9 activity would promote infiltration of leukocytes into the CNS, a causal factor and one of the hallmarks of the disease. MMPs may also contribute to severity and prolongation of migraines. In animal models of migraine (cortical spreading depression), MMP9 is rapidly upregulated and activated leading to a breakdown in the BBB, which results in mild to moderate edema (J Clin Invest 113(10): 1447-55, 2004). It is this brain swelling and subsequent vasoconstriction which causes the debilitating headaches and other symptoms associated with migraine. In the cortical spreading depression model, MMP inhibitors have been shown to prevent the opening of the BBB (J Clin Invest 113(10): 1447-55, 2004). Related research has shown that MMP9 is specifically upregulated in damaged brain tissues following traumatic brain injury (J Neurotrauma 19(5): 615-25, 2002), which would be predicted to lead to further brain damage due to edema and immune cell infiltration. MMPs may also have additional roles in additional chronic CNS disorders. In an animal model of Parkinson's disease, MMP9 was found to be rapidly upregulated after striatal injection of a dopaminergic neuron poison (MPTP).

With regard to structure and activation of the inactive zymogen form, a prototypical MMP is matrix metalloproteinase 9 (MMP9). MMP9 is also known as macrophage gelatinase, gelatinase B, 92 kDa gelatinase, 92 kDa type IV collagenase, and type V collagenase. The inactive form of MMP9, proMMP9, is expressed with several different domains including a signal sequence for secretion, a propeptide domain which inhibits activity of proMMP9, a catalytic domain for protein cleavage, a fibronectin type-II (FnII) domain consisting of three fibronectin-type II repeats, and a hemopexin-like domain thought to assist in substrate docking. The hemopexin-like domain also serves as a binding domain for interaction with tissue inhibitors of metalloproteinases (TIMPs). The inactive zymogen form of MMP9, proMMP9, is maintained through a cysteine-switch mechanism, in which a Cys in the propeptide forms a complex with the catalytic zinc in the catalytic domain and occludes the active site (Proc Natl Acad Sci USA 87(14): 5578-82, 1990). Activation of proMMP9 occurs in a two-step process. A protease cleaves an initial site after Met60, disrupting the zinc coordination and destabilizing the propeptide interaction with the catalytic domain. This initial cleavage allows access to the second cleavage site at Phe107, after which the propeptide is removed and the mature active form of the enzyme is released (Biol Chem 378(3-4): 151-60, 1997). The identity of the proMMP9 activating proteases is unknown in vivo, although there is evidence that activation can occur through the actions of MMP3, chymase and trypsin (J Biol Chem 267(6): 3581-4, 1992; J Biol Chem 272(41): 25628-35, 1997; J Biol Chem 280(10): 9291-6, 2005).

Based on the demonstrated involvement in numerous pathological conditions, inhibitors of matrix metalloproteases (MMPs) have therapeutic potential in a range of disease states. However, non-selective active site MMP inhibitors have performed poorly in clinical trials. The failures have often been caused by dose-limiting toxicity and the manifestation of significant side effects, including the development of musculoskeletal syndrome (MSS). It has been suggested that development of more selective MMP inhibitors might help to overcome some of the problems that hindered clinical success in the past, but there are a number of obstacles to developing more selective MMP active site inhibitors. MMPs share a catalytically important Zn2+ ion in the active site and a highly conserved zinc-binding motif. In addition, there is considerable sequence conservation across the entire catalytic domain for members of the MMP family.

A novel approach to developing more selective MMP inhibitors is to target the pro domain of the inactive zymogens, proMMPs, with allosteric small-molecule inhibitors that bind and stabilize the inactive pro form of the protein and inhibit processing to the active enzyme. There is significantly less sequence identity within the pro domains of MMP proteins, no catalytically important Zn2+ ion, and no highly conserved zinc-binding motif. Thus targeting the pro domain of proMMPs is an attractive mechanism of action for inhibiting the activity of the MMP proteins. Inhibition of proMMP9 activation has been observed with a specific monoclonal antibody (Hybridoma 12(4): 349-63, 1993). The activation of proMMP9 by trypsin has also been shown to be inhibited by Bowman-Birk inhibitor proteins and derived peptide inhibitors (Biotechnol Lett 26(11): 901-5, 2004). There are no reports, however, of allosteric small-molecule inhibitors that bind the pro domain and inhibit activation of proMMP9, proMMP13, or any other proMMP. The present invention provides methods of using small-molecules to allosterically inhibit the proteolytic activation of proMMP9, proMMP13, and methods of treatment.

### SUMMARY OF THE INVENTION

The invention comprises a method of inhibiting activation of matrix metalloproteinase proMMP9 and/or proMMP13 using a compound selected from the group consisting of:

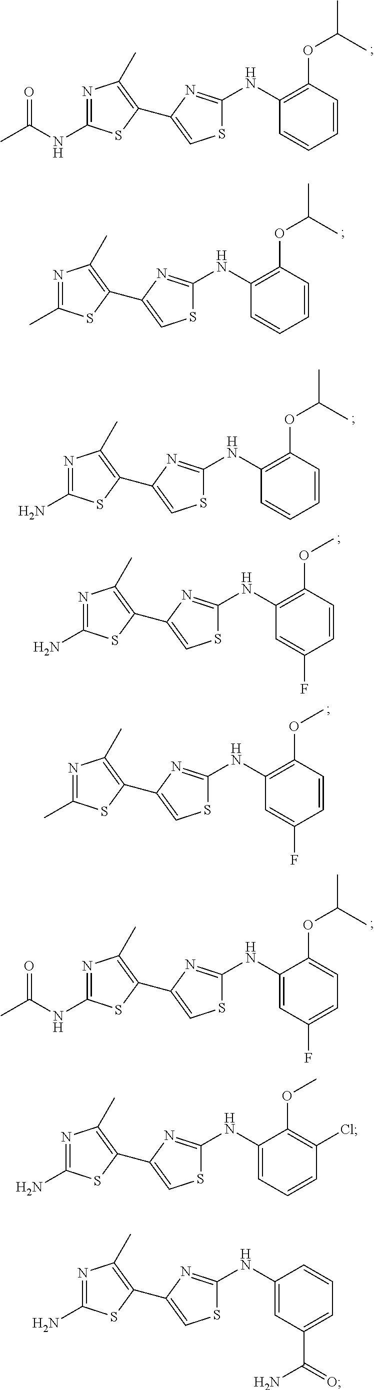

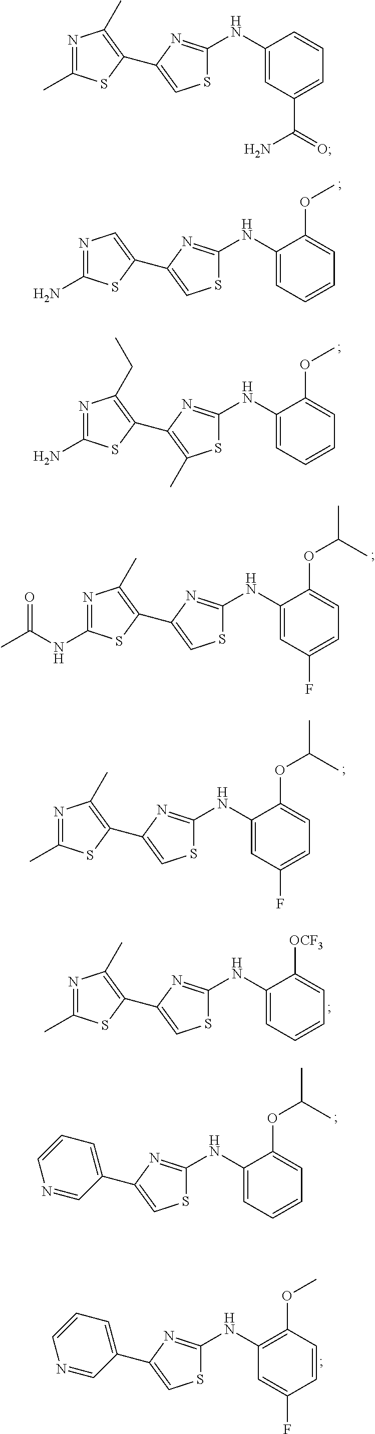

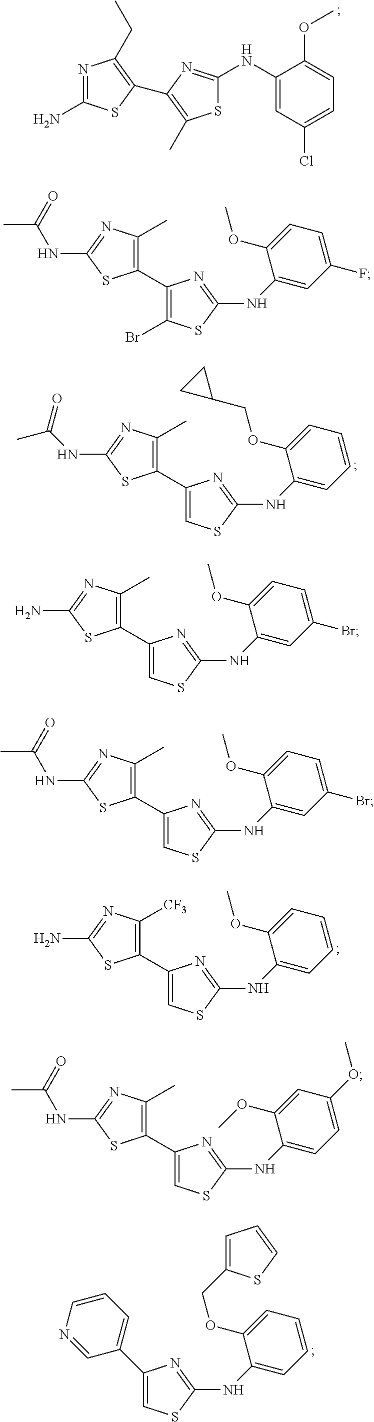

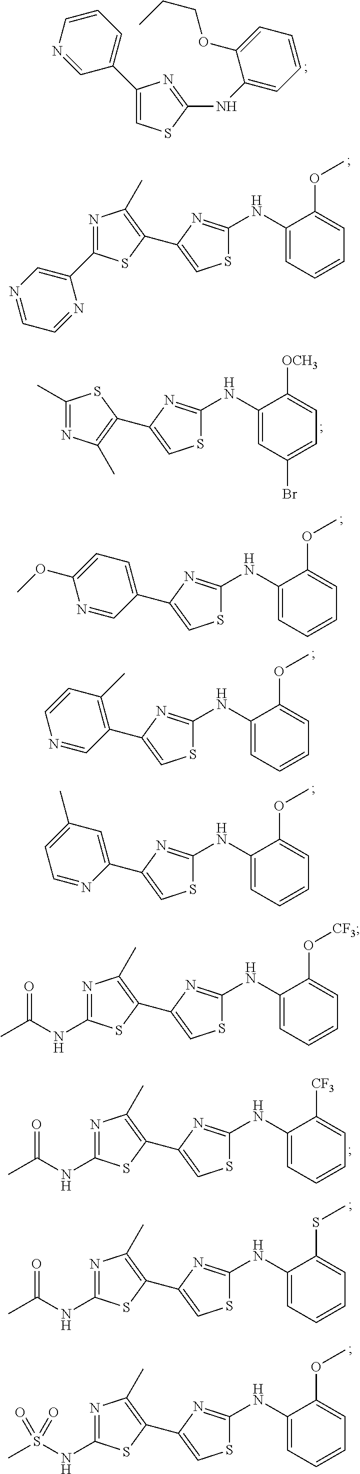

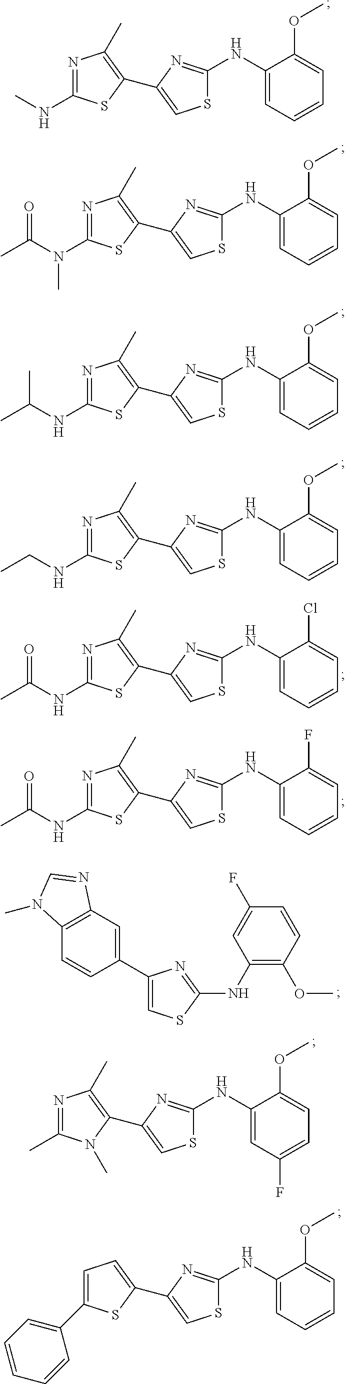

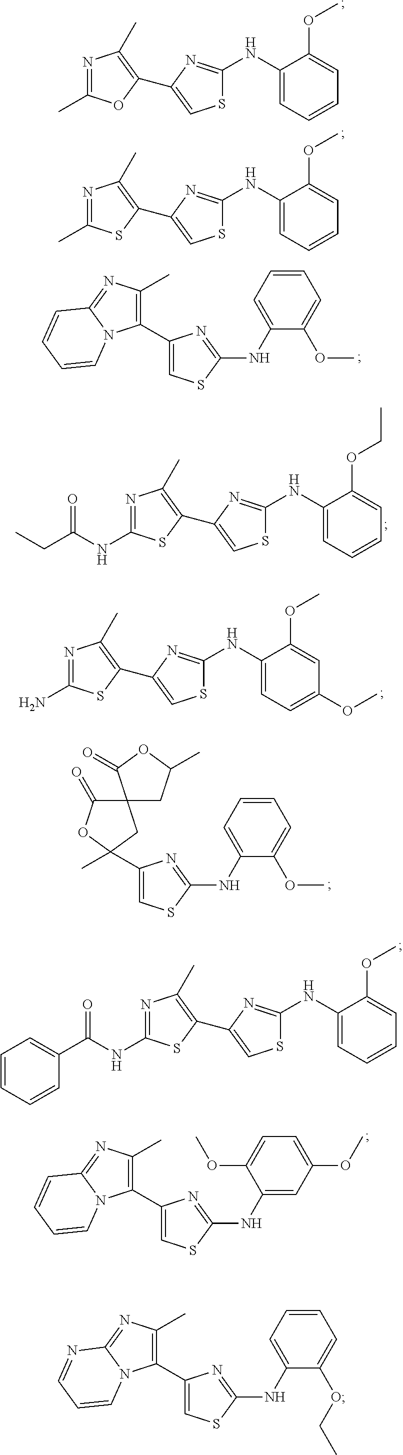

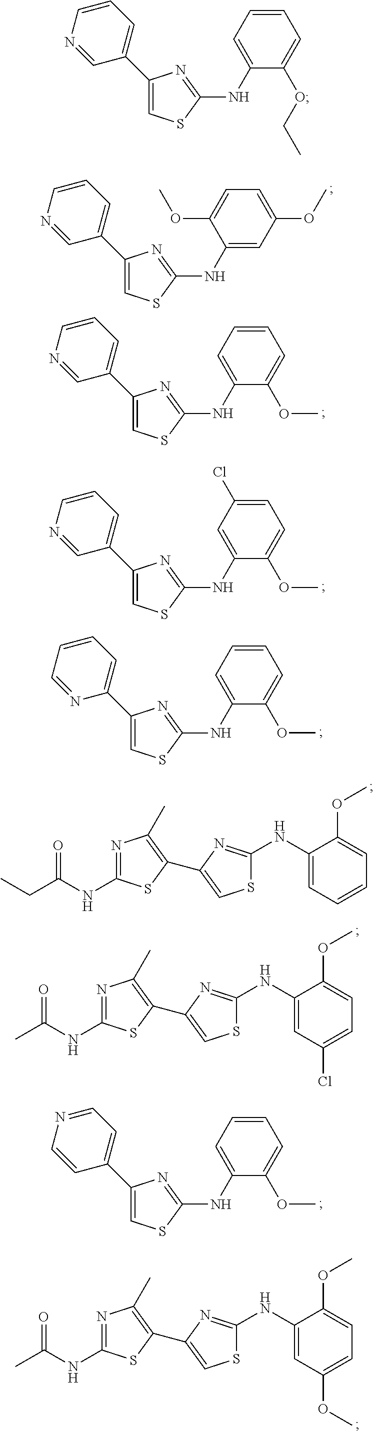

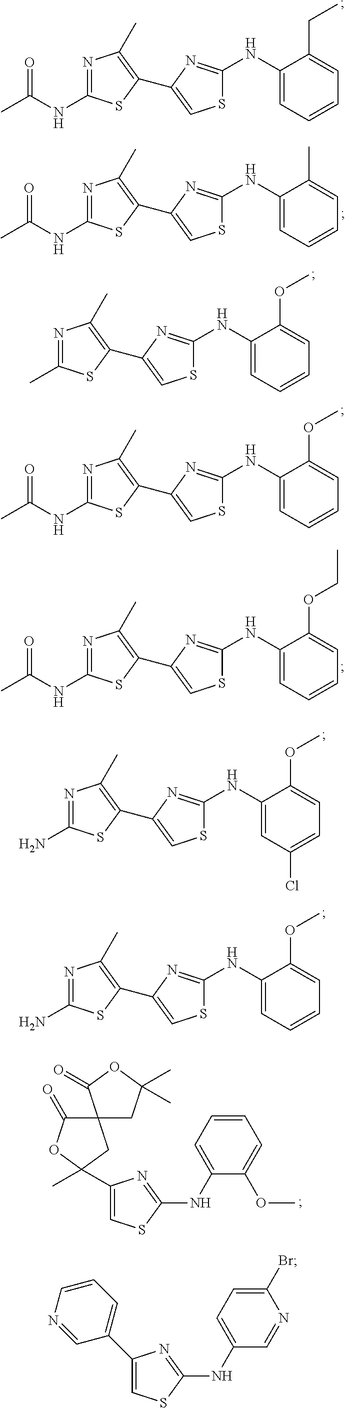

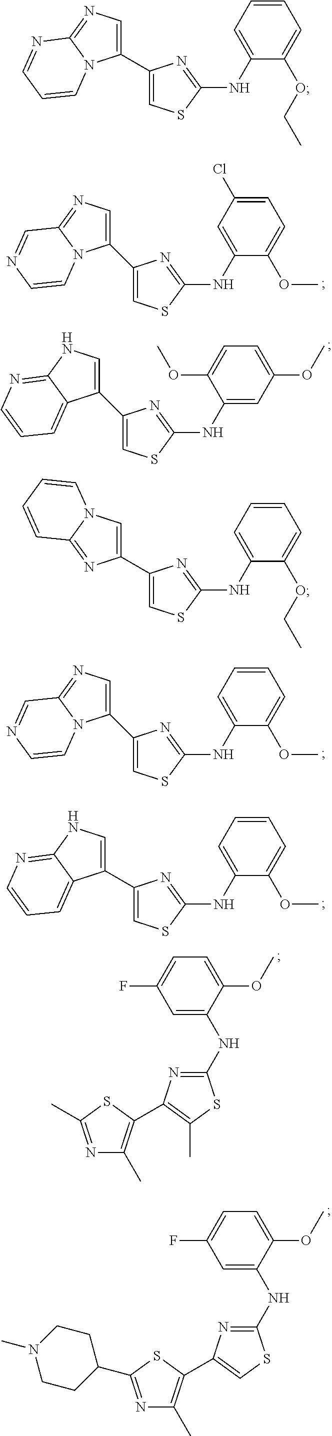

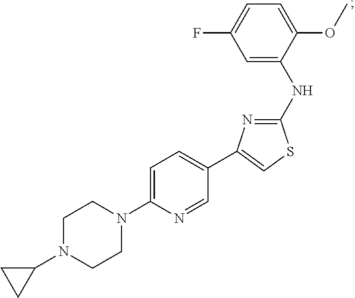

and solvates, hydrates, tautomers, and pharmaceutically acceptable salts thereof.

### BRIEF DESCRIPTION OF THE DRAWINGS

Embodiments of the present invention will now be described, by way of an example only, with reference to the accompanying drawings wherein:

FIG. 1: Shown are western blots with two different antibodies illustrating the effects of a small molecule allosteric processing inhibitor, Compound α, on the activation of proMMP9 in synoviocytes harvested from female Lewis rats after inducing arthritis with i.p. administration of Streptococcal cell wall peptidoglycan polysaccharides. A mouse monoclonal antibody, mAb L51/82, detected pro and processed forms of MMP9. The mouse monoclonal antibody showed that Compound α caused a dose-dependent reduction in the appearance of the 80 kD active form of MMP9 and the appearance of an 86 kD form of the protein (FIG. 1A, lanes 3-6). A rabbit polyclonal antibody, pAb-1246, detected the 80 kD active form of MMP9, but did not recognize the 100 kD form of proMMP9. The rabbit polyclonal antibody showed that the small molecule allosteric processing inhibitor caused a dose-dependent reduction in the appearance of the 80 kD active form of MMP9 (FIG. 1B, lanes 2-6).

FIG. 2: Shown are western blots illustrating increased proMMP9 and increased active MMP9 in tibia-tarsus joints (ankles) from female Lewis rats after inducing arthritis with i.p. administration of Streptococcal cell wall peptidoglycan polysaccharides (SCW). In healthy ankles of rats administered saline, mAb-L51/82 detected small amounts of an approximately 100 kD proMMP9 and an approximately 80 kD form of active MMP9 (FIG. 2A, lanes 1 and 2). The amount of proMMP9 increased markedly in ankle homogenates 5 and 18 days after SCW-administration (FIG. 2A, lanes 3-5 and 6-8, respectively). The amount of active 80 kD MMP9 increased mildly 5 days after SCW-administration (FIG. 2A, lanes 3-5) and increased markedly 18 days after SCW-administration (FIG. 2A, lanes 6-8). In healthy ankles of rats administered saline, mAb-1246 detected small amounts active 80 kD MMP9 (FIG. 2B, lanes 1 and 2). The 80 kD active MMP9 increased mildly 5 days after SCW-administration (FIG. 2A, lanes 3-5) and increased markedly 18 days after SCW-administration (FIG. 2A, lanes 6-8).

FIG. 3: Shown are western blots with two different antibodies illustrating the effects of a small molecule allosteric processing inhibitor, Compound α, on the activation of proMMP9 in tibia-tarsus joints (ankles) from female Lewis rats after inducing arthritis with i.p. administration of Streptococcal cell wall peptidoglycan polysaccharides (SCW). Both proMMP9 and active MMP9 were abundantly present in ankles of SCW-induced vehicle-treated rats (FIGS. 3A and 3B, lanes 1-3). Treatment of rats with Compound α did not reduce the abundance of proMMP-9 (FIG. 3A, lanes 4-9). However, treatment of rats with Compound α resulted in a notable reduction in the active 80 kD form of MMP9 detected with pAb-1246 (FIG. 3B, lanes 4-9) and also with mAb-L51/82 (FIG. 3A, lanes 4-9).

### DETAILED DESCRIPTION OF THE INVENTION

The invention comprises a method of inhibiting activation of matrix metalloproteinase proMMP9 and/or proMMP13 using a compound selected from the group consisting of:

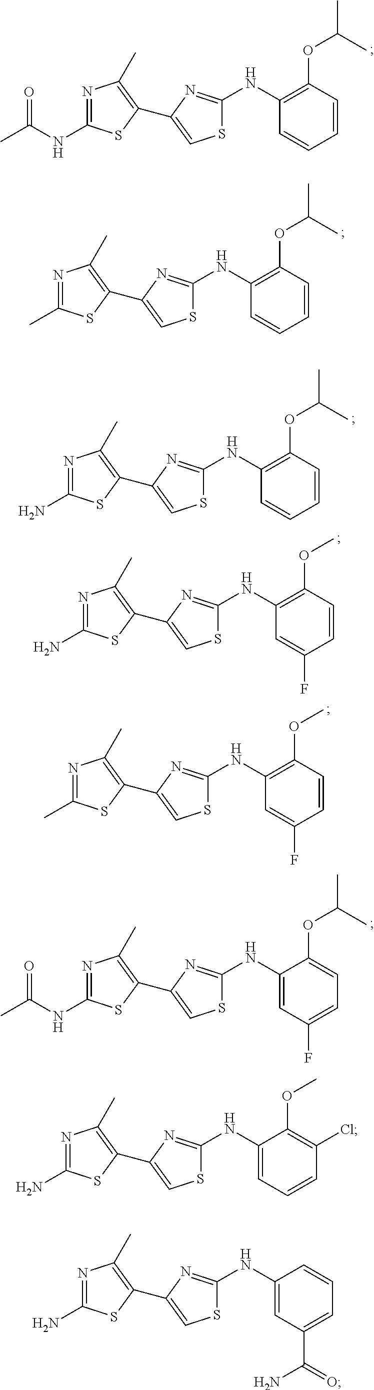

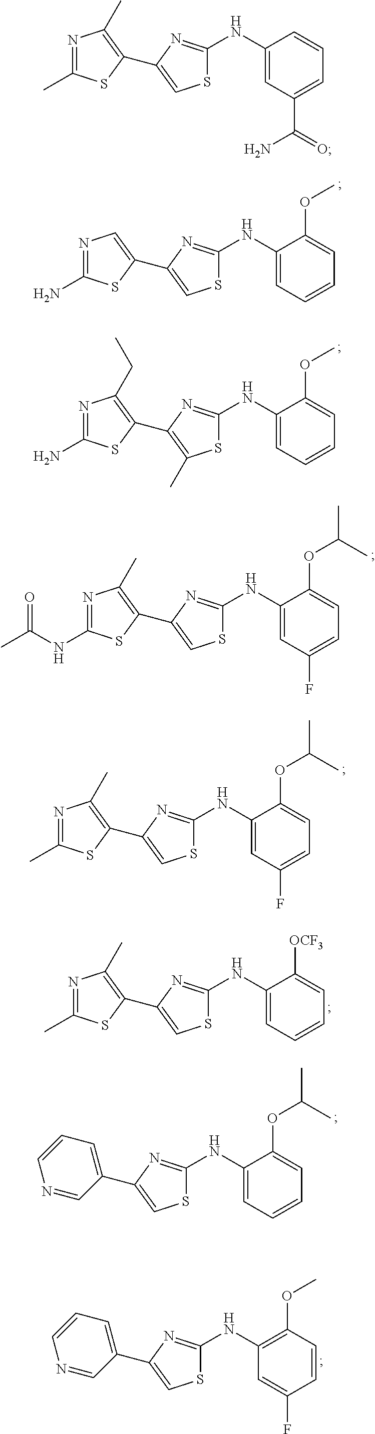

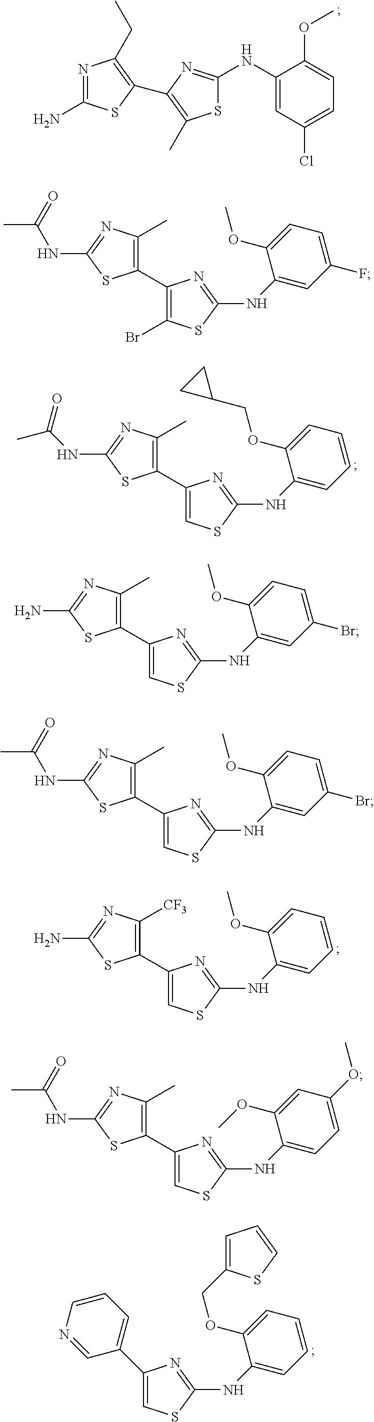

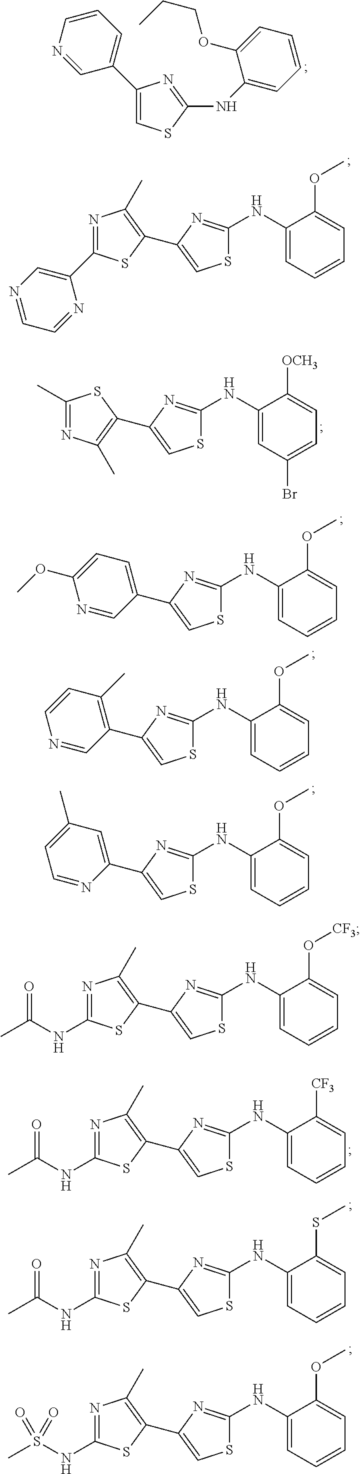

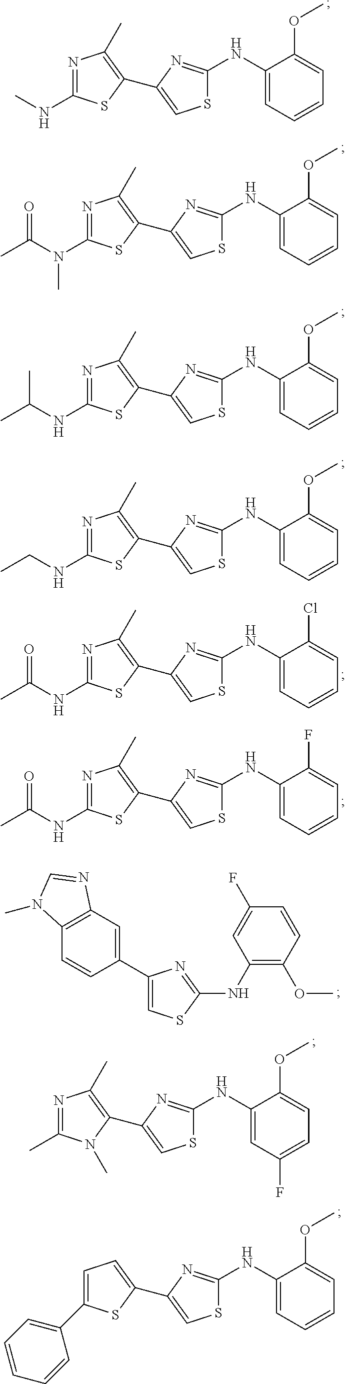

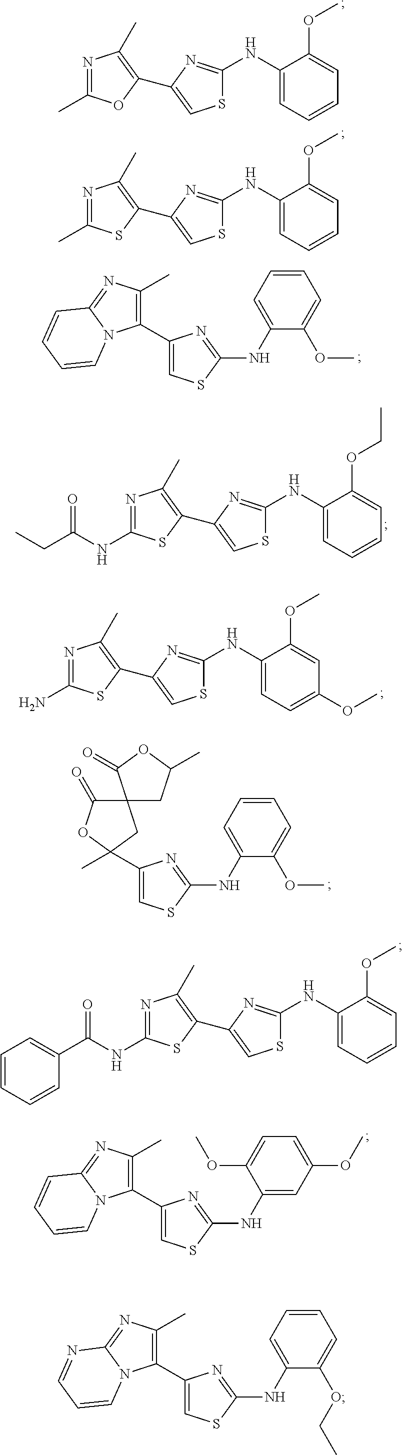

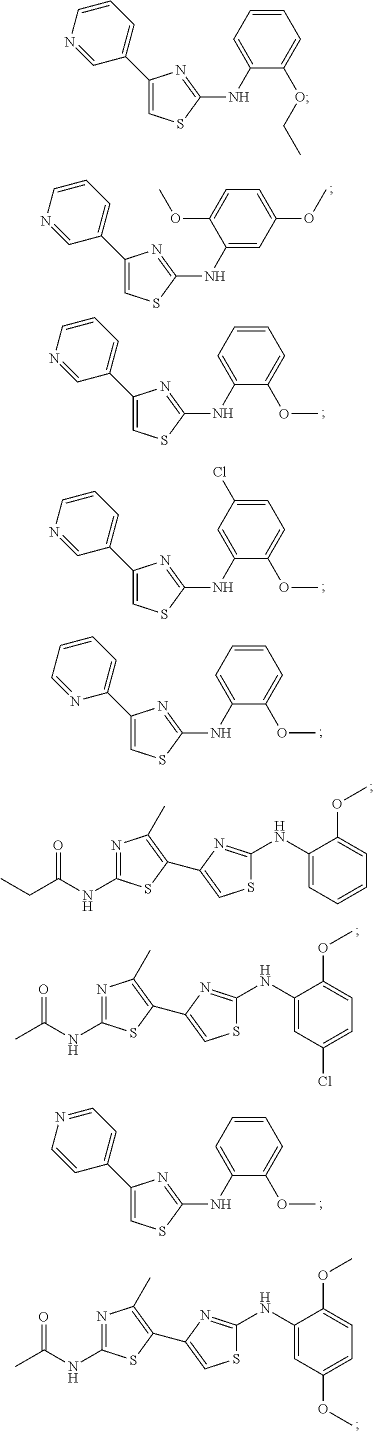

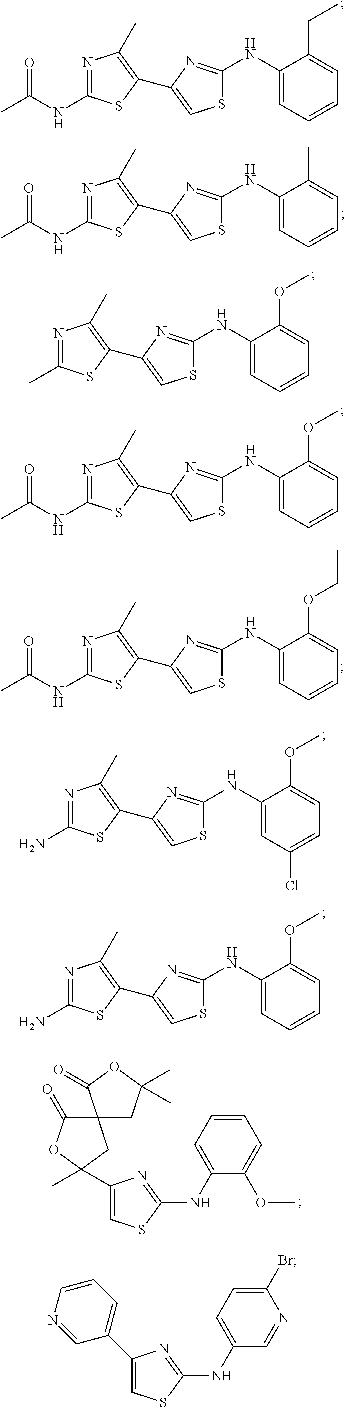

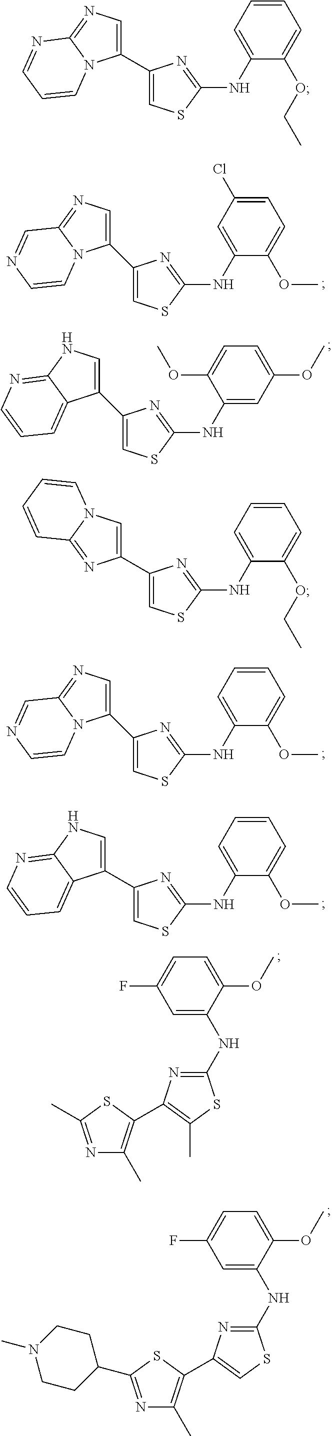

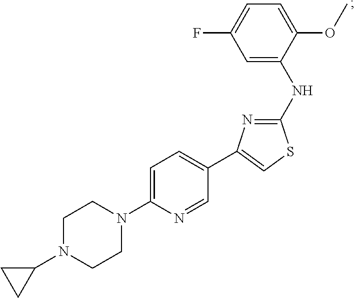

and solvates, hydrates, tautomers, and pharmaceutically acceptable salts thereof.

Another embodiment of the invention is a method of inhibiting activation of matrix metalloproteinase proMMP9 and/or proMMP13 using a pharmaceutical composition, comprising a compound listed in the examples section of this specification and a pharmaceutically acceptable carrier.

The present invention also provides a method for preventing, treating or ameliorating an MMP9 mediated syndrome, disorder or disease comprising administering to a subject in need thereof an effective amount of a compound listed in the examples section of this specification or a form, composition or medicament thereof.

The present invention also provides a method for preventing, treating or ameliorating an MMP13 mediated syndrome, disorder or disease comprising administering to a subject in need thereof an effective amount of a compound listed in the examples section of this specification or a form, composition or medicament thereof.

The present invention also provides a method for preventing, treating or ameliorating an MMP9 mediated syndrome, disorder or disease wherein said syndrome, disorder or disease is associated with elevated MMP9 expression or MMP9 overexpression, or is a condition that accompanies syndromes, disorders or diseases associated with elevated MMP9 expression or MMP9 overexpression comprising administering to a subject in need thereof an effective amount of a compound listed in the examples section of this specification or a form, composition or medicament thereof.

The present invention also provides a method for preventing, treating or ameliorating an MMP13 mediated syndrome, disorder or disease wherein said syndrome, disorder or disease is associated with elevated MMP13 expression or MMP13 overexpression, or is a condition that accompanies syndromes, disorders or diseases associated with elevated MMP13 expression or MMP13 overexpression comprising administering to a subject in need thereof an effective amount of a compound listed in the examples section of this specification or a form, composition or medicament thereof.

The present invention provides a method of preventing, treating or ameliorating a syndrome, disorder or disease, wherein said syndrome, disorder or disease is selected from the group consisting of: neoplastic disorders, osteoarthritis, rheumatoid arthritis, cardiovascular diseases, gastric ulcer, pulmonary hypertension, chronic obstructive pulmonary disease, inflammatory bowel syndrome, periodontal disease, skin ulcers, liver fibrosis, emphysema, Marfan syndrome, stroke, multiple sclerosis, asthma, abdominal aortic aneurysm, coronary artery disease, idiopathic pulmonary fibrosis, renal fibrosis, and migraine, comprising administering to a subject in need thereof an effective amount of a compound listed in the examples section of this specification or a form, composition or medicament thereof.

The present invention provides a method of preventing, treating or ameliorating a neoplastic disorder, wherein said neoplastic disorder is ovarian cancer, comprising administering to a subject in need thereof an effective amount of a compound listed in the examples section of this specification or a form, composition or medicament thereof.

The present invention provides a method of preventing, treating or ameliorating a cardiovascular disease, wherein said cardiovascular disease is selected from the group consisting of: atherosclerotic plaque rupture, aneurysm, vascular tissue morphogenesis, coronary artery disease, and myocardial tissue morphogenesis, comprising administering to a subject in need thereof an effective amount of a compound listed in the examples section of this specification or a form, composition or medicament thereof.

The present invention provides a method of preventing, treating or ameliorating atherosclerotic plaque rupture, comprising administering to a subject in need thereof an effective amount of a compound listed in the examples section of this specification or a form, composition or medicament thereof.

The present invention provides a method of preventing, treating or ameliorating rheumatoid arthritis, comprising administering to a subject in need thereof an effective amount of a compound listed in the examples section of this specification or a form, composition or medicament thereof.

The present invention provides a method of preventing, treating or ameliorating asthma, comprising administering to a subject in need thereof an effective amount of a compound listed in the examples section of this specification or a form, composition or medicament thereof.

The present invention provides a method of preventing, treating or ameliorating chronic obstructive pulmonary disease, comprising administering to a subject in need thereof an effective amount of a compound listed in the examples section of this specification or a form, composition or medicament thereof.

The present invention provides a method of preventing, treating or ameliorating inflammatory bowel syndrome, comprising administering to a subject in need thereof an effective amount of a compound listed in the examples section of this specification or a form, composition or medicament thereof.

The present invention provides a method of preventing, treating or ameliorating abdominal aortic aneurism, comprising administering to a subject in need thereof an effective amount of a compound listed in the examples section of this specification or a form, composition or medicament thereof.

The present invention provides a method of preventing, treating or ameliorating osteoarthritis, comprising administering to a subject in need thereof an effective amount of a compound listed in the examples section of this specification or a form, composition or medicament thereof.

The present invention provides a method of preventing, treating or ameliorating idiopathic pulmonary fibrosis, comprising administering to a subject in need thereof an effective amount of a compound listed in the examples section of this specification or a form, composition or medicament thereof.

The invention also relates to methods of inhibiting MMP9 activity in a mammal by administration of an effective amount of at least one compound listed in the examples section of this specification.

The invention also relates to methods of inhibiting MMP13 activity in a mammal by administration of an effective amount of at least one compound listed in the examples section of this specification.

In another embodiment, the invention relates to a compound as described in the Examples section for use as a medicament, in particular, for use as a medicament for treating a MMP9 mediated syndrome, disorder or disease.

In another embodiment, the invention relates to the use of a compound as described in the Examples section for the preparation of a medicament for the treatment of a disease associated with an elevated or inappropriate MMP9 activity.

In another embodiment, the invention relates to a compound as described in the Examples section for use as a medicament, in particular, for use as a medicament for treating a MMP13 mediated syndrome, disorder or disease.

In another embodiment, the invention relates to the use of a compound as described in the Examples section for the preparation of a medicament for the treatment of a disease associated with an elevated or inappropriate MMP13 activity.

### DEFINITIONS

The term “alkyl” refers to both linear and branched chain radicals of up to 12 carbon atoms, preferably up to 6 carbon atoms, unless otherwise indicated, and includes, but is not limited to, methyl, ethyl, propyl, isopropyl, butyl, isobutyl, sec-butyl, tert-butyl, pentyl, isopentyl, hexyl, isohexyl, heptyl, octyl, 2,2,4-trimethylpentyl, nonyl, decyl, undecyl and dodecyl. Any alkyl group may be optionally substituted with one OCH₃, one OH, or up to two fluorine atoms.

The term “alkoxy” refers to a saturated branched or straight chain monovalent hydrocarbon alcohol radical derived by the removal of the hydrogen atom from the hydroxide oxygen substituent on a parent alkane. Examples include C₍₁₋₆₎alkoxy or C₍₁₋₄₎alkoxy groups. Any alkoxy group may be optionally substituted with one OCH₃, one OH, or up to two fluorine atoms.

The term “C_{(a-b)}” (where a and b are integers referring to a designated number of carbon atoms) refers to an alkyl, alkenyl, alkynyl, alkoxy or cycloalkyl radical or to the alkyl portion of a radical in which alkyl appears as the prefix root containing from a to b carbon atoms inclusive. For example, C₍₁₋₄₎ denotes a radical containing 1, 2, 3 or 4 carbon atoms.

The term “cycloalkyl” refers to a saturated or partially unsaturated monocyclic or bicyclic hydrocarbon ring radical derived by the removal of one hydrogen atom from a single ring carbon atom. Typical cycloalkyl radicals include cyclopropyl, cyclobutyl, cyclopentyl, cyclopentenyl, cyclohexyl, cyclohexenyl, cycloheptyl and cyclooctyl. Additional examples include C₍₃₋₆₎cycloalkyl, C₍₅₋₈₎cycloalkyl, decahydronaphthalenyl, and 2,3,4,5,6,7-hexahydro-1H-indenyl. Any cycloalkyl group may be optionally substituted with one OCH₃, one OH, or up to two fluorine atoms.

### ABBREVIATIONS

Herein and throughout this application, the following abbreviations may be used.

### Ac —C(O)CH₃

aq. aqueous
conc. concentrated
DCM dichloromethane
DIAD diisopropyl azodicarboxylate
DMAP dimethylaminopyridine
DMSO dimethylsulfoxide
Et ethyl
D deuterium
d days
g gram
h hours
hept heptanes
HPLC high pressure liquid chromatography
M molar
Me methyl
mL milliliter
mmol millimole
mg milligram
min minutes
N normal
NMR nuclear magnetic resonance
iPr isopropyl
RP-HPLC reverse phase high pressure liquid chromatography
RT or rt room temperature
sat. saturated
TFA trifluoroacetic acid
THF tetrahydrofuran
TLC thin layer chromatography
UV ultra violet
v volume
W watts

Pharmaceutically acceptable acidic/anionic salts include, and are not limited to acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, methylbromide, methylnitrate, methylsulfate, mucate, napsylate, nitrate, pamoate, pantothenate, phosphate/diphosphate, polygalacturonate, salicylate, stearate, subacetate, succinate, sulfate, tannate, tartrate, teoclate, tosylate and triethiodide. Organic or inorganic acids also include, and are not limited to, hydriodic, perchloric, sulfuric, phosphoric, propionic, glycolic, methanesulfonic, hydroxyethanesulfonic, oxalic, 2-naphthalenesulfonic, p-toluenesulfonic, cyclohexanesulfamic, saccharinic or trifluoroacetic acid.

Pharmaceutically acceptable basic/cationic salts include, and are not limited to aluminum, 2-amino-2-hydroxymethyl-propane-1,3-diol (also known as tris(hydroxymethyl)aminomethane, tromethane or “TRIS”), ammonia, benzathine, t-butylamine, calcium, calcium gluconate, calcium hydroxide, chloroprocaine, choline, choline bicarbonate, choline chloride, cyclohexylamine, diethanolamine, ethylenediamine, lithium, LiOMe, L-lysine, magnesium, meglumine, NH₃, NH₄OH, N-methyl-D-glucamine, piperidine, potassium, potassium-t-butoxide, potassium hydroxide (aqueous), procaine, quinine, sodium, sodium carbonate, sodium-2-ethylhexanoate (SEH), sodium hydroxide, triethanolamine or zinc.

### Methods of Use

The present invention is directed to a method for preventing, treating or ameliorating a MMP9 and/or MMP13 mediated syndrome, disorder or disease comprising administering to a subject in need thereof an effective amount of a compound listed in the examples section of this specification or a form, composition or medicament thereof.

Examples of a MMP9 and/or MMP13 mediated syndrome, disorder or disease for which the compounds listed in the examples section of this specification are useful include angiogenesis, osteoarthritis, rheumatoid arthritis, gastric ulcers, pulmonary hypertension, chronic obstructive pulmonary disorder, inflammatory bowel syndrome, periodontal disease, skin ulcers, liver fibrosis, emphysema, Marfan syndrome, stroke, multiple sclerosis, abdominal aortic aneurysm, coronary artery disease, idiopathic pulmonary fibrosis, renal fibrosis, migraine, and cardiovascular disorders including: atherosclerotic plaque, ruptive aneurysm, vascular tissue morphogenesis, and myocardial tissue morphogenesis.

The term “administering” with respect to the methods of the invention, means a method for therapeutically or prophylactically preventing, treating or ameliorating a syndrome, disorder or disease as described herein by using a compound listed in the examples section of this specification or a form, composition or medicament thereof. Such methods include administering an effective amount of said compound, compound form, composition or medicament at different times during the course of a therapy or concurrently in a combination form. The methods of the invention are to be understood as embracing all known therapeutic treatment regimens.

The term “subject” refers to a patient, which may be animal, typically a mammal, typically a human, which has been the object of treatment, observation or experiment. In one aspect of the invention, the subject is at risk of (or susceptible to) developing a syndrome, disorder or disease that is associated with elevated MMP9 and/or MMP13 expression or MMP9 and/or MMP13 overexpression, or a patient with an inflammatory condition that accompanies syndromes, disorders or diseases associated with elevated MMP9 and/or MMP13 expression or MMP9 and/or MMP13 overexpression.

The term “therapeutically effective amount” means that amount of active compound or pharmaceutical agent that elicits the biological or medicinal response in a tissue system, animal or human, that is being sought by a researcher, veterinarian, medical doctor, or other clinician, which includes preventing, treating or ameliorating the symptoms of a syndrome, disorder or disease being treated.

When employed as inhibitors of pro-matrix metalloproteinase activation, the compounds of the invention may be administered in an effective amount within the dosage range of about 0.5 mg to about 10 g, preferably between about 0.5 mg to about 5 g, in single or divided daily doses. The dosage administered will be affected by factors such as the route of administration, the health, weight and age of the recipient, the frequency of the treatment and the presence of concurrent and unrelated treatments.

It is also apparent to one skilled in the art that the therapeutically effective dose for compounds of the present invention or a pharmaceutical composition thereof will vary according to the desired effect. Therefore, optimal dosages to be administered may be readily determined by one skilled in the art and will vary with the particular compound used, the mode of administration, the strength of the preparation, and the advancement of the disease condition. In addition, factors associated with the particular subject being treated, including subject age, weight, diet and time of administration, will result in the need to adjust the dose to an appropriate therapeutic level. The above dosages are thus exemplary of the average case. There can, of course, be individual instances where higher or lower dosage ranges are merited, and such are within the scope of this invention.

The compounds listed in the examples section of this specification may be formulated into pharmaceutical compositions comprising any known pharmaceutically acceptable carriers. Exemplary carriers include, but are not limited to, any suitable solvents, dispersion media, coatings, antibacterial and antifungal agents and isotonic agents. Exemplary excipients that may also be components of the formulation include fillers, binders, disintegrating agents and lubricants.

The pharmaceutically-acceptable salts of the compounds listed in the examples section of this specification include the conventional non-toxic salts or the quaternary ammonium salts which are formed from inorganic or organic acids or bases. Examples of such acid addition salts include acetate, adipate, benzoate, benzenesulfonate, citrate, camphorate, dodecylsulfate, hydrochloride, hydrobromide, lactate, maleate, methanesulfonate, nitrate, oxalate, pivalate, propionate, succinate, sulfate and tartrate. Base salts include ammonium salts, alkali metal salts such as sodium and potassium salts, alkaline earth metal salts such as calcium and magnesium salts, salts with organic bases such as dicyclohexylamino salts and salts with amino acids such as arginine. Also, the basic nitrogen-containing groups may be quaternized with, for example, alkyl halides.

The pharmaceutical compositions of the invention may be administered by any means that accomplish their intended purpose. Examples include administration by parenteral, subcutaneous, intravenous, intramuscular, intraperitoneal, transdermal, buccal or ocular routes. Alternatively or concurrently, administration may be by the oral route. Suitable formulations for parenteral administration include aqueous solutions of the active compounds in water-soluble form, for example, water-soluble salts, acidic solutions, alkaline solutions, dextrose-water solutions, isotonic carbohydrate solutions and cyclodextrin inclusion complexes.

The present invention also encompasses a method of making a pharmaceutical composition comprising mixing a pharmaceutically acceptable carrier with any of the compounds of the present invention. Additionally, the present invention includes pharmaceutical compositions made by mixing a pharmaceutically acceptable carrier with any of the compounds of the present invention. As used herein, the term “composition” is intended to encompass a product comprising the specified ingredients in the specified amounts, as well as any product which results, directly or indirectly, from combinations of the specified ingredients in the specified amounts.

### Polymorphs and Solvates

Furthermore, the compounds of the present invention may have one or more polymorph or amorphous crystalline forms and as such are intended to be included in the scope of the invention. In addition, the compounds may form solvates, for example with water (i.e., hydrates) or common organic solvents. As used herein, the term “solvate” means a physical association of the compounds of the present invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. The term “solvate” is intended to encompass both solution-phase and isolatable solvates. Non-limiting examples of suitable solvates include ethanolates, methanolates, and the like.

It is intended that the present invention include within its scope polymorphs and solvates of the compounds of the present invention. Thus, in the methods of treatment of the present invention, the term “administering” shall encompass the means for treating, ameliorating or preventing a syndrome, disorder or disease described herein with the compounds of the present invention or a polymorph or solvate thereof, which would obviously be included within the scope of the invention albeit not specifically disclosed.

The present invention includes within its scope prodrugs of the compounds of this invention. In general, such prodrugs will be functional derivatives of the compounds which are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the patient.

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention.

Where the processes for the preparation of the compounds according to the invention give rise to mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-D-tartaric acid and/or (+)-di-p-toluoyl-L-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

### EXAMPLES

### Intermediate 1

### 1-(2-Amino-thiazol-5-yl)-2-bromo-ethanone

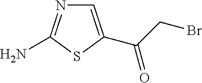

Bromine (0.115 mL, 2.24 mmol) in dioxane (10 mL) was added to a solution of 1-(2-amino-thiazol-5-yl)-ethanone (500 mg, 2.24 mmol, prepared as described in J. Org. Chem. 1984, 49, 566) in 48% aq. HBr (10 mL) at 60° C. The resulting orange solution was stirred at this temperature for 1 h, then was added dropwise to stirred ice-cold sat. aq. NaHCO₃. The mixture was extracted with EtOAc. The organic phase was dried (Na₂SO₄), filtered, and concentrated. The residue was purified by flash column chromatography (silica gel, 20-60% EtOAc-Hept), affording the title compound as a white solid.

### Intermediate 2: step a

### Toluene-4-sulfonic acid 2-oxo-1-propionyl-butyl ester

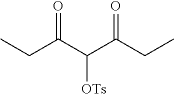

[Hydroxy(tosyloxy)iodo]benzene (4.31 g, 11.0 mmol) was added to a solution of heptane-3,5-dione (1.35 mL, 10.0 mmol, Kodak) in acetonitrile (20 mL). The mixture was heated at reflux for 1 h, then was concentrated and the residue was purified by flash column chromatography (silica gel, 5% EtOAc-Hept), affording the title compound as a faintly brown oil.

### Intermediate 2: step b

### 1-(2-Amino-4-ethyl-thiazol-5-yl)-propan-1-one

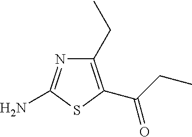

Thiourea (566 mg, 7.44 mmol) was added to a solution of toluene-4-sulfonic acid 2-oxo-1-propionyl-butyl ester (2.22 g, 7.44 mmol, intermediate 2, step a) in acetonitrile (14 mL) and the resulting mixture was heated at reflux for 2 h. The mixture was slowly added to stirred 5% w/v aq. NaOH. The mixture was extracted with EtOAc. The organic phase was dried (Na₂SO₄), filtered, and concentrated and the residue was purified by flash column chromatography (silica gel, 30-80% EtOAc-Hept), affording the title compound as a white powder.

### Intermediate 2: step c

### 1-(2-Amino-4-ethyl-thiazol-5-yl)-2-bromo-propan-1-one

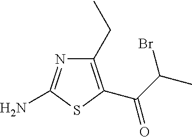

The title compound was prepared using 1-(2-amino-4-ethyl-thiazol-5-yl)-propan-1-one (intermediate 2, step b) in place of 1-(2-amino-thiazol-5-yl)-ethanone according to the procedure described for intermediate 1.

### Intermediate 3

### 2-Bromo-1-(2,4-dimethyl-thiazol-5-yl)-ethanone.HBr

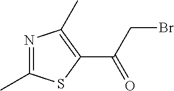

A suspension of bromine (11.9 mL, 231.5 mmol) in 1,4-dioxane (200 mL) was added to a stirred solution of 1-(2,4-dimethyl-thiazol-5-yl)-ethanone (28.75 g, 185.2 mmol, Alfa) in 1,4-dioxane (200 mL). The mixture was stirred for 25 h at 50° C. and the resulting cream-colored suspension was allowed to cool to room temperature and was filtered and washed with 2:1 heptane:EtOAc (v/v). The resulting white powder was recrystallized from EtOH, affording the title compound.

### Intermediate 4

### 1-(2-Amino-4-methyl-thiazol-5-yl)-2-bromo-ethanone

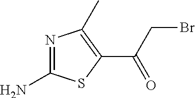

1-(2-Amino-4-methyl-thiazol-5-yl)-2-bromo-ethanone.HBr was prepared as described in WO 2005/068444. To convert to the corresponding free base, the crude reaction mixture was slowly added to an ice-cold sat. aq. NaHCO₃ solution. The precipitate was collected by vacuum filtration and washed with Et₂O. The crude product was recrystallized from EtOH, affording the title compound as an orange powder.

### Intermediate 5

### N-[5-(2-Bromo-acetyl)-4-methyl-thiazol-2-yl]-acetamide.HBr

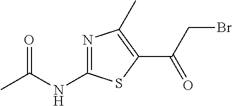

The title compound was prepared as described in WO 2005/068444.

### Intermediate 6: step a

### Toluene-4-sulfonic acid 1-acetyl-3,3,3-trifluoro-2-oxo-propyl ester

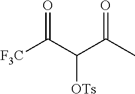

A solution of 1,1,1-trifluoro-2,4-pentanedione (3.00 g, 19.47 mmol) and [hydroxyl(tosyloxy)iodo]benzene (9.16 g, 23.36 mmol) in acetonitrile (100 mL) was heated to 45° C. for 45 minutes. The reaction mixture was then cooled to room temperature, evaporated and purified via column chromatography eluting with heptanes: ethyl acetate to give the title compound.

### Intermediate 6: step b

### 1-(2-Amino-4-trifluoromethyl-thiazol-5-yl)-ethanone

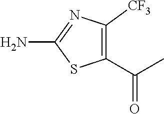

A solution of toluene-4-sulfonic acid 1-acetyl-3,3,3-trifluoro-2-oxo-propyl ester (0.100 g, 0.308 mmol, intermediate 6, step a) and thiourea (0.028 g, 0.370 mmol) in acetonitrile (5 mL) were heated to reflux for several hours. The reaction mixture was then cooled to room temperature and evaporated. Ethyl acetate was added and the solution was filtered. The filtrate was evaporated, dichloromethane was added and the solution was filtered to give the title compound as a solid.

### Intermediate 6: step c

### 1-(2-Amino-4-trifluoromethyl-thiazol-5-yl)-2-bromo-ethanone

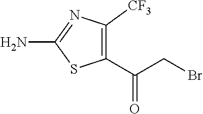

A solution of bromine (0.024 mL, 0.475 mmol) in dioxane (3 mL) was added dropwise to a solution of 1-(2-amino-4-trifluoromethyl-thiazol-5-yl)-ethanone (0.100 g, 0.475 mmol, intermediate 6, step b) in 48% aqueous HBr (3 mL) at 60° C. and stirred for 2 hours. The reaction mixture was cooled to room temperature, saturated aqueous NaHCO₃ was added slowly and the pH was adjusted to 7 with 2 N aqueous Na₂CO₃. Ethyl acetate was added and the product was extracted, dried with sodium sulfate and evaporated to give the title compound.

### Intermediate 7: step a

### 1-(4-Methyl-2-methylamino-thiazol-5-yl)-ethanone.HCl

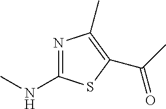

### 3-chloro-pentane-2,4-dione (6.46 g, 48 mmol) and pyridine (3 mL) were added sequentially to a solution of methylthiourea (5 g, 48 mmol) in MeOH (50 mL). A white solid precipitated after 10 min. The mixture was stirred at room temperature for 12 h and the precipitate was collected by vacuum filtration and washed with diisopropyl ether to afford the title compound.

### Intermediate 7: step b

### 2-Bromo-1-(4-methyl-2-methylamino-thiazol-5-yl)-ethanone.HBr

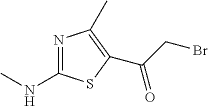

Pyridinium tribromide (9 g, 28 mmol) was added to a solution of 1-(4-methyl-2-methylamino-thiazol-5-yl)-ethanone.HCl (3 g, 14.5 mmol, intermediate 7, step a) in 30% aq. acetic acid (30 mL). The resulting mixture was stirred at room temperature for 2 h. The precipitate was collected by vacuum filtration and washed with diisopropyl ether to yield the title compound.

### Intermediate 8: step a

### 1-Isopropoxy-2-isothiocyanato-benzene

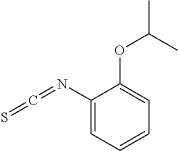

2-Isopropoxy-phenylamine.HCl (Chembridge, 1.0 g, 5.33 mmol) was partitioned between EtOAc and sat. aq. NaHCO₃ to convert to the free base. The organic phase was dried (Na₂SO₄), filtered, and concentrated, yielding the corresponding free base as a light pink liquid.

A solution of sodium bicarbonate (1.34 g, 16.0 mmol) in water (30 mL) was added to 2-isopropoxy-phenylamine (crude free base prepared above) in a mixture of chloroform (25 mL) and water (25 mL). Thiophosgene (0.429 mL, 5.60 mmol) was then added. The biphasic solution was stirred at room temperature overnight. TLC analysis indicated slight remaining starting material, so an additional 0.061 mL portion of thiophosgene was added and the mixture was stirred for 20 min. The phases were separated and the aqueous phase was extracted with CH₂Cl₂. The organic phase was dried (Na₂SO₄), filtered, and concentrated, yielding the crude title compound as a light brown oil.

### Intermediate 8: step b

### (2-Isopropoxy-phenyl)-thiourea

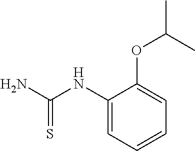

Crude 1-isopropoxy-2-isothiocyanato-benzene (1.03 g, 5.33 mmol, intermediate 8, step a) was treated with 2 M ammonia in MeOH (20 mL) and the resulting solution was stirred at 23° C. for 3 h. The mixture was concentrated and the residue was purified by column chromatography (silica gel, 30-50% EtOAc-Hept), affording the title compound.

### Intermediate 9: step a

### 4-Fluoro-2-isothiocyanato-1-methoxy-benzene

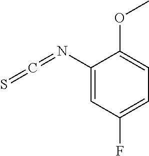

The title compound was prepared using 5-fluoro-2-methoxy-phenylamine (Aldrich) in place of 2-isopropoxy-phenylamine according to the procedure of intermediate 8, step a.

### Intermediate 9: step b

### (5-Fluoro-2-methoxy-phenyl)-thiourea

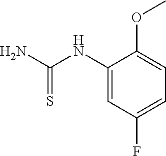

The title compound was prepared using crude 4-fluoro-2-isothiocyanato-1-methoxy-benzene (intermediate 9, step a) in place of 1-isopropoxy-2-isothiocyanato-benzene according to the procedure of intermediate 8, step b (reaction time 16 h) and was purified by flash column chromatography (silica gel, 0-3% MeOH—CH₂Cl₂) and triturated with heptane.

### Intermediate 10

### (3-Chloro-2-methoxy-phenyl)-thiourea

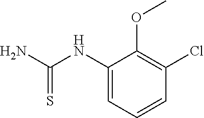

To a solution of 3-chloro-2-methoxy-phenylamine (2.36 g, 15.0 mmol, Aldrich) in acetone (30 mL) at reflux was slowly added benzoyl isothiocyanate (2.22 mL, 16.5 mmol) and the mixture was stirred at reflux for 30 min, then was poured into a mixture of ice and water. The precipitate was collected by vacuum filtration and was treated with 10% aq. NaOH (15 mL). The mixture was heated to reflux for 40 min, and was cooled to room temperature. A white solid precipitated and was collected by vacuum filtration, affording the crude title compound which was used without further purification.

### Intermediate 11: step a

### 3-Isothiocyanato-benzamide

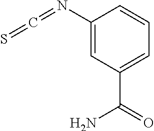

A solution of sodium bicarbonate (3.78 g, 45.0 mmol) in water (80 mL) was added to 3-amino-benzamide (2.04 g, 15.0 mmol, TCI) in a mixture of chloroform (75 mL) and water (75 mL). Thiophosgene (1.21 mL, 15.75 mmol) was then added. The biphasic solution was stirred at room temperature for 1 h. The phases were separated. The aqueous phase contained a white precipitate, which was collected by vacuum filtration, affording the crude title compound.

### Intermediate 11: step b

### 3-Thioureido-benzamide

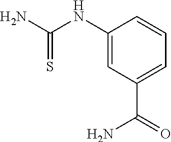

Crude 3-isothiocyanato-benzamide (2.3 g, 12.9 mmol, intermediate 11, step a) was suspended in MeOH (10 mL) and treated with 2 N NH₃ in MeOH (30 mL). The mixture was stirred overnight at room temperature and the white precipitate was collected by vacuum filtration, affording the title compound.

### Intermediate 12: step a

### 4-Fluoro-1-isopropoxy-2-isothiocyanato-benzene

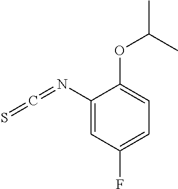

The title compound was prepared using 5-fluoro-2-isopropoxy-phenylamine (Combi-Blocks) in place of 2-isopropoxy-phenylamine according to the procedure described for intermediate 8, step a (reaction time 3 h).

### Intermediate 12: step b

### (5-Fluoro-2-isopropoxy-phenyl)-thiourea

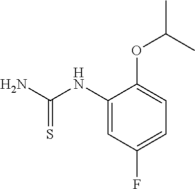

The title compound was prepared using 4-fluoro-1-isopropoxy-2-isothiocyanato-benzene (intermediate 12, step a) in place of 1-isopropoxy-2-isothiocyanato-benzene according to the procedure described for intermediate 8, step b (reaction temperature 40° C., reaction time 30 min), except that the crude product obtained from concentration of the reaction mixture was used in the next reactions.

### Intermediate 13: step a

### 1-Isothiocyanato-2-trifluoromethoxy-benzene

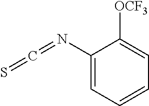

The title compound was prepared using commercially available 2-(trifluoromethyl)-aniline in place of 2-isopropoxy-phenylamine according to the procedure described for intermediate 8, step a (reaction time 3 h).

### Intermediate 13: step b

### (2-Trifluoromethoxy-phenyl)-thiourea

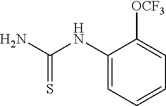

The title compound was prepared using 1-isothiocyanato-2-trifluoromethoxy-benzene (intermediate 13, step a) in place of in place of 1-isopropoxy-2-isothiocyanato-benzene according to the procedure described for intermediate 8, step b (reaction temperature 40° C., reaction time 1 h), except that the crude product obtained from concentration of the reaction mixture was used in the next reactions.

### Intermediate 14: step a

### 4-Bromo-2-isothiocyanato-1-methoxy-benzene

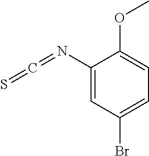

A solution of sodium bicarbonate (3.74 g, 44.5 mmol) in water (75 mL) was added to commercially available 5-bromo-2-methoxy-phenylamine (3 g, 14.8 mmol) in chloroform (75 mL). Thiophosgene (1.42 mL, 18.6 mmol) was then added. The biphasic solution was stirred at room temperature for 1 h. The phases were separated and the aqueous phase was extracted with CH₂Cl₂. The organic phase was dried (Na₂SO₄), filtered, and concentrated, yielding the crude title compound as an off-white solid.

### Intermediate 14: step b

### (5-Bromo-2-methoxy-phenyl)-thiourea

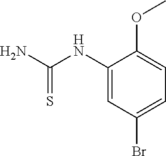

Crude 4-bromo-2-isothiocyanato-1-methoxy-benzene (3.6 g, 14.7 mmol, intermediate 14, step a) was suspended in MeOH (10 mL). A 2 M solution of ammonia in MeOH (56.5 mL) was added and the resulting yellow solution was stirred at room temperature for 16 h. The reaction mixture was concentrated to afford the title compound as a white powder.

### Intermediate 15

### N-[2-(2-Hydroxy-phenylamino)-4′-methyl-[4,5′]bithiazolyl-2′-yl]-acetamide.HBr

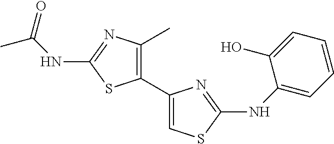

A solution of N-[5-(2-bromo-acetyl)-4-methyl-thiazol-2-yl]-acetamide.HBr (2.00 g, 5.59 mmol, intermediate 5) and commercially available (2-hydroxy-phenyl)-thiourea (0.940 g, 5.59 mmol) in ethanol (25 mL) was stirred at room temperature for several hours. The reaction mixture was then filtered and washed with ethanol and dried to give the title compound.

### Intermediate 16

### 2-(4-Pyridin-3-yl-thiazol-2-ylamino)-phenol

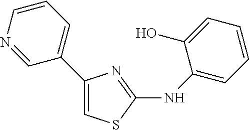

A solution of 2-bromo-1-pyridin-3-yl-ethanone.HBr (2.00 g, 7.12 mmol), commercially available (2-hydroxy-phenyl)-thiourea (1.19 g, 7.12 mmol), and triethylamine (1.98 mL, 14.24 mmol) in THF (20 mL) was heated to 80° C. for 5 hours. The reaction mixture was then cooled to room temperature, evaporated and purified via column chromatography with heptanes: ethyl acetate to give the title compound.

### Intermediate 17: step a

### 1-Methyl-1H-benzoimidazole-5-carboxylic acid methoxy-methyl-amide

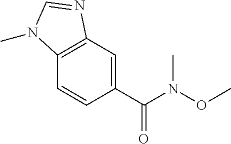

To a mixture of 1-methyl-1H-benzoimidazole-5-carbonyl chloride.HCl (1 g, 4.33 mmol) and O,N-dimethyl-hydroxylamine.HCl (422 mg, 4.33 mmol) in dichloromethane (15 mL) was added Et₃N (2.4 mL, 17.3 mmol) dropwise. The reaction mixture was stirred for 24 h. Water (20 mL) was added and the solution was extracted with dichloromethane. The organic extracts were dried (Na₂SO₄), filtered, concentrated and purified through column chromatography to afford the title compound as a white solid.

### Intermediate 17: step b

### 1-(1-Methyl-1H-benzoimidazol-5-yl)-ethanone

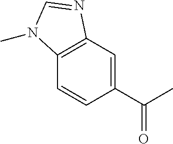

To a mixture of 1-methyl-1H-benzoimidazole-5-carboxylic acid methoxy-methyl-amide (700 mg, 3.19 mmol, intermediate 17, step a) in THF (20 mL) at 0° C. was added MeMgBr (3 M in ether, 2.18 mL, 6.55 mmol) dropwise. The reaction mixture was warmed up to room temperature and stirred for 2 h. Saturated aq NH₄Cl (2 mL) was added to quench the reaction and the solution was extracted with EtOAc. The organic extracts were dried (Na₂SO₄), filtered, concentrated and purified through column chromatography to afford the title compound as a white solid.

### Intermediate 17: step c

### 2-Bromo-1-(1-methyl-1H-benzoimidazol-5-yl)-ethanone.HBr

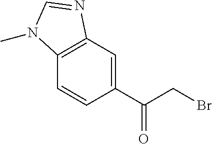

To 1-(1-methyl-1H-benzoimidazol-5-yl)-ethanone (600 mg, 3.44 mmol, intermediate 17, step b) in 48% HBr (5 mL) at 60° C. was added 0.778 M Br₂ in 1,4-dioxane (4.43 mL, 3.44 mmol). The mixture was stirred for 18 h at 60° C., concentrated and dried under vacuum to give the title compound.

### Intermediate 18: step a

### 1-(2,4-Dimethyl-imidazol-1-yl)-ethanone

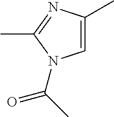

The title compound was prepared according to the procedure described in J. Org. Chem. 1983, 48, 897: To a solution of 2,4-dimethylimidazole (4.00 g, 41.6 mmol) in toluene/chloroform (1/1, v/v, 50 mL) at room temperature was added acetyl chloride (1.48 mL, 20.8 mmol) dropwise over several minutes. The reaction was stirred at room temperature for 2 hours and filtered. The filtrate was evaporated, ethyl acetate was added and the solution was filtered again. The filtrate was evaporated to give the title compound.

### Intermediate 18: step b

### 1-(2,5-Dimethyl-3H-imidazol-4-yl)-ethanone

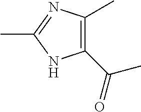

The title compound was prepared according to the procedure described in J. Org. Chem. 1983, 48, 897. A solution of 1-(2,4-dimethyl-imidazol-1-yl)-ethanone (2.00 g, 14.5 mmol, intermediate 18, step a) in THF (75 mL) was added to quartz test tubes and placed in a Rayonet UV light box for 18 hours. The reaction was then evaporated and purified via column chromatography with 5% methanol in dichloromethane to give the title compound.

### Intermediate 18: step c

### 1-(1-Acetyl-2,5-dimethyl-1H-imidazol-4-yl)-ethanone

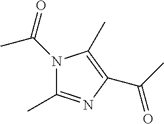

According to the general method described in J. Org. Chem. 1987, 52, 2714, acetyl chloride (0.199 mL, 2.79 mmol) was added to a mixture of 1-(2,5-dimethyl-3H-imidazol-4-yl)-ethanone (350.9 mg, 2.54 mmol, intermediate 18, step b) and triethylamine (0.388 mL, 2.79 mmol) in chloroform (15 mL). The resulting yellow solution was stirred at room temperature for 21 h. The mixture was diluted with CH₂Cl₂ and was washed three times with water. The organic phase was dried (Na₂SO₄), filtered, and concentrated, yielding the crude title compound as a light yellow oil.

### Intermediate 18: step d

### 1-(2,3,5-Trimethyl-3H-imidazol-4-yl)-ethanone

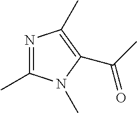

According to the general method described in J. Org. Chem. 1987, 52, 2714, trimethyloxonium tetrafluoroborate (563.5 mg, 3.81 mmol) was added to a solution of 1-(1-acetyl-2,5-dimethyl-1H-imidazol-4-yl)-ethanone (457.7 mg, 2.54 mmol, intermediate 18, step c) in CH₂Cl₂ (10 mL). The reaction mixture was stirred at room temperature for 24 h. The mixture was concentrated and the residue was treated with water (10 mL) and was basified by addition of solid Na₂CO₃. The mixture was extracted five times with chloroform. The organic phase was dried (Na₂SO₄), filtered, and concentrated, yielding a light yellow oil which was purified by flash column chromatography (silica gel, 0-10% MeOH—CH₂Cl₂), affording the title compound as a white solid (¹H NMR integration indicated a 19:1 mixture of title compound: regioisomeric byproduct 1-(1,2,5-trimethyl-1H-imidazol-4-yl)-ethanone).

### Intermediate 18: step e

### 2-Bromo-1-(2,3,5-trimethyl-3H-imidazol-4-yl)-ethanone

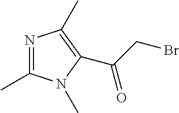

Bromine (0.0464 mL, 0.903 mmol) was added to a solution of 1-(2,3,5-trimethyl-3H-imidazol-4-yl)-ethanone (125 mg, 0.821 mmol, intermediate 18, step d) in 48% aq. HBr (2 mL) and the resulting mixture was stirred in a 60° C. oil bath for 1.5 h. The reaction mixture was diluted with 10 mL water and was slowly added to sat. aq. NaHCO₃ (final pH 8). The mixture was extracted with CH₂Cl₂ and the organic phase was dried (Na₂SO₄), filtered, and concentrated. The residue was purified by flash column chromatography (silica gel, 0-1% MeOH—CH₂Cl₂), affording the title compound as a white crystalline solid.

### Intermediate 19: step a

### 1-(2,4-Dimethylthiazol-5-yl)propan-1-one

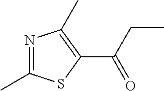

Lithium hexamethyldisilazide (1 M in THF, 21.3 mL, 21.3 mmol) was added to a solution of 1-(2,4-dimethylthiazol-5-yl)ethanone (Alfa, 3.0 g, 19.3 mmol) in THF (20 mL) at −78° C. The resulting yellow solution was stirred at −78° C. for 30 min before addition of iodomethane (1.33 mL, 21.3 mmol). The resulting yellow solution was stirred at −78° C. for 30 min, then at 0° C. for 30 min. Saturated aq. NH₄Cl was added and the mixture was partially concentrated to remove THF. The aqueous residue was extracted with EtOAc and the organic extracts were dried (Na₂SO₄), filtered, and concentrated. The crude product was purified by column chromatography (Silica gel, 0-30% EtOAc-hept), affording the title compound as a light yellow liquid.

### Intermediate 19: step b

### 2-Bromo-1-(2,4-dimethylthiazol-5-yl)propan-1-one

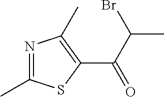

To 1-(2,4-dimethylthiazol-5-yl)propan-1-one (631 mg, 3.73 mmol, intermediate 19, step a) in 1,4-dioxane (6 mL) was added bromine (0.191 mL, 3.73 mmol) in 1,4-dioxane (6 mL). The resulting orange solution was heated in a 50° C. oil bath for 3 d. The reaction mixture was filtered and the collected solid was partitioned between EtOAc and sat. aq. NaHCO₃. The separated aq. phase was extracted with EtOAc and the organic extracts were dried (Na₂SO₄), filtered, and concentrated. The residue was purified by column chromatography (Silica gel, 1-5% Et₂O-DCM), affording the title compound as a colorless liquid.

### Intermediate 20: step a

### 1-Methylpiperidine-4-carbothioamide

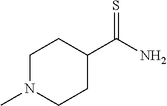

To a suspension of 1-methylpiperidine-4-carboxamide (3.97 g, 27.9 mmol, Amfinecom) in a mixture of toluene (70 mL) and THF (30 mL) was added Lawesson's reagent (6.78 g, 16.8 mmol). The resulting light yellow suspension was heated at reflux for 22 h. The reaction mixture was diluted with DCM and MeOH and was concentrated onto silica gel for purification by column chromatography (Silica gel, 1-8% MeOH in 98:2 DCM:conc. aq. NH₄OH, water layer removed in a separatory funnel), affording the title compound as a yellow solid.

### Intermediate 20: step b

### 1-(4-Methyl-2-(1-methylpiperidin-4-yl)thiazol-5-yl)ethanone

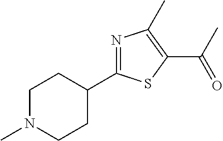

To a suspension of 1-methylpiperidine-4-carbothioamide (1.80 g, 11.4 mmol, intermediate 20, step a) in EtOH (40 mL) was added 3-chloropentane-2,4-dione (1.55 mL, 13.6 mmol) and the mixture was heated at reflux overnight. The reaction mixture was partitioned between EtOAc and sat. aq. NaHCO₃. The aq. phase was extracted with EtOAc. The organic extracts were dried (Na₂SO₄), filtered, and concentrated. The residue was purified by column chromatography (Silica gel, 1-5% MeOH in 98:2 DCM:conc. aq. NH₄OH, water layer removed in a separatory funnel), affording the title compound.

### Intermediate 20: step c

### 2-bromo-1-(4-methyl-2-(1-methylpiperidin-4-yl)thiazol-5-yl)ethanone.HBr

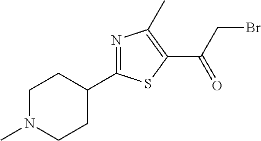

A solution of bromine (0.064 mL, 1.25 mmol) in 48% aq. HBr (0.5 mL) was added to a mixture of 1-(4-methyl-2-(1-methylpiperidin-4-yl)thiazol-5-yl)ethanone (270 mg, 1.13 mmol, intermediate 20, step b) in 48% aq. HBr (2.0 mL). The reaction mixture was heated in a 60° C. oil bath for 75 min. The mixture was concentrated from toluene three times, affording the crude title compound as a thick brown oil which was used without further purification in the next step.

### Intermediate 21: step a

### 1-(6-(4-Cyclopropylpiperazin-1-yl)pyridin-3-yl)ethanone

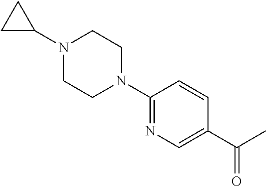

A solution of 1-cyclopropylpiperazine (0.162 g, 1.29 mmol), 1-(6-chloropyridin-3-yl)ethanone

(0.200 g, 1.29 mmol) and DMSO (0.2 mL) was heated to 100° C. overnight. The reaction was then cooled to room temperature, ethyl acetate was added and the reaction mixture was filtered to give the title compound as a solid.

### Intermediate 21: step b

### 2-Bromo-1-(6-(4-cyclopropylpiperazin-1-yl)pyridin-3-yl)ethanone.HBr

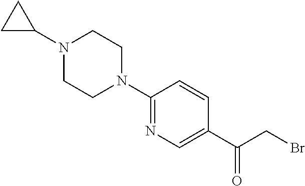

Bromine (0.020 mL, 0.393 mmol) was added to a solution of 1-(6-(4-cyclopropylpiperazin-1-yl)pyridin-3-yl)ethanone (0.107 g, 0.436 mmol) in 48% aqueous HBr (6 mL) at 70° C. and heated at that temperature overnight. The reaction was then cooled to room temperature and was evaporated several times in the presence of toluene to give the title compound.

### Example 1

### N-[2-(2-Isopropoxy-phenylamino)-4′-methyl-[4,5′]bithiazolyl-2′-yl]-acetamide

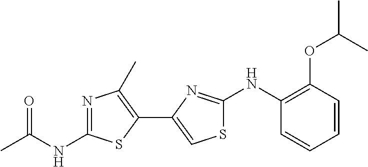

A mixture of N-[5-(2-bromo-acetyl)-4-methyl-thiazol-2-yl]-acetamide.HBr (170.3 mg, 0.476 mmol, intermediate 5), (2-isopropoxy-phenyl)-thiourea (100 mg, 0.476 mmol, intermediate 8, step b), and EtOH (2 mL) was stirred at room temperature. The reaction mixture became a solution, then precipitated a large volume of solid. After 1.5 h, the solid was collected by vacuum filtration and was washed with EtOH. The solid was vigorously stirred in a mixture of sat. aq. NaHCO₃ and EtOAc until it dissolved (10 min). The organic phase was dried (Na₂SO₄), filtered, and concentrated, yielding the title compound as an off-white powder. ¹H NMR (300 MHz, DMSO-d₆) δ ppm 12.05 (s, 1H), 9.34 (s, 1H), 8.33 (dd, J=7.2, 2.3 Hz, 1H), 7.03-7.08 (m, 1H), 6.86-7.00 (m, 3H), 4.66 (sept, J=6.0 Hz, 1H), 2.48 (s, 3H), 2.14 (s, 3H), 1.32 (d, J=6.0 Hz, 6H). MS m/e 389.1 (M+H).

### Example 2

### (2′,4′-Dimethyl-[4,5′]bithiazolyl-2-yl)-(2-isopropoxy-phenyl)-amine

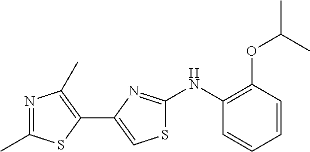

The title compound was prepared using 2-bromo-1-(2,4-dimethyl-thiazol-5-yl)-ethanone.HBr (intermediate 3) in place of N-[5-(2-bromo-acetyl)-4-methyl-thiazol-2-yl]-acetamide.HBr as described in example 1 (reaction time 19 h). ¹H NMR (300 MHz, DMSO-d₆) δ ppm 9.37 (s, 1H), 8.34 (dd, J=7.5, 2.3 Hz, 1H), 7.02-7.07 (m, 1H), 6.88-6.99 (m, 3H), 4.65 (sept, J=6.0 Hz, 1H), 2.59 (s, 3H), 2.51 (s, 3H), 1.32 (d, J=6.0 Hz, 6H). MS m/e 346.0 (M+H).

### Example 3

### N²-(2-Isopropoxy-phenyl)-4′-methyl-[4,5′]bithiazolyl-2,2′-diamine.TFA

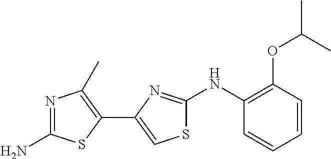

The title compound was prepared using 1-(2-amino-4-methyl-thiazol-5-yl)-2-bromo-ethanone

(intermediate 4) in place of N-[5-(2-bromo-acetyl)-4-methyl-thiazol-2-yl]-acetamide.HBr as described in example 1 (reaction time 20 h). Following preparation of the free base as in example 1, the compound was purified by RP-HPLC (10-90% CH₃CN—H₂O, 0.1% TFA). ¹H NMR (300 MHz, DMSO-d₆) δ ppm 9.43 (s, 1H), 8.98 (br. s., 2H), 8.22 (dd, J=7.9, 1.5 Hz, 1H), 6.99-7.04 (m, 1H), 6.82-6.97 (m, 3H), 4.61 (sept, J=6.0 Hz, 1H), 2.35 (s, 3H), 1.27 (d, J=6.0 Hz, 6H). MS m/e 347.1.

### Example 4

### N²-(5-Fluoro-2-methoxy-phenyl)-4′-methyl-[4,5′]bithiazolyl-2,2′-diamine.HBr

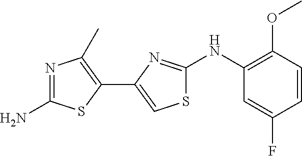

A mixture of 1-(2-amino-4-methyl-thiazol-5-yl)-2-bromo-ethanone (47 mg, 0.20 mmol, intermediate 4) and (5-fluoro-2-methoxy-phenyl)-thiourea (40 mg, 0.20 mmol, intermediate 9, step b) in EtOH (1 mL) was stirred at room temperature for 3 d. The mixture was filtered, washed sequentially with EtOH and heptanes, and air-dried, affording the title compound. ¹H NMR (300 MHz, DMSO-d₆) δ ppm 10.08 (s, 1H), 9.16 (br. s., 2H), 8.33 (dd, J=11.7, 3.0 Hz, 1 H), 7.10 (s, 1H), 7.03 (dd, J=9.2, 5.5 Hz, 1H), 6.77 (td, J=8.5, 3.0 Hz, 1H), 3.87 (s, 3H), 2.41 (s, 3H). MS m/e 337.0 (M+H).

### Example 5

### (2′,4′-Dimethyl-[4,5′]bithiazolyl-2-yl)-(5-fluoro-2-methoxy-phenyl)-amine.HBr

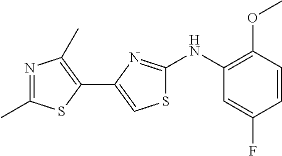

The title compound was prepared using 2-bromo-1-(2,4-dimethyl-thiazol-5-yl)-ethanone.HBr (intermediate 3) in place of 1-(2-amino-4-methyl-thiazol-5-yl)-2-bromo-ethanone as described in example 4. The crude product was purified by recrystallization from EtOH, affording the title compound as a light yellow powder. ¹H NMR (300 MHz, DMSO-d₆) δ ppm 10.02 (s, 1H), 8.42 (dd, J=11.7, 3.0 Hz, 1H), 7.12 (s, 1H), 7.02 (dd, J=8.9, 5.5 Hz, 1H), 6.76 (td, J=8.6, 3.1 Hz, 1 H), 3.87 (s, 3H), 2.69 (s, 3H), 2.55 (s, 3H). MS m/e 336.1 (M+H).

### Example 6

### N-[2-(5-Fluoro-2-methoxy-phenylamino)-4′-methyl-[4,5′]bithiazolyl-2′-yl]-acetamide.HBr)

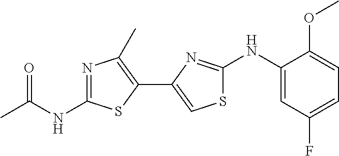

The title compound was prepared using N-[5-(2-bromo-acetyl)-4-methyl-thiazol-2-yl]-acetamide.HBr (intermediate 5) in place of 1-(2-amino-4-methyl-thiazol-5-yl)-2-bromo-ethanone as described in example 4. ¹H NMR (300 MHz, DMSO-d₆) δ 12.08 (br. s., 1H), 9.94 (s, 1H), 8.46 (dd, J=3.20, 11.87 Hz, 1H), 7.01 (dd, J=5.27, 9.04 Hz, 1H), 6.94 (s, 1H), 6.75 (td, J=3.39, 8.48 Hz, 1H), 3.87 (s, 3H), 2.49 (s, 3H), 2.14 (s, 3H). MS m/e 379.0 (M+H).

### Example 7

### N²-(3-Chloro-2-methoxy-phenyl)-4′-methyl-[4,5′]bithiazolyl-2,2′-diamine.HBr

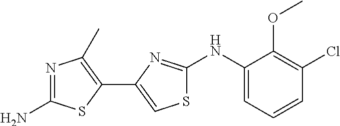

The title compound was prepared using (3-chloro-2-methoxy-phenyl)-thiourea (intermediate 10) in place of (5-fluoro-2-methoxy-phenyl)-thiourea according to the procedure of example 4. ¹H NMR (300 MHz, DMSO-d₆) δ 10.13 (s, 1H), 9.14 (br. s., 2H), 8.40 (dd, J=2.64, 7.16 Hz, 1H), 7.06-7.17 (m, 3H), 3.79 (s, 3H), 2.41 (s, 3H). MS m/e 353.1; 355.1 (M+H).

### Example 8

### 3-(2′-Amino-4′-methyl-[4,5′]bithiazolyl-2-ylamino)-benzamide.HBr

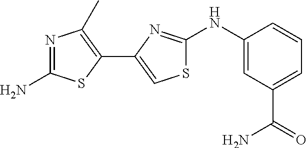

The title compound was prepared using 3-thioureido-benzamide (intermediate 11, step b) in place of (5-fluoro-2-methoxy-phenyl)-thiourea according to the procedure of example 4 (reaction time 1 d). The crude product was recrystallized from a mixture of EtOH and water, affording the title compound. ¹H NMR (300 MHz, DMSO-d₆) δ 10.57 (s, 1H), 9.21 (br. s., 2H), 8.09 (s, 1H), 7.95 (br. s., 1H), 7.79 (d, J=7.91 Hz, 1H), 7.29-7.53 (m, 3H), 7.08 (s, 1H), 2.44 (s, 3H). MS m/e 332.1 (M+H).

### Example 9

### 3-(2′,4′-Dimethyl-[4,5′]bithiazolyl-2-ylamino)-benzamide

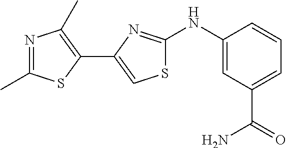

A mixture of 3-thioureido-benzamide (100 mg, 0.512 mmol, intermediate 11, step b), 2-bromo-1-(2,4-dimethyl-thiazol-5-yl)-ethanone.HBr (161.3 mg, 0.512 mmol, intermediate 3), and EtOH (2.0 mL) was stirred at room temperature for 8 d. The mixture was partitioned between sat. aq. NaHCO₃ and EtOAc. The aq. phase was extracted with EtOAc. The organic phase was dried (Na₂SO₄), filtered, and concentrated and the residue was purified by column chromatography (silica gel, 1-10% MeOH—CH₂Cl₂), affording the title compound. ¹H NMR (300 MHz, DMSO-d₆) δ 10.46 (s, 1H), 8.05 (s, 1H), 7.84-7.99 (m, 2H), 7.28-7.50 (m, 3H), 6.99 (s, 1H), 2.60 (s, 3H), 2.54 (s, 3H). MS m/e 331.0 (M+H).

### Example 10

### N²-(2-Methoxy-phenyl)-[4,5′]bithiazolyl-2,2′-diamine.HBr

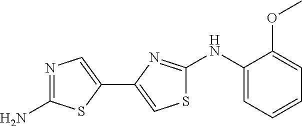

The title compound was prepared using 1-(2-amino-thiazol-5-yl)-2-bromo-ethanone (intermediate 1) and commercially available (2-methoxy-phenyl)-thiourea in place of 1-(2-amino-4-methyl-thiazol-5-yl)-2-bromo-ethanone and (5-fluoro-2-methoxy-phenyl)-thiourea, respectively, according to the procedure of example 4 (reaction time 1 d). ¹H NMR (400 MHz, DMSO-d₆) δ 9.76 (s, 1H), 9.13 (br. s., 2H), 8.30 (dd, J=1.47, 7.82 Hz, 1H), 7.70 (s, 1H), 7.22 (s, 1H), 7.05 (dd, J=1.22, 7.83 Hz, 1H), 7.01 (td, J=1.47, 7.58 Hz, 1H), 6.94 (dt, J=1.47, 7.58 Hz, 1H), 3.86 (s, 3H). MS m/e 305.1 (M+H).

### Example 11

### 4′-Ethyl-N²-(2-methoxy-phenyl)-5-methyl-[4,5′]bithiazolyl-2,2′-diamine

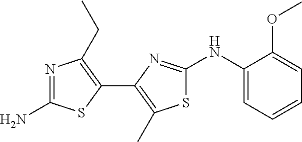

A mixture of 1-(2-amino-4-ethyl-thiazol-5-yl)-2-bromo-propan-1-one (78.9 mg, 0.300 mmol, intermediate 2, step c) and commercially available (2-methoxy-phenyl)-thiourea (54.7 mg, 0.300 mmol) in EtOH (1.2 mL) was heated by microwave irradiation (80° C., 10 min, 300 W). The reaction mixture was partitioned between EtOAc and sat. aq. NaHCO₃. The separated aq. phase was extracted twice with EtOAc. The organic phase was dried (Na₂SO₄), filtered, and concentrated and the residue was purified by column chromatography (silica gel, first column 20-80% EtOAc-Hept; second column 0-2.5% MeOH—CH₂Cl₂) to afford the title compound. ¹H NMR (300 MHz, DMSO-d₆) δ 9.34 (s, 1H), 8.34 (d, J=7.54 Hz, 1H), 6.82-7.05 (m, 5H), 3.85 (s, 3H), 2.44 (q, J=7.35 Hz, 2H), 2.21 (s, 3H), 1.13 (t, J=7.35 Hz, 3H). MS m/e 347.1 (M+H).

### Example 12

### N-[2-(5-Fluoro-2-isopropoxy-phenylamino)-4′-methyl-[4,5′]bithiazolyl-2′-yl]-acetamide

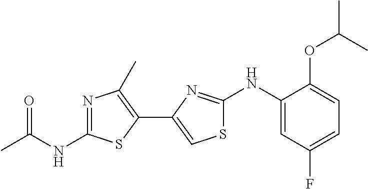

The title compound was prepared using (5-fluoro-2-isopropoxy-phenyl)-thiourea (intermediate 12, step b) in place of (2-isopropoxy-phenyl)-thiourea as described in example 1 (reaction time 2 d). ¹H NMR (400 MHz, DMSO-d₆) δ 12.05 (s, 1H), 9.34 (s, 1H), 8.25 (dd, J=6.36, 9.05 Hz, 1H), 7.01 (dd, J=2.69, 10.76 Hz, 1H), 6.85 (s, 1H), 6.75 (td, J=2.81, 8.62 Hz, 1H), 4.70 (sept, J=5.93 Hz, 1H), 2.46 (s, 3H), 2.13 (s, 3H), 1.32 (d, J=5.87 Hz, 6H). MS m/e 407.0 (M+H).

### Example 13

### (2′,4′-Dimethyl-[4,5′]bithiazolyl-2-yl)-(5-fluoro-2-isopropoxy-phenyl)-amine

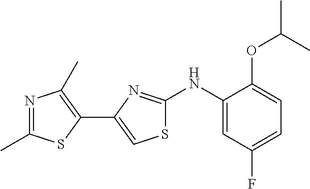

The title compound was prepared using (5-fluoro-2-isopropoxy-phenyl)-thiourea (intermediate 12, step b) in place of (2-isopropoxy-phenyl)-thiourea and 2-bromo-1-(2,4-dimethyl-thiazol-5-yl)-ethanone.HBr (intermediate 3) in place of N-[5-(2-bromo-acetyl)-4-methyl-thiazol-2-yl]-acetamide.HBr as described in example 1 (reaction time 3 d). ¹H NMR (400 MHz, DMSO-d₆) δ 9.38 (s, 1H), 8.28 (dd, J=6.48, 8.93 Hz, 1H), 7.00 (dd, J=2.81, 10.88 Hz, 1H), 6.91 (s, 1H), 6.75 (td, J=2.81, 8.62 Hz, 1H), 4.69 (sept, J=6.11 Hz, 1H), 2.59 (s, 3H), 1.31 (d, J=6.11 Hz, 6H). MS m/e 364.0 (M+H).

### Example 14

### (2′,4′-Dimethyl-[4,5′]bithiazolyl-2-yl)-(2-trifluoromethoxy-phenyl)-amine

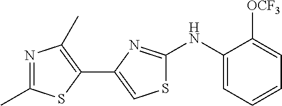

The title compound was prepared using (2-trifluoromethoxy-phenyl)-thiourea (intermediate 13, step b) in place of (2-isopropoxy-phenyl)-thiourea and 2-bromo-1-(2,4-dimethyl-thiazol-5-yl)-ethanone.HBr (intermediate 3) in place of N-[5-(2-bromo-acetyl)-4-methyl-thiazol-2-yl]-acetamide.HBr according to the procedure of example 1 (reaction time 1 d). ¹H NMR (400 MHz, DMSO-d₆) δ 10.14 (s, 1H), 8.61 (dd, J=1.47, 8.56 Hz, 1H), 7.36-7.42 (m, 2H), 7.06-7.12 (m, 1H), 7.04 (s, 1H), 2.60 (s, 3H), 2.52 (s, 3H). MS m/e 372.0 (M+H).

### Example 15

### (2-Isopropoxy-phenyl)-(4-pyridin-3-yl-thiazol-2-yl)-amine

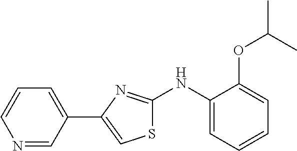

The title compound was prepared using 2-bromo-1-pyridin-3-yl-ethanone.HBr in place of N-[5-(2-bromo-acetyl)-4-methyl-thiazol-2-yl]-acetamide.HBr as described in example 1 (reaction time 22 h). ¹H NMR (300 MHz, DMSO-d₆) δ ppm 9.40 (s, 1H), 9.12 (d, J=1.9 Hz, 1H), 8.44-8.53 (m, 2H), 8.21-8.27 (m, 1H), 7.50 (s, 1H), 7.45 (dd, J=7.9, 4.9 Hz, 1H), 6.92-7.09 (m, 3H), 4.66 (sept, J=6.2 Hz, 1H), 1.32 (d, J=6.0 Hz, 6H). MS m/e 312.1 (M+H).

### Example 16

### (5-Fluoro-2-methoxy-phenyl)-(4-pyridin-3-yl-thiazol-2-yl)-amine.HBr

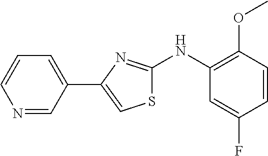

The title compound was prepared using 2-bromo-1-pyridin-3-yl-ethanone.HBr in place of 1-(2-amino-4-methyl-thiazol-5-yl)-2-bromo-ethanone as described in example 4. ¹H NMR (300 MHz, DMSO-d₆) δ ppm 10.12 (s, 1H), 9.29 (d, J=1.9 Hz, 1H), 8.88 (d, J=8.3 Hz, 1H), 8.81 (d, J=4.9 Hz, 1H), 8.49 (dd, J=11.5, 3.2 Hz, 1H), 8.06 (dd, J=8.1, 5.5 Hz, 1H), 7.89 (s, 1H), 7.05 (dd, J=8.9, 5.5 Hz, 1H), 6.80 (td, J=8.6, 3.2 Hz, 1H), 3.88 (s, 3H). MS m/e 302.1 (M+H).

### Example 17

### N²-(5-Chloro-2-methoxy-phenyl)-4′-ethyl-5-methyl-[4,5′]bithiazolyl-2,2′-diamine

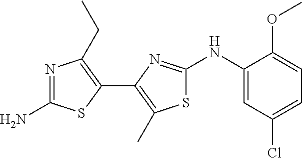

The title compound was prepared by a modification of the method described in J. Med. Chem. 2008, 51, 6044, using commercially available (5-chloro-2-methoxy-phenyl)-thiourea in place of (2-methoxy-phenyl)-thiourea according to the procedure of example 11, with purification by column chromatography (silica gel, 20-80% EtOAc-Hept). ¹H NMR (300 MHz, DMSO-d₆) δ 9.64 (s, 1H), 8.59 (d, J=2.64 Hz, 1H), 6.88-7.05 (m, 4H), 3.86 (s, 3H), 2.48 (m, partially obscured by DMSO signal), 2.24 (s, 3H), 1.17 (t, J=7.54 Hz, 3H). MS m/e 381.1; 383.1 (M+H).

### Example 18

### N-[5-Bromo-2-(5-fluoro-2-methoxy-phenylamino)-4′-methyl-[4,5′]bithiazolyl-2′-yl]-acetamide.TFA

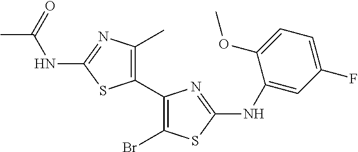

Bromine (0.006 mL, 0.109 mmol) was added to a solution of N-[2-(5-fluoro-2-methoxy-phenylamino)-4′-methyl-[4,5′]bithiazolyl-2′-yl]-acetamide.HBr (0.050 g, 0.109 mmol, example 6) in acetic acid (2 mL) at room temperature and stirred overnight. The reaction mixture was evaporated and the product was purified via reverse phase HPLC with water/acetonitrile/0.1% TFA to give the title compound. ¹H NMR (300 MHz, DMSO-d₆) δ ppm 12.21 (s, 1H) 10.12 (s, 1H) 8.32 (dd, J=11.5, 3.2 Hz, 1H) 7.02 (dd, J=9.0, 5.3 Hz, 1H) 6.76 (td, J=8.5, 3.0 Hz, 1H) 3.87 (s, 3H) 2.42 (s, 3H) 2.15 (s, 3H); MS m/e 456.9 (M+H).

### Example 19

### N-[2-(2-Cyclopropylmethoxy-phenylamino)-4′-methyl-[4,5′]bithiazolyl-2′-yl]-acetamide.TFA

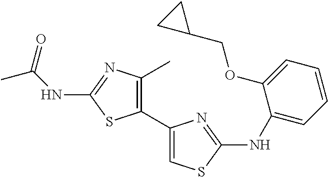

DIAD (0.059 g, 0.290 mmol) was added to a solution of N-[2-(2-hydroxy-phenylamino)-4′-methyl-[4,5′]bithiazolyl-2′-yl]-acetamide (0.050 g, 0.145 mmol, intermediate 15), triphenylphosphine (0.078 g, 0.290 mmol) and cyclopropanemethanol (0.023 mL, 0.290 mmol) in THF (2 mL) at room temperature and stirred overnight. The product was then purified via reverse phase HPLC with water/acetonitrile/0.1% TFA to give the title compound. ¹H NMR (300 MHz, MeOD) δ ppm 7.95 (d, J=7.5 Hz, 1H) 6.79-7.11 (m, 3H) 6.72 (s, 1H) 3.85 (d, J=6.8 Hz, 2H) 2.41 (s, 3H) 2.14 (s, 3H) 1.15-1.32 (m, 1H) 0.46-0.59 (m, 2H) 0.22-0.33 (m, 2H); MS m/e 401.3 (M+H).

### Example 20

### N²-(5-Bromo-2-methoxy-phenyl)-4′-methyl-[4,5′]bithiazolyl-2,2′-diamine.TFA

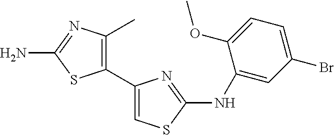

A solution of 1-(2-amino-4-methyl-thiazol-5-yl)-2-bromo-ethanone.HBr (0.030 g, 0.095 mmol, intermediate 4) and (5-bromo-2-methoxy-phenyl)-thiourea (0.025 g, 0.095 mmol, intermediate 14, step b) in ethanol (2 mL) was stirred at room temperature for 3 d. The reaction mixture was then concentrated and the residue was purified via reverse phase HPLC with water/acetonitrile/0.1% TFA to give the title compound. ¹H NMR (300 MHz, DMSO-d₆) δ 9.97 (s, 1H), 8.73 (d, J=2.64 Hz, 1H), 7.12 (dd, J=2.64, 8.67 Hz, 1H), 6.96-7.06 (m, 1H), 6.93 (s, 1H), 3.87 (s, 3H), 2.41 (s, 3H); MS m/e 396.7, 398.9 (M+H).

### Example 21

### N-[2-(5-Bromo-2-methoxy-phenylamino)-4′-methyl-[4,5′]bithiazolyl-2′-yl]-acetamide.HBr

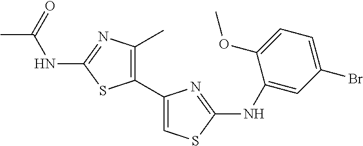

The title compound was prepared using (5-bromo-2-methoxy-phenyl)-thiourea (intermediate 14, step b) in place of (2-hydroxy-phenyl)-thiourea according to the procedure described intermediate 15 (reaction time 3 d). ¹H NMR (300 MHz, DMSO-d₆) δ ppm 12.08 (s, 1H) 9.91 (s, 1H) 8.83 (d, J=2.3 Hz, 1H) 7.06-7.19 (m, 1H) 6.85-7.03 (m, 2H) 3.88 (s, 3H) 2.52 (s, 3 H) 2.14 (s, 3H); MS m/e 438.7 (M+H).

### Example 22

### N²-(2-Methoxy-phenyl)-4′-trifluoromethyl-[4,5′]bithiazolyl-2,2′-diamine.TFA

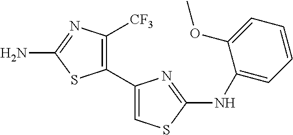

The title compound was prepared using 1-(2-amino-4-trifluoromethyl-thiazol-5-yl)-2-bromo-ethanone (intermediate 6, step c) and commercially available (2-methoxy-phenyl)-thiourea in place of 1-(2-amino-4-methyl-thiazol-5-yl)-2-bromo-ethanone.HBr and (5-bromo-2-methoxy-phenyl)-thiourea, respectively, according to the procedure described in example 20. ¹H NMR (300 MHz, MeOD, CHLOROFORM-d) δ 7.95 (dd, J=1.88, 7.54 Hz, 1H), 6.83-7.05 (m, 3H), 6.77 (s, 1H), 3.89 (s, 3H); MS m/e 416.0 (M+H).

### Example 23

### N-[2-(2,4-Dimethoxy-phenylamino)-4′-methyl-[4,5′]bithiazolyl-2′-yl]-acetamide.TFA

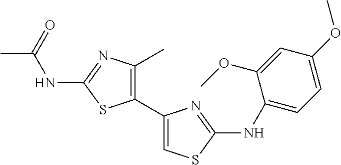

A solution of N-[5-(2-bromo-acetyl)-4-methyl-thiazol-2-yl]-acetamide.HBr (0.024 g, 0.067 mmol, intermediate 5) and commercially available 1-(2,4-dimethoxyphenyl)-2-thiourea (0.014 g, 0.067 mmol) in ethanol was heated in the microwave at 80° C. for 10 minutes. The reaction mixture was then evaporated and was purified via reverse phase HPLC with water/acetonitrile/0.1% TFA to give the title compound. ¹H NMR (300 MHz, MeOD) δ 7.76 (d, J=8.67 Hz, 1H), 6.75 (s, 1H), 6.66 (d, J=2.64 Hz, 1H), 6.58 (dd, J=2.64, 8.67 Hz, 1H), 3.89 (s, 3H), 3.82 (s, 3H), 2.46 (s, 3H), 2.23 (s, 3H); MS m/e 391.1 (M+H).

### Example 24

### (4-Pyridin-3-yl-thiazol-2-yl)-[2-(thiophen-2-ylmethoxy)-phenyl]-amine.TFA

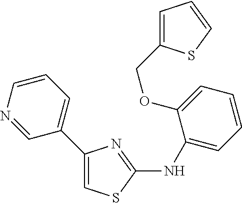

DIAD (0.041 g, 0.204 mmol) was added to a solution of 2-(4-pyridin-3-yl-thiazol-2-ylamino)-phenol (0.050 g, 0.186 mmol, intermediate 16), 2-(hydroxymethyl)thiophene (0.019 mL, 0.204 mmol) and triphenylphosphine (0.054 g, 0.204 mmol) in THF (2 mL) and the mixture was stirred at room temperature for 1 hour. The reaction mixture was evaporated and the crude mixture was purified via column chromatography with heptanes: ethyl acetate. The product was then purified via reverse phase HPLC with water/acetonitrile/0.1% TFA to give the title compound. ¹H NMR (300 MHz, MeOD) δ ppm 9.31 (d, J=1.9 Hz, 1H) 9.06 (dd, J=8.3, 1.5 Hz, 1H) 8.73 (d, J=5.3 Hz, 1H) 8.21-8.37 (m, 1H) 8.10 (dd, J=8.3, 6.4 Hz, 1H) 7.64 (s, 1H) 7.33-7.46 (m, 1H) 7.10-7.26 (m, 2H) 6.93-7.10 (m, 3H) 5.42 (s, 2H); MS m/e 366.1 (M+H).

### Example 25

### (2-Propoxy-phenyl)-(4-pyridin-3-yl-thiazol-2-yl)-amine

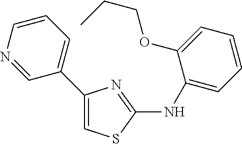

DIAD (0.041 g, 0.204 mmol) was added to a solution of 2-(4-pyridin-3-yl-thiazol-2-ylamino)-phenol (0.050 g, 0.186 mmol, intermediate 16), 1-propanol (0.012 mL, 0.204 mmol) and triphenylphosphine (0.054 g, 0.204 mmol) in THF (2 mL) and the mixture was stirred at room temperature for 2 hours. The reaction mixture was evaporated and the crude mixture was purified via column chromatography with heptanes: ethyl acetate. ¹H NMR (300 MHz, DMSO-d₆) δ 9.48 (s, 1H), 9.12 (d, J=1.51 Hz, 1H), 8.50 (dd, J=1.51, 4.90 Hz, 1H), 8.38-8.45 (m, 1H), 8.23 (dt, J=2.02, 8.01 Hz, 1H), 7.49 (s, 1H), 7.45 (dd, J=4.90, 7.91 Hz, 1H), 6.95-7.07 (m, 3H), 4.02 (t, J=6.78 Hz, 2H), 1.74-1.88 (m, 2H), 1.00 (t, J=7.54 Hz, 3H). MS m/e 312.1 (M+H).

### Example 26

### (2-Methoxy-phenyl)-(4′-methyl-2′-pyrazin-2-yl-[4,5′]bithiazolyl-2-yl)-amine.TFA

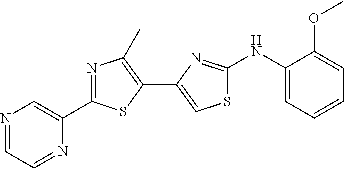

A mixture of commercially available 2-bromo-1-(4-methyl-2-pyrazin-2-yl-thiazol-5-yl)-ethanone (44 mg, 0.148 mmol) and commercially available (2-methoxy-phenyl)-thiourea (27 mg, 0.148 mmol) in EtOH was heated at 100° C. for 40 min. After cooling, the precipitate was filtered and washed with EtOAc. The solid was purified via reverse phase HPLC with water/acetonitrile/0.1% TFA to give the title compound. ¹H NMR (400 MHz, MeOD) δ 9.35 (d, J=1.47 Hz, 1H), 8.62-8.66 (m, 2H), 8.18 (d, J=7.83 Hz, 1H), 7.02-7.16 (m, 4H), 3.95 (s, 3H), 2.72 (s, 3H); MS m/e 382.4 (M+H).

### Example 27

### (5-Bromo-2-methoxy-phenyl)-(2′,4′-dimethyl-[4,5′]bithiazolyl-2-yl)-amine.TFA

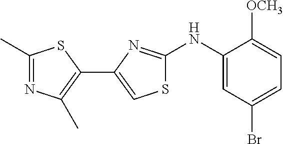

A mixture of 2-bromo-1-(2,4-dimethyl-thiazol-5-yl)-ethanone.HBr (2.41 g, 7.66 mmol, intermediate 3) and (5-bromo-2-methoxy-phenyl)-thiourea (2 g, 7.66 mmol, intermediate 14, step b) in EtOH (20 mL) was stirred at room temperature for 24 h. The mixture was filtered, washed sequentially with EtOH and heptanes, and air-dried. The crude product was further purified by reverse phase HPLC with water/acetonitrile/0.1% TFA to afford the title compound. ¹H NMR (400 MHz, CHLOROFORM-d) δ 8.48 (d, J=2.20 Hz, 1H), 7.71 (s, 1H), 7.08 (dd, J=2.20, 8.56 Hz, 1H), 6.75 (d, J=8.56 Hz, 1H), 6.62 (s, 1H), 3.90 (s, 3H), 2.64 (s, 3H), 2.68 (s, 3H); MS m/e 396.0 (M+H).

### Example 28

### (2-Methoxy-phenyl)-[4-(6-methoxy-pyridin-3-yl)-thiazol-2-yl]-amine.TFA

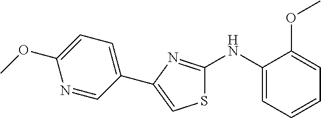

A mixture of commercially available 1-(6-methoxy-pyridin-3-yl)-ethanone (41.4 mg, 0.274 mmol) and hydroxyl(tosyloxy)-iodobenzene (118 mg, 0.302 mmol) in CH₃CN was heated at 85° C. for 3 h and concentrated. To the residue was added (2-methoxy-phenyl)-thiourea (50 mg, 0.274 mmol) and EtOH (1.5 mL) and the mixture was heated at 100° C. for 1 h. The reaction mixture was purified via reverse phase HPLC with water/acetonitrile/0.1% TFA to give the title compound. ¹H NMR (400 MHz, MeOD) δ 8.49 (d, J=2.20 Hz, 1H), 8.08 (dd, J=2.45, 8.80 Hz, 1H), 7.92 (dd, J=1.34, 7.95 Hz, 1H), 6.82-7.11 (m, 5H), 3.89 (s, 3H), 3.81 (s, 3H); MS m/e 314.1 (M+H).

### Example 29

### (2-Methoxy-phenyl)-[4-(4-methyl-pyridin-3-yl)-thiazol-2-yl]-amine.TFA

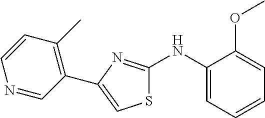

The title compound was prepared using 1-(4-methyl-pyridin-3-yl)-ethanone in place of 1-(6-methoxy-pyridin-3-yl)-ethanone according to the procedure described in example 28. ¹H NMR (400 MHz, MeOD) δ 8.62 (s, 1H), 8.24 (d, J=5.14 Hz, 1H), 8.09-8.12 (m, 1H), 7.25 (d, J=5.13 Hz, 1H), 6.82-6.93 (m, 4H), 3.82 (s, 3H), 2.47 (s, 3H); MS m/e 298.0 (M+H).

### Example 30

### (2-Methoxy-phenyl)-[4-(4-methyl-pyridin-2-yl)-thiazol-2-yl]-amine.TFA

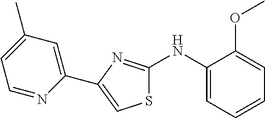

The title compound was prepared using 1-(5-methyl-pyridin-2-yl)-ethanone in place of 1-(6-methoxy-pyridin-3-yl)-ethanone according to the procedure described in example 28. ¹H NMR (400 MHz, CHLOROFORM-d) δ 8.46 (d, J=4.65 Hz, 1H), 8.04 (br. s., 1H), 7.87 (s, 2H), 7.44 (s, 1H), 6.98-7.07 (m, 3H), 6.93 (dd, J=1.59, 7.70 Hz, 1H), 3.93 (s, 3H), 2.43 (s, 3H); MS m/e 297.9 (M+H).

### Example 31

### N-[4′-Methyl-2-(2-trifluoromethoxy-phenylamino)-[4,5′]bithiazolyl-2′-yl]-acetamide.HCl

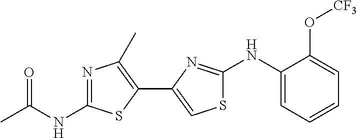

A mixture of (2-trifluoromethoxy-phenyl)-thiourea (0.33 g, 1.4 mmol, intermediate 13, step b) and N-[5-(2-bromo-acetyl)-4-methyl-thiazol-2-yl]-acetamide.HBr (0.4 g, 1.1 mmol, intermediate 5) in EtOH (20 mL) was heated to reflux overnight. The mixture was concentrated and the residue was purified by reverse phase HPLC. A solution of the purified product in THF was added to a 1 N solution of HCl in Et₂O, and the precipitated solid was collected by vacuum filtration to yield the title compound. ¹H NMR (400 MHz, DMSO-d₆) δ 12.09 (br. s., 1H), 10.12 (s, 1H), 8.60 (d, J=7.83 Hz, 1H), 7.35-7.43 (m, 2H), 7.09 (t, J=7.21 Hz, 1H), 6.98 (s, 1H), 2.48 (s, 3H), 2.14 (s, 3H). MS m/e 415.0 (M+H).

### Example 32

### N-[4′-Methyl-2-(2-trifluoromethyl-phenylamino)-[4,5′]bithiazolyl-2′-yl]-acetamide.TFA

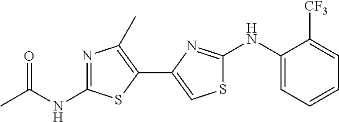

The title compound was prepared using commercially available (2-trifluoromethyl-phenyl)-thiourea in place of (2-trifluoromethoxy-phenyl)-thiourea according to the procedure of example 31, except that the HPLC-purified sample was not converted to the HCl salt. ¹H NMR (400 MHz, DMSO-d₆) δ 12.05 (br. s., 1H), 9.60 (br. s., 1H), 8.03 (d, J=8.31 Hz, 1H), 7.74 (d, J=7.83 Hz, 1H), 7.69 (t, J=7.82 Hz, 1H), 7.36 (t, J=7.46 Hz, 1H), 6.90 (s, 1H), 2.43 (s, 3H), 2.12 (s, 3H). MS m/e 399.0 (M+H).

### Example 33

### N-[4′-Methyl-2-(2-methylsulfanyl-phenylamino)-[4,5′]bithiazolyl-2′-yl]-acetamide.HCl

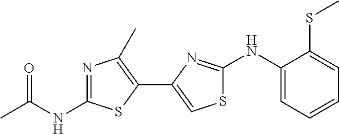

The title compound was prepared using commercially available (2-methylsulfanyl-phenyl)-thiourea in place of (2-trifluoromethoxy-phenyl)-thiourea according to the procedure of example 31. ¹H NMR (400 MHz, DMSO-d₆) δ 12.06 (br. s., 1H), 9.51 (br. s., 1H), 7.76 (dd, J=1.71, 7.58 Hz, 1H), 7.38 (dd, J=1.83, 7.46 Hz, 1H), 7.17-7.26 (m, 2H), 6.81 (s, 1H), 2.44 (s, 3H), 2.42 (s, 3H), 2.13 (s, 3H). MS m/e 377.1 (M+H).

### Example 34

### N-[2-(2-Methoxy-phenylamino)-4′-methyl-[4,5′]bithiazolyl-2′-yl]-methanesulfonamide

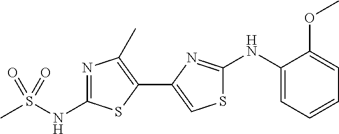

Methanesulfonyl chloride (0.144 g, 1.26 mmol) was added dropwise via syringe to an ice-cold mixture of N²-(2-methoxy-phenyl)-4′-methyl-[4,5′]bithiazolyl-2,2′-diamine (0.2 g, 0.63 mmol, example 63) and DMAP (0.154 g, 1.26 mmol) in CH₂Cl₂ (6 mL). The mixture was stirred at room temperature for 12 h. The reaction mixture was filtered to collect a grey precipitate. The solid was stirred for 20 min in sat. aq. NaHCO₃ (20 mL), and the precipitate was collected by vacuum filtration, washed with water, and lyophilized to afford the title compound. ¹H NMR (400 MHz, DMSO-d₆) δ 12.50 (br. s., 1H), 9.66 (s, 1H), 8.25-8.31 (m, 1H), 7.02-7.07 (m, 1H), 6.91-7.02 (m, 2H), 6.84 (s, 1H), 3.86 (s, 3H), 2.90 (s, 3H), 2.38 (s, 3H). MS m/e 397.0 (M+H).

### Example 35

### N²-(2-Methoxy-phenyl)-4′,N^{2′}-dimethyl-[4,5′]bithiazolyl-2,2′-diamine

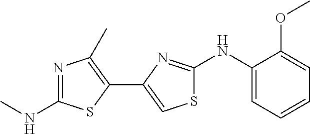

A mixture of commercially available (2-methoxy-phenyl)-thiourea (1.7 g, 9.3 mmol), 2-bromo-1-(4-methyl-2-methylamino-thiazol-5-yl)-ethanone.HBr (3.5 g, 10.6 mmol, intermediate 7, step b) and Et₃N (4 mL, 28.7 mmol) in EtOH (60 mL) was stirred at room temperature overnight. The mixture was concentrated and the residue was purified by reverse phase HPLC (18-48% CH₃CN—H₂O, 0.1% TFA). The HPLC eluant was concentrated and the residue was stirred in sat.

aq. NaHCO₃. The mixture was filtered to afford the title compound. ¹H NMR (400 MHz, DMSO-d₆) δ 10.15 (br. s., 1H), 9.80 (s, 1H), 8.24 (dd, J=1.22, 7.83 Hz, 1H), 6.97-7.08 (m, 3H), 6.91-6.97 (m, 1H), 3.86 (s, 3H), 3.08 (br. s., 3H), 2.48 (s, 3H). MS m/e 333.0 (M+H).

### Example 36

### N-[2-(2-Methoxy-phenylamino)-4′-methyl-[4,5′]bithiazolyl-2′-yl]-N-methyl-acetamide.TFA

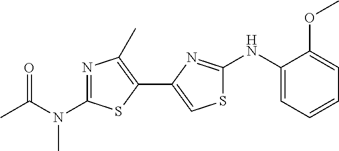

Pyridine (0.1 mL, 1.2 mmol) and acetyl chloride (0.1 mL, 1.4 mmol) were added in sequence to an ice-cold solution of N²-(2-methoxy-phenyl)-4′,N²-dimethyl-[4,5′]bithiazolyl-2,2′-diamine.TFA (0.1 g, 0.30 mmol, example 35) in a mixture of THF (9 mL) and CH₂Cl₂ (6 mL). The mixture was stirred at 0° C. for 2 h, then at room temperature overnight. Water was added and the mixture was extracted with ethyl acetate. The organic phase was dried (Na₂SO₄), filtered, and concentrated and the residue was purified by reverse phase HPLC (40-70% CH₃CN—H₂O, 0.1% TFA) to afford the title compound. MS m/e 375.0 (M+H).

### Example 37

### N^{2′}-Isopropyl-N²-(2-methoxy-phenyl)-4′-methyl-[4,5′]bithiazolyl-2,2′-diamine.HCl

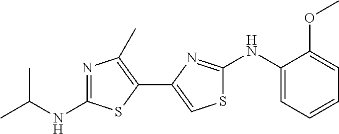

Acetic acid (1.8 mL) was added to a solution of commercially available N²-(2-methoxy-phenyl)-4′-methyl-[4,5′]bithiazolyl-2,2′-diamine (0.3 g, 0.94 mmol, example 63) in acetone (18 mL) and the mixture was stirred at room temperature for 20 min before addition of NaBH₃CN (0.59 g, 0.94 mmol). The resulting mixture was heated at reflux for 12 h, then was quenched by addition of 1 N aq. NaOH. The mixture was extracted with CH₂Cl₂ and the organic extracts were washed with water. The organic phase was dried (Na₂SO₄), filtered, and concentrated. The residue was purified by reverse phase HPLC (40-70% CH₃CN—H₂O, 0.1% TFA). The eluant was concentrated and the residue was dissolved in THF and added to 1 N HCl in Et₂O. The precipitate was collected by filtration and lyophilized to yield the title compound. ¹H NMR (400 MHz, DMSO-d₆) δ 10.25 (br. s., 1H), 9.80 (s, 1H), 8.25 (dd, J=1.47, 7.83 Hz, 1H), 7.04-7.08 (m, 1H), 6.98-7.03 (m, 2H), 6.90-6.96 (m, 1H), 4.07-4.27 (m, 1H), 3.86 (s, 3H), 2.49 (s, 3H), 1.26 (d, J=6.36 Hz, 6H). MS m/e 361.2 (M+H).

### Example 38

### N^{2′}-Ethyl-N^{2′}-(2-methoxy-phenyl)-4′-methyl-[4,5′]bithiazolyl-2,2′-diamine.HCl

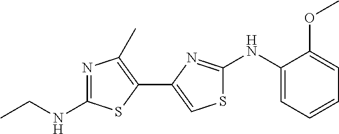

LiAlH₄ (0.194 g, 5.1 mmol) was added to a solution of commercially available N-[2-(2-methoxy-phenylamino)-4′-methyl-[4,5′]bithiazolyl-2′-yl]-acetamide (0.46 g, 1.28 mmol, example 60) in THF (20 mL). The mixture was heated at reflux for 12 h, cooled to room temperature, quenched by addition of water (3 mL), and concentrated. The residue was purified by preparative TLC, and the resulting free base was dissolved in THF and added to 1 N HCl in Et₂O. The precipitate was collected by vacuum filtration to yield the title compound. ¹H NMR (400 MHz, DMSO-d₆) δ 10.16 (br. s., 1H), 9.79 (s, 1H), 8.25 (d, J=7.83 Hz, 1H), 6.98-7.09 (m, 3H), 6.91-6.97 (m, 1H), 3.86 (s, 3H), 3.43-3.55 (m, 2H), 2.48 (s, 3H), 1.24 (t, J=7.21 Hz, 3H). MS m/e 347.1 (M+H).

### Example 39

### N-[2-(2-Chloro-phenylamino)-4′-methyl-[4,5′]bithiazolyl-2′-yl]-acetamide.HCl

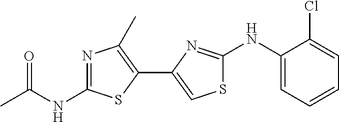

The title compound was prepared by a modification of the procedure reported in WO 2005/068444, using commercially available (2-chloro-phenyl)-thiourea in place of (2-trifluoromethoxy-phenyl)-thiourea according to the procedure of example 31. ¹H NMR (400 MHz, DMSO-d₆) δ 12.08 (br. s., 1H), 9.78 (s, 1H), 8.32 (d, J=8.31 Hz, 1H), 7.47-7.52 (m, 1H), 7.32-7.39 (m, 1H), 7.05-7.11 (m, 1H), 6.95 (s, 1H), 2.46 (s, 3H), 2.13 (s, 3H). MS m/e 365.1 (M+H).

### Example 40

### N-[2-(2-Fluoro-phenylamino)-4′-methyl-[4,5′]bithiazolyl-2′-yl]acetamide.TFA

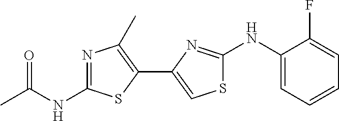

The title compound was prepared using commercially available (2-fluoro-phenyl)-thiourea in place of (2-trifluoromethoxy-phenyl)-thiourea according to the procedure of example 31, except that the HPLC-purified sample was not converted to the HCl salt. ¹H NMR (400 MHz, DMSO-d₆) δ 12.09 (s, 1H), 10.14 (s, 1H), 8.41-8.48 (m, 1H), 7.17-7.31 (m, 2H), 6.99-7.06 (m, 1H), 6.94 (s, 1H), 2.48 (s, 3H), 2.14 (s, 3H). MS m/e 349.1 (M+H).

### Example 41

### (5-Fluoro-2-methoxy-phenyl)-[4-(1-methyl-1H-benzoimidazol-5-yl)-thiazol-2-yl]-amine

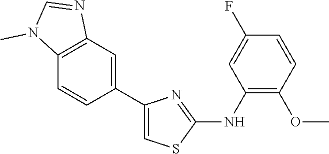

A mixture of 2-bromo-1-(1-methyl-1H-benzoimidazol-5-yl)-ethanone.HBr (50 mg, 0.15 mmol, intermediate 17, step c) and (5-fluoro-2-methoxy-phenyl)-thiourea (27.0 mg, 0.135 mmol, intermediate 9, step b) in EtOH (1.5 mL) was stirred at room temperature for 24 h. The mixture was basified with 2 N NH₃/MeOH and silica gel (300 mesh, ˜1 g) was added. The resulting suspension was concentrated and purified through solid loading on column chromatography (3%-10% MeOH/DCM) to yield the title compound as a white solid. ¹H NMR (400 MHz, CHLOROFORM-d) δ 8.30 (s, 1H), 8.23 (dd, J=2.93, 10.76 Hz, 1H), 7.77-8.05 (m, 3H), 7.39 (d, J=8.56 Hz, 1H), 6.86 (s, 1H), 6.78 (dd, J=4.89, 8.80 Hz, 1H), 6.64 (dd, J=3.06, 8.19 Hz, 1H), 3.88 (s, 3H), 3.83 (s, 3H); MS m/e 355.1 (M+H).

### Example 42

### (5-Fluoro-2-methoxy-phenyl)-[4-(2,3,5-trimethyl-3H-imidazol-4-yl)-thiazol-2-yl]-amine

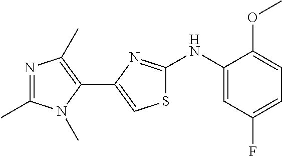

A mixture of (5-fluoro-2-methoxy-phenyl)-thiourea (24.7 mg, 0.123 mmol, intermediate 9, step b), 2-bromo-1-(2,3,5-trimethyl-3H-imidazol-4-yl)-ethanone (28.5 mg, 0.123 mmol, intermediate 18, step e), and EtOH (1.0 mL) was stirred at room temperature for 1 d. The mixture was partitioned between sat. aq. NaHCO₃ and EtOAc. The aq. phase was extracted with EtOAc. The organic phase was dried (Na₂SO₄), filtered, and concentrated and the residue was purified by flash column chromatography (silica gel, 0-3% MeOH—CH₂Cl₂), affording the title compound as a white powder. ¹H NMR (400 MHz, DMSO-d₆) δ 9.89 (s, 1H), 8.41 (dd, J=3.06, 11.86 Hz, 1H), 7.00 (dd, J=5.26, 8.93 Hz, 1H), 6.84 (s, 1H), 6.72 (td, J=3.18, 8.56 Hz, 1H), 3.86 (s, 3H), 3.61 (s, 3H), 2.29 (s, 3H), 2.17 (s, 3H). MS m/e 333.1 (M+H).

### Example 43

### (2-Methoxy-phenyl)-[4-(5-phenyl-thiophen-2-yl)-thiazol-2-yl]-amine.TFA

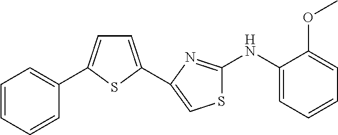

A mixture of 2-bromo-1-(5-phenyl-thiophen-2-yl)-ethanone (77 mg, 0.274 mmol) and hydroxyl(tosyloxy)-iodobenzene (118 mg, 0.302 mmol) in CH₃CN was heated at 85° C. for 3 h and concentrated. To the residue was added (2-methoxy-phenyl)-thiourea (50 mg, 0.274 mmol) and EtOH (1.5 mL) and the mixture was heated at 100° C. for 1 h. The reaction mixture was purified via reverse phase HPLC with water/acetonitrile/0.1% TFA to give the title compound. ¹H NMR (400 MHz, MeOD) δ 7.85 (dd, J=1.59, 7.95 Hz, 1H), 7.69 (d, J=7.58 Hz, 2H), 7.40-7.46 (m, 4H), 7.27-7.37 (m, 2H), 7.05-7.21 (m, 3H), 3.96 (s, 3H); MS m/e 365.1 (M+H).

### Example 44

### [4-(2,4-Dimethyl-oxazol-5-yl)-thiazol-2-yl]-(2-methoxy-phenyl)-amine.TFA

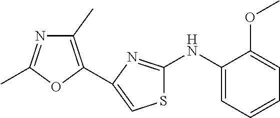

A mixture of 1-(2,4-dimethyl-oxazol-5-yl)-ethanone (38 mg, 0.274 mmol) and hydroxyl(tosyloxy)-iodobenzene (118 mg, 0.302 mmol) in CH₃CN was heated at 85° C. for 3 h and concentrated. To the residue was added (2-methoxy-phenyl)-thiourea (50 mg, 0.274 mmol) and EtOH (1.5 mL) and the mixture was heated at 100° C. for 1 h. The reaction mixture was purified via reverse phase HPLC with water/acetonitrile/0.1% TFA to give the title compound. ¹H NMR (400 MHz, MeOD) δ 8.15 (d, J=7.83 Hz, 1H), 6.76-6.93 (m, 4H), 3.82 (s, 3H), 2.38 (d, J=2.20 Hz, 6H); MS m/e 302.1 (M+H).

### Example 45

### [4-(3,5-Dimethyl-thiophen-2-yl)-thiazol-2-yl]-(2-methoxy-phenyl)-amine.TFA

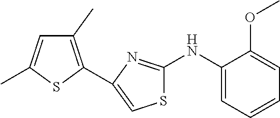

A mixture of 1-(3,5-dimethyl-thiophen-2-yl)-ethanone (42 mg, 0.274 mmol) and hydroxyl(tosyloxy)-iodobenzene (118 mg, 0.302 mmol) in CH₃CN was heated at 85° C. for 3 h and concentrated. To the residue was added (2-methoxy-phenyl)-thiourea (50 mg, 0.274 mmol) and EtOH (1.5 mL) and the mixture was heated at 100° C. for 1 h. The reaction mixture was purified via reverse phase HPLC with water/acetonitrile/0.1% TFA to give the title compound. ¹H NMR (400 MHz, MeOD) δ 8.04 (dd, J=1.59, 7.95 Hz, 1H), 6.98-7.16 (m, 4H), 6.59-6.63 (m, 1H), 3.94 (s, 3H), 2.45 (s, 3H), 2.34 (s, 3H); MS m/e 317.1 (M+H).

The following examples are commercially available:

| Example | Structure | Chemical name | Source |
|---|---|---|---|
| 46 | 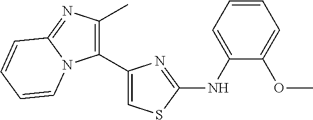 | (2-Methoxy- phenyl)-[4-(2- methyl- imidazo[1,2- a]pyridin-3-yl)- thiazol-2-yl]- amine | Specs and Biospecs |
| 47 | 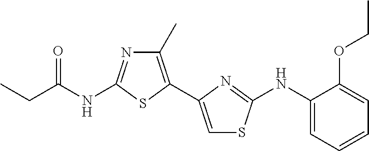 | N-[2-(2-Ethoxy- phenylamino)-4′- methyl- [4,5′]bithiazolyl- 2′-yl]- propionamide | Specs and Biospecs |
| 48 | 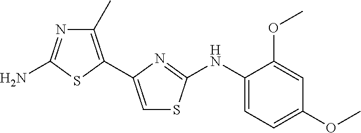 | N²-(2,4- Dimethoxy- phenyl)-4′- methyl- [4,5′]bithiazolyl- 2,2′-diamine | ASINEX |
| 49 | 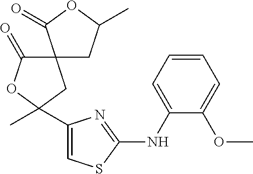 | 3-[2-(2-Methoxy- phenylamino)- thiazol-4-yl]-3,8- dimethyl-2,7- dioxa- spiro[4.4]nonane- 1,6-dione | ChemBridge |
| 50 | 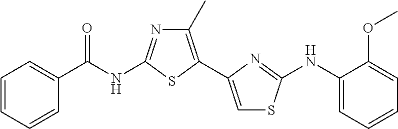 | N-[2-(2-Methoxy- phenylamino)-4′- methyl- [4,5′]bithiazolyl- 2′-yl]-benzamide | ASINEX |
| 51 | 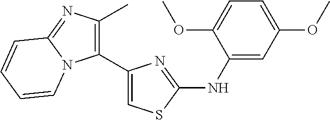 | (2,5-Dimethoxy- phenyl)-[4-(2- methyl- imidazo[1,2- a]pyridin-3-yl)- thiazol-2-yl]- amine | Specs and Biospecs |
| 52 | 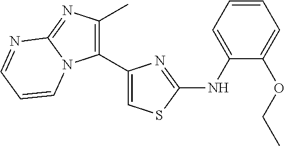 | (2-Ethoxy- phenyl)-[4-(2- methyl- imidazo[1,2- a]pyrimidin-3-yl)- thiazol-2-yl]- amine | Specs and Biospecs |
| 53 | 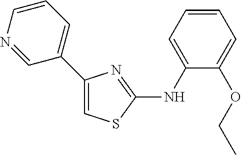 | (2-Ethoxy- phenyl)-(4- pyridin-3-yl- thiazol-2-yl)- amine | ASINEX |
| 54 | 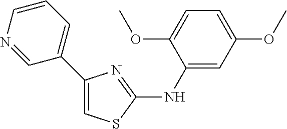 | (2,5-Dimethoxy- phenyl)-(4- pyridin-3-yl- thiazol-2-yl)- amine | Specs and Biospecs |
| 55 | 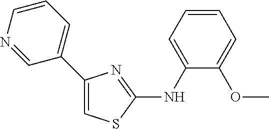 | (2-Methoxy- phenyl)-(4- pyridin-3-yl- thiazol-2-yl)- amine | ASINEX |
| 56 | 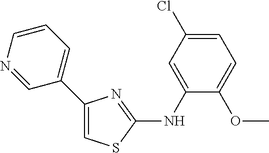 | (5-Chloro-2- methoxy-phenyl)- (4-pyridin-3-yl- thiazol-2-yl)- amine | ASINEX |
| 57 | 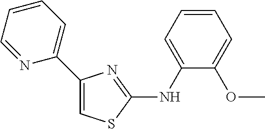 | (2-Methoxy- phenyl)-(4- pyridin-2-yl- thiazol-2-yl)- amine | ASINEX |
| 58 | 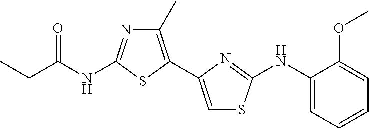 | N-[2-(2-Methoxy- phenylamino)-4′- methyl- [4,5′]bithiazolyl- 2′-yl]- propionamide | ChemBridge |
| 59 | 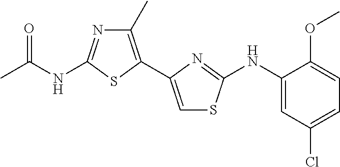 | N-[2-(5-Chloro-2- methoxy- phenylamino)-4′- methyl- [4,5′]bithiazolyl- 2′-yl]-acetamide | ASINEX |
| 60 | 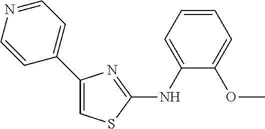 | (2-Methoxy- phenyl)-(4- pyridin-4-yl- thiazol-2-yl)- amine | ASINEX |
| 61 | 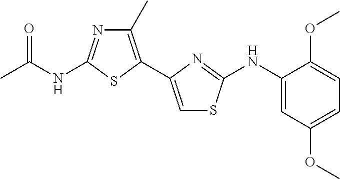 | N-[2-(2,5- Dimethoxy- phenylamino)-4′- methyl- [4,5′]bithiazolyl- 2′-yl]-acetamide | ChemBridge |
| 62 | 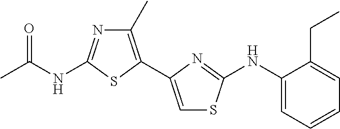 | N-[2-(2-Ethyl- phenylamino)-4′- methyl- [4,5′]bithiazolyl- 2′-yl]-acetamide | ChemBridge |
| 63 | 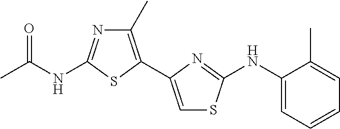 | N-(4′-Methyl-2-o- tolylamino- [4,5′]bithiazolyl- 2′-yl)-acetamide | ChemBridge |
| 64 | 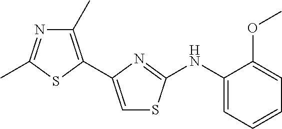 | (2′-4′-Dimethyl- [4,5′]bithiazolyl- 2-yl)-(2-methoxy- phenyl)-amine | ChemBridge |
| 65 | 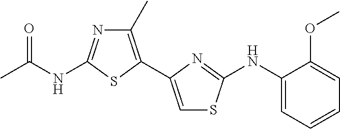 | N-[2-(2-Methoxy- phenylamino)-4′- methyl- [4,5′]bithiazolyl- 2′-yl]-acetamide | ChemBridge |
| 66 | 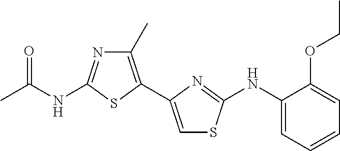 | N-[2-(2-Ethoxy- phenylamino)-4′- methyl- [4,5′]bithiazolyl- 2′-yl]-acetamide | ChemBridge |
| 67 | 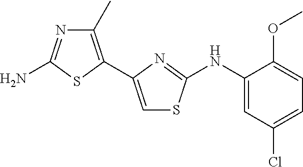 | N²-(5-Chloro-2- methoxy-phenyl)- 4′-methyl- [4,5′]bithiazolyl- 2,2′-diamine | ChemBridge |
| 68 | 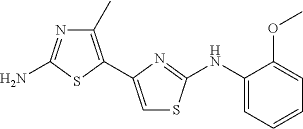 | N²-(2-Methoxy- phenyl)-4′- methyl- [4,5′]bithiazolyl- 2,2′-diamine | ASINEX |
| 69 | 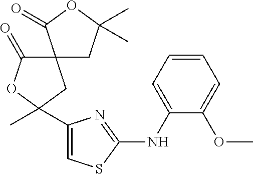 | 3-[2-(2-Methoxy- phenylamino)- thiazol-4-yl]- 3,8,8-trimethyl- 2,7-dioxa- spiro[4.4]nonane- 1,6-dione | Specs and Biospecs |

The following examples were obtained from the Johnson and Johnson corporate compound collection:

| | | Chemical |
|---|---|---|
| Example | Structure | name |
| 70 | 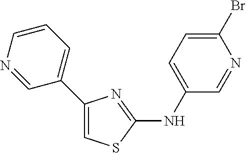 | (6-Bromo- pyridin-3- yl)-(4- pyridin-3-yl- thiazol-2- yl)-amine |
| 71 | 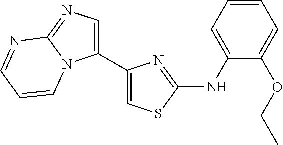 | (2-Ethoxy- phenyl)-(4- imidazo[1,2- a]pyrimidin- 3-yl-thiazol- 2-yl)-amine |
| 72 | 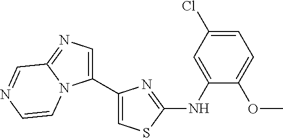 | (5-Chloro-2- methoxy- phenyl)-(4- imidazo[1,2- a]pyrazin-3- yl-thiazol-2- yl)-amine |
| 73 | 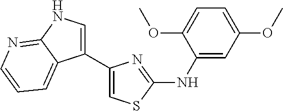 | (2,5- Dimethoxy- phenyl)-[4- (1H- pyrrolo[2,3- b]pyridin-3- yl)-thiazol- 2-yl]-amine |
| 74 | 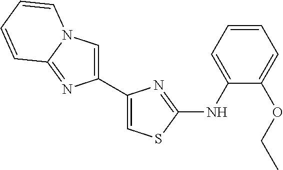 | (2-Ethoxy- phenyl)-(4- imidazo[1,2- a]pyridin-2- yl-thiazol-2- yl)-amine |
| 75 | 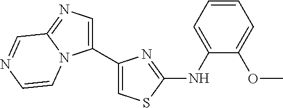 | (4- Imidazo[1,2- a]pyrazin-3- yl-thiazol-2- yl)-(2- methoxy- phenyl)- amine |
| 76 | 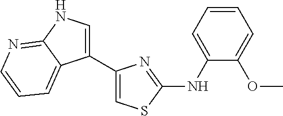 | (2-Methoxy- phenyl)-[4- (1H- pyrrolo[2,3- b]pyridin-3- yl)-thiazol- 2-yl]-amine |

### Example 70

### (6-Bromo-pyridin-3-yl)-(4-pyridin-3-yl-thiazol-2-yl)-amine

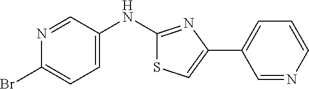

(6-Bromo-pyridin-3-yl)-(4-pyridin-3-yl-thiazol-2-yl)-amine (example 70) is synthesized by stirring roughly equimolar amounts of commercially available 2-bromo-1-pyridin-3-yl-ethanone.HBr and (6-bromo-pyridin-3-yl)-thiourea in ethanol at a temperature in the range 20-100° C. for a time period between 10 minutes and 3 days. The product is isolated by concentration of the reaction mixture and purification of the residue by reverse-phase HPLC.

The required starting material, (6-bromo-pyridin-3-yl)-thiourea, is synthesized by the general method described in Synthesis 1988, 456, by heating an approximately equimolar mixture of commercially available 3-amino-6-bromopyridine and benzoyl isothiocyanate in acetone at reflux for a time period between 15 min and 8 hours. The product from this reaction is heated to reflux in 10% aq. NaOH for between 15 min and 8 hours, providing (6-bromo-pyridin-3-yl)-thiourea, which may be isolated by filtration or extraction followed by purification by flash column chromatography using silica gel.

### Example 71

### (2-Ethoxy-phenyl)-(4-imidazo[1,2-a]pyrimidin-3-yl-thiazol-2-yl)-amine

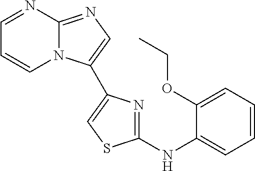

Step a: 1-Imidazo[1,2-a]pyrimidin-3-yl-ethanone (CAS 453548-59-9) is obtained from Hangzhou Chempro Tech Co., Inc., or is prepared by the method described in WO 2002/066481. 2-Bromo-1-imidazo[1,2-a]pyrimidin-3-yl-ethanone is synthesized by adding a solution of bromine (approximately 1 molar equivalent) in 1,4-dioxane to a solution of 1-imidazo[1,2-a]pyrimidin-3-yl-ethanone in 1,4-dioxane and stirring at a temperature in the range 20-100° C. for a time period between 10 minutes and 48 hours. The product is isolated as the HBr salt by filtration or as the free base by partitioning between an organic solvent, such as dichloromethane or ethyl acetate, and saturated aqueous NaHCO₃ solution, collecting the organic phase, drying over Na₂SO₄, filtering, and concentrating. The free base can be further purified by flash column chromatography on silica gel.

Step b: (2-Ethoxy-phenyl)-(4-imidazo[1,2-a]pyrimidin-3-yl-thiazol-2-yl)-amine (example 71) is synthesized by stirring roughly equimolar amounts of 2-bromo-1-imidazo[1,2-a]pyrimidin-3-yl-ethanone (example 71, step a) and commercially available 1-(2-ethoxyphenyl)-2-thiourea in ethanol at a temperature in the range 20-100° C. for a time period between 10 minutes and 3 days. The product is isolated by concentration of the reaction mixture and purification of the residue by reverse-phase HPLC.

### Example 72

### (5-Chloro-2-methoxy-phenyl)-(4-imidazo[1,2-a]pyrazin-3-yl-thiazol-2-yl)-amine

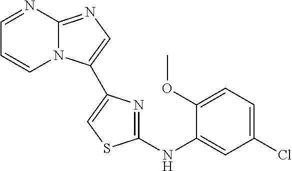

Step a: 1-Imidazo[1,2-a]pyrazin-3-yl-ethanone (CAS 78109-26-9) is obtained from Hangzhou Chempro Tech Co., Inc., or is prepared by the method described in WO 2002/066481. 2-Bromo-1-imidazo[1,2-a]pyrazin-3-yl-ethanone is synthesized using 1-imidazo[1,2-a]pyrazin-3-yl-ethanone in place of 1-imidazo[1,2-a]pyridin-3-yl-ethanone according to the procedure of example 71, step a.

Step b: (5-Chloro-2-methoxy-phenyl)-(4-imidazo[1,2-a]pyrazin-3-yl-thiazol-2-yl)-amine (example 72) is synthesized using 2-bromo-1-imidazo[1,2-a]pyrazin-3-yl-ethanone (example 72, step a) in place of 2-bromo-1-imidazo[1,2-a]pyrimidin-3-yl-ethanone and commercially available 5-chloro-2-methoxyphenylthiourea in place of 1-(2-ethoxyphenyl)-2-thiourea by the method described in example 71, step b.

### Example 73

### (2,5-Dimethoxy-phenyl)-[4-(1H-pyrrolo[2,3-b]pyridin-3-yl)-thiazol-2-yl]-amine

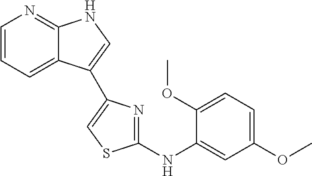

Step a: 2-Bromo-1-(1H-pyrrolo[2,3-b]pyridin-3-yl)-ethanone is synthesized using commercially available 3-acetyl-7-azaindole in place of 1-imidazo[1,2-a]pyrimidin-3-yl-ethanone according to the procedure of example 71, step a.

Step b: (2,5-Dimethoxy-phenyl)-[4-(1H-pyrrolo[2,3-b]pyridin-3-yl)-thiazol-2-yl]-amine (example 73) is synthesized using 2-bromo-1-(1H-pyrrolo[2,3-b]pyridin-3-yl)-ethanone (example 73, step a) in place of 2-bromo-1-imidazo[1,2-a]pyrimidin-3-yl-ethanone and commercially available (2,5-dimethoxyphenyl)thiourea in place of 1-(2-ethoxyphenyl)-2-thiourea by the method described in example 71, step b.

### Example 74

### (2-Ethoxy-phenyl)-(4-imidazo[1,2-a]pyridin-2-yl-thiazol-2-yl)-amine

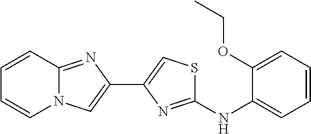

Step a: 2-Bromo-1-imidazo[1,2-a]pyridin-2-yl-ethanone is synthesized using commercially available 1-imidazo[1,2-a]pyridin-2-yl-ethanone in place of 1-imidazo[1,2-a]pyridin-3-yl-ethanone according to the procedure of example 71, step a.

Step b: (2-Ethoxy-phenyl)-(4-imidazo[1,2-a]pyridin-2-yl-thiazol-2-yl)-amine (example 74) is synthesized using 2-bromo-1-imidazo[1,2-a]pyridin-2-yl-ethanone (example 74, step a) in place of 2-bromo-1-imidazo[1,2-a]pyrimidin-3-yl-ethanone by the method described in example 71, step b.

### Example 75

### (4-Imidazo[1,2-a]pyrazin-3-yl-thiazol-2-yl)-(2-methoxy-phenyl)-amine

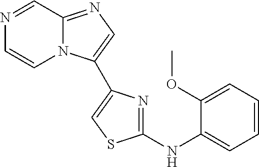

(4-Imidazo[1,2-a]pyrazin-3-yl-thiazol-2-yl)-(2-methoxy-phenyl)-amine (example 75) is synthesized using 2-bromo-1-imidazo[1,2-a]pyrazin-3-yl-ethanone (example 72, step a) in place of 2-bromo-1-imidazo[1,2-a]pyrimidin-3-yl-ethanone and commercially available 1-(2-methoxyphenyl)-2-thiourea in place of 1-(2-ethoxyphenyl)-2-thiourea by the method described in example 71, step b.

### Example 76

### (2-Methoxy-phenyl)-[4-(1H-pyrrolo[2,3-b]pyridin-3-yl)-thiazol-2-yl]-amine

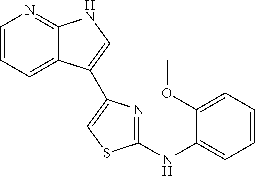

(2-Methoxy-phenyl)-[4-(1H-pyrrolo[2,3-b]pyridin-3-yl)-thiazol-2-yl]-amine (example 76) is synthesized using 2-bromo-1-(1H-pyrrolo[2,3-b]pyridin-3-yl)-ethanone (example 73, step a) in place of 2-bromo-1-imidazo[1,2-a]pyrimidin-3-yl-ethanone and commercially available 1-(2-methoxyphenyl)-2-thiourea in place of 1-(2-ethoxyphenyl)-2-thiourea by the method described in example 71, step b.

### Example 77

### N-(5-fluoro-2-methoxyphenyl)-2′,4′,5-trimethyl-[4,5′-bithiazol]-2-amine.HBr

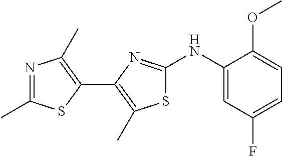

The title compound was prepared using 2-bromo-1-(2,4-dimethylthiazol-5-yl)propan-1-one (intermediate 19, step b) in place of 1-(2-amino-4-methyl-thiazol-5-yl)-2-bromo-ethanone according to the procedure of example 4 (reaction time 1 d). ¹H NMR (400 MHz, DMSO-d₆) δ 9.82 (s, 1H), 8.34 (dd, J=3.18, 11.74 Hz, 1H), 6.99 (dd, J=5.26, 8.93 Hz, 1H), 6.71 (td, J=3.06, 8.50 Hz, 1H), 3.85 (s, 3H), 2.67 (s, 3H), 2.34 (s, 3H), 2.26 (s, 3H). MS m/e 350.0 (M+H).

### Example 78

### N-(5-fluoro-2-methoxyphenyl)-4′-methyl-2′-(1-methylpiperidin-4-yl)-[4,5′-bithiazol]-2-amine.TFA

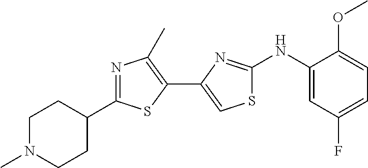

A mixture of 2-bromo-1-(4-methyl-2-(1-methylpiperidin-4-yl)thiazol-5-yl)ethanone.HBr (35.0 mg, 0.088 mmol, intermediate 20, step c), 1-(5-fluoro-2-methoxyphenyl)thiourea (17.6 mg, 0.088 mmol, intermediate 9, step b), and EtOH (1 mL) was stirred at room temperature overnight. The reaction mixture was diluted with sat. aq. NaHCO₃ and extracted with EtOAc. The organic phase was dried (Na₂SO₄), filtered, and concentrated. The crude product was purified by RP-HPLC (10-90% CH₃CN—H₂O, 0.1% TFA). ¹H NMR (400 MHz, DMSO-d₆) δ 10.01 (s, 1H), 9.40 (br. s., 1H), 8.44 (dd, J=3.06, 11.86 Hz, 1H), 7.07 (s, 1H), 7.02 (dd, J=5.14, 8.80 Hz, 1H), 6.75 (td, J=3.06, 8.50 Hz, 1H), 3.87 (s, 3H), 3.49-3.58 (m, 2H), 3.03-3.29 (m, 3H), 2.79-2.85 (m, 3H), 2.55 (s, 3H), 2.22-2.32 (m, 2H), 1.84-1.99 (m, 2H). MS m/e 419.2 (M+H).

### Example 79

### 4-(6-(4-Cyclopropylpiperazin-1-yl)pyridin-3-yl)-N-(5-fluoro-2-methoxyphenyl)thiazol-2-amine.TFA

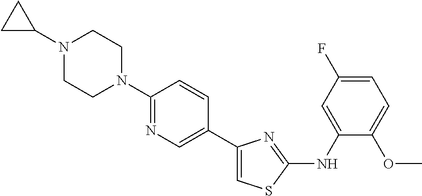

A solution of 2-bromo-1-(6-(4-cyclopropylpiperazin-1-yl)pyridin-3-yl)ethanone.HBr (0.025 g, 0.051 mmol, intermediate 21, step b) and 1-(5-fluoro-2-methoxyphenyl)thiourea (0.015 g, 0.051 mmol, intermediate 9, step b) in ethanol was stirred at room temperature overnight. The reaction mixture was then evaporated and purified via reverse phase HPLC eluting with water/acetonitrile/0.1% TFA to give the title compound. ¹H NMR (DMSO-d₆) δ: 9.92 (s, 1H), 9.40 (br. s., 1H), 8.72 (d, J=2.2 Hz, 1H), 8.56 (dd, J=11.9, 3.1 Hz, 1H), 8.10 (dd, J=8.9, 2.3 Hz, 1H), 7.27 (s, 1H), 6.92-7.21 (m, 2H), 6.72-6.84 (m, 1H), 4.40-4.64 (m, 2H), 3.87 (s, 3H), 3.48-3.74 (m, 2H), 3.24-3.48 (m, 2H), 3.04-3.24 (m, 2H), 2.78-3.04 (m, 1H), 0.93-1.08 (m, 2H), 0.78-0.93 (m, 2H).

### Compound α

### 3-(2′,4′-Dimethyl-[4,5′]bithiazolyl-2-ylamino)-4-isopropoxy-benzenesulfonamide.HBr

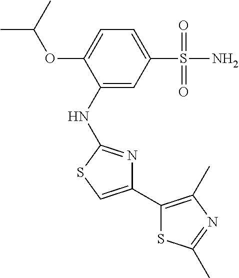

Compound α was tested in cell based, in-vitro and in-vivo assays (vide infra) The cell based, in-vitro and in-vivo activity of Compound α is provided as representative of the activity of the compounds of the present invention, but is not to be construed as limiting the invention in any way.

### Cloning, Expression and Purification

Cloning of Human proMMP9

Amino acid numbering for all human proMMP9 constructs was based on UniProtKB/Swiss-Prot P14780, full-length human matrix metalloproteinase-9 precursor, proMMP9(1-707) (SEQ ID NO:1). One construct, proMMP9(20-445) (SEQ ID NO:2), was based on the previously published crystal structure (Acta Crystallogr D Biol Crystallogr 58(Pt 7): 1182-92). The construct lacked the signal peptide at the N-terminus and also lacked the four hemopexin-like domains at the C-terminus. An N-terminal truncated construct was also designed with an N-terminus truncation after the first observable electron density in the previously published proMMP9 structure and a single amino acid was removed from the C-terminus to produce proMMP9(29-444) (SEQ ID NO:3). Other truncated constructs were also synthesized without the three fibronectin type-II domains (ΔFnII), amino acids 216-390. The ΔFnII constructs were proMMP9(29-444;ΔFnII) (SEQ ID NO:4), proMMP9(67-444;ΔFnII) (SEQ ID NO:5) and proMMP9(20-445;ΔFnII) (SEQ ID NO:6). Binding studies with the proMMP9 proteins without the FnII domains showed that compounds bound with similar affinity compared to the wild-type protein (data not shown).

In order to make the constructs with the FnII domains deleted, proMMP9(29-444;ΔFnII) (SEQ ID NO:4), proMMP9(67-444;ΔFnII) (SEQ ID NO:5) and proMMP9(20-445;ΔFnII) (SEQ ID NO:6), plasmids encoding the different proMMP9 truncations were used as templates for PCR to create two fragments of DNA corresponding to amino acid pairs including: 29-215/391-444, 67-215/391-444, and 20-215/391-445, respectively. Overlapping PCR was used to join the fragments. The 5′ primers had an Nde1 site and a start methionine and the 3′ primers had a stop codon and a Bgl2 site. The final PCR products were cloned into the TOPO TA cloning vector (Invitrogen) and the sequences were confirmed. Subsequently the vectors were digested with Nde1 and Bgl2 and the sequences were subcloned into Nde1 and BamH1 sites of the T7 expression vector pET11a (Novagen).

Expression of Truncated Forms of Human proMMP9

For expression in E. coli, all of the truncated proMMP9 constructs were transformed into BL21(DE3) RIL cells (Stratagene). Cells were initiated for an overnight culture from glycerol stocks in LB+Ampicillin (100 μg/ml) @ 37° C. shaking at 220 rpms. The overnight culture was subcultured 1:100 in LB+Ampicillin (100 ug/ml) and maintained at 37° C. shaking at 220 rpms. Samples were taken and A600 readings were monitored until an OD of 0.6 was achieved. The culture was induced with 1 mM IPTG and maintained under present growth conditions. Cultures were harvested 3 hours post induction at 6000×g for 10 min. Pellets were washed in 1×PBS with protease inhibitors and stored at −80° C.

Purification of Truncated Forms of Human proMMP9

To purify the truncated proMMP9 proteins from E. coli, cell pellets were suspended in 25 mM Na₂HPO₄ pH 7, 150 mM NaCl, 10 mL/gram cell pellet. The cells were homogenized in a Dounce homogenizer, and then processed twice through a microfluidizer (Microfluidics International Corporation, model M-110Y). The lysate was centrifuged at 32,000×g for 45 minutes at 4° C. The supernatant was discarded. The pellet was suspended in 25 mM Na₂HPO₄ pH 7, 150 mM NaCl, 10 mM DTT, 1 mM EDTA, 10 mL/gram cell pellet. The pellet was homogenized in a Dounce homogenizer, and then centrifuged at 32,000×g for 45 minutes at 4° C. The supernatant was discarded. The pellet was suspended in 7 M urea, 25 mM Tris pH 7.5, 10 mM DTT, 1 mM EDTA, 6.5 mL/gram cell pellet, and then solubilized in a Dounce homogenizer and stirred for approximately 16 hours at ambient temperature. The solubilized protein solution was adjusted to pH 7.5, centrifuged at 45,000×g, 45 minutes at 4° C., and the supernatant, containing the denatured proMMP9, was filtered to 0.8 micron. A 5 mL HiTrap Q Sepharose HP column (GE Healthcare) was prepared according to manufacturer's instructions using Buffer A: 7 M urea, 25 mM Tris pH 7.5 and Buffer B: 7 M urea, 25 mM Tris pH 7.5, 1.0 M NaCl. The protein solution was applied to the HiTrap at 2.5 mL/minute. The column was washed to baseline absorbance with approximately 3.5 CV Buffer A. The proMMP9 was eluted in a 12CV linear gradient from 0% Buffer B to 12% Buffer B. Fractions were collected, analyzed on SDS-PAGE (Novex) and pooled based on purity. The pooled protein was re-natured by drop-wise addition to a solution, stirring and at ambient temperature, of 20 mM Tris pH 7.5, 200 mM NaCl, 5 mM CaCl₂, 1 mM ZnCl₂, 0.7 M L-arginine, 10 mM reduced and 1 mM oxidized glutathione, and was stirred for approximately 16 hours at 4° C. The refolded protein was concentrated to approximately 2.5 mg/mL in Jumbo Sep centrifugal concentrators (Pall) with 10,000 MWCO membranes. The concentrated protein solution was dialyzed at 4° C. for approximately 16 hours against 20 mM Tris pH 7.5, 150 mM NaCl. The dialyzed protein solution was clarified by filtration to 0.8 micron, concentrated to 2 mg/mL as before, centrifuged at 45,000×g for 15 minutes at 4° C. and filtered to 0.2 micron. It was purified on a HiLoad 26/60 Superdex 200 column (GE Healthcare) equilibrated in 20 mM Tris pH 7.5, 200 mM NaCl. Fractions were analyzed by SDS-PAGE and pooled based on purity. The pooled protein was concentrated in a Jumbo Sep concentrator as before and centrifuged at 16,000×g for 10 minutes at 4° C. The protein concentration was determined using Bio-Rad Protein Assay (Bio-Rad Laboratories, Inc.) with bovine serum albumin as a standard. The supernatant was aliquoted, frozen in liquid nitrogen and stored at −80° C.

Full-Length Human proMMP9

Full-length proMMP9(1-707) (SEQ ID NO:1) was expressed in HEK293 cells or in COS-1 cells as a secreted protein using a pcDNA3.1 expression vector. When expressed as a secreted protein in HEK293 cells or COS-1 cells, there is cotranslational removal of the signal peptide, amino acids 1-19 of full-length proMMP9(1-707) (SEQ ID NO:1). The final purified proMMP9(1-707) (SEQ ID NO:1) protein lacks the signal peptide.

Prior to transfection with the proMMP9(1-707) (SEQ ID NO:1) construct, the HEK293 cells were suspension adapted (shake flasks) in a serum free media (Freestyle 293) supplemented with pluronic acid (F-68) at a final concentration of 0.1%. Once cells reached a density of 1.2×10⁶/mL they were transiently transfected using standard methods. Transient transfection of COS-1 cells was done in flasks with adherent cell cultures and serum free media. For both HEK293 and COS-1 cells, the conditioned media was collected for purification of the proMMP9(1-707) (SEQ ID NO:1) protein. 1.0 M HEPES pH 7.5 was added to 9 L of conditioned media for a final concentration of 50 mM. The media was concentrated to 600 mL in a Kvicklab concentrator fitted with a hollow fiber cartridge of 10,000 MWCO (GE Healthcare). This was clarified by centrifugation at 6,000×g, 15 minutes, at 4° C. and then further concentrated to 400 mL in Jumbo Sep centrifugal concentrators (Pall) with 10,000 MWCO membranes. The concentrated protein was dialyzed against 50 mM HEPES pH 7.5, 10 mM CaCl₂, 0.05% Brij 35, overnight at 4° C. and then dialysis was continued for several hours at 4° C. in fresh dialysis buffer. The dialyzed protein was centrifuged at 6,000×g, 15 minutes, at 4° C., and filtered to 0.45 micron. 12 mL of Gelatin Sepharose 4B resin (GE Healthcare) was equilibrated in 50 mM HEPES pH 7.5, 10 mM CaCl₂, 0.05% Brij 35 in a 2.5 cm diameter Econo-Column (Bio-Rad Laboratories). The filtered protein solution was loaded onto the Gelatin Sepharose resin using gravity flow at approximately 3 mL/minute. The resin was washed with 10CV 50 mM HEPES pH 7.5, 10 mM CaCl₂, 0.05% Brij 35 and eluted with 30 mL 50 mM HEPES pH 7.5, 10 mM CaCl₂, 0.05% Brij 35, 10% DMSO, collected in 5 mL fractions. Fractions containing protein, confirmed by A280 absorbance, were dialyzed, in 500 times the volume of the fractions, against 50 mM HEPES pH 7.5, 10 mM CaCl₂, 0.05% Brij 35, overnight at 4° C. Dialysis was continued for an additional 24 hours in two fresh buffer changes. The dialyzed fractions were analyzed on SDS-PAGE and pooled based on purity. The pooled protein was concentrated to 1.2 mg/mL in Jumbo Sep centrifugal concentrators with 10,000 MWCO membranes. Protein concentration was determined with DC™ protein assay (Bio-Rad Laboratories, Inc.). The protein was aliquoted, frozen in liquid nitrogen and stored at −80° C.

Full-Length Rat proMMP9

Amino acid numbering for full-length rat proMMP9 was based on UniProtKB/Swiss-Prot P50282, full-length rat matrix metalloproteinase-9 precursor, proMMP9(1-708) (SEQ ID NO:11). The full-length rat proMMP9 was produced with the same methods as described for full-length human proMMP9. In brief, full-length rat proMMP9(1-708) (SEQ ID NO:11) was expressed in HEK293 cells as a secreted protein using a pcDNA3.1 expression vector. When expressed in HEK293 cells and secreted into the media, there is cotranslational removal of the signal peptide, so the final purified full-length rat proMMP9(1-708) (SEQ ID NO:11) protein lacks the signal peptide.

Human proMMP13

The sequence for proMMP13 was amino acids 1-268 from UniProtKB/Swiss-Prot P45452, proMMP13(1-268) (SEQ ID NO:7). The expression construct included a C-terminal Tev cleavage sequence flanking recombination sequences for use in the Invitrogen Gateway system. The construct was recombined into an entry vector using the Invitrogen Gateway recombination reagents. The resulting construct was transferred into a HEK293 expression vector containing a C-terminal 6×-histidine tag. Protein was expressed via transient transfection utilizing HEK293 cells and secreted into the media. When expressed in HEK293 cells and secreted into the media, there is cotranslational removal of the signal peptide, amino acids 1-19 of proMMP13(1-268) (SEQ ID NO:7). The final purified proMMP13(1-268) (SEQ ID NO:7) protein lacks the signal peptide. HEK293 media were harvested and centrifuged. Media were loaded on GE Healthcare HisTrap FF columns, washed with buffer A (20 mM Tris pH 7.5, 200 mM NaCl, 2 mM CaCl₂, 10 mM imidazole), and eluted with buffer B (20 mM Tris pH 7.5, 200 mM NaCl, 2 mM CaCl₂, 200 mM imidazole). The eluted protein was loaded on a Superdex 200 column equilibrated with buffer C (20 mM HEPES pH 7.4, 100 mM NaCl, 0.5 mM CaCl₂). Fractions containing proMMP13(1-268) (SEQ ID NO:7) were pooled and concentrated to >2 mg/mL.

### Human Catalytic MMP3

Catalytic MMP3 was amino acids 100-265 of human MMP3 from UniProtKB/Swiss-Prot P08254, MMP3(100-265) (SEQ ID NO:8). The corresponding nucleotide sequence was subcloned into a pET28b vector to add a C-terminal 6×-Histidine tag and the construct was used for expression in E. coli. The protein was purified to >95% purity from 4.5 M urea solubilized inclusion bodies by standard techniques. Aliquots of purified protein were stored at −70° C. Purified recombinant human catalytic MMP3 is also available from commercial sources (e.g., Calbiochem®, 444217).

### Biological Assays

### Catalytic Enzyme Assays

Selected compounds that were active in the proMMP9 activation assays were subsequently tested in catalytic MMP3 and catalytic MMP9 assays. Compounds that inhibited catalytic MMP3 or catalytic MMP9 were considered false positives in the proMMP9 activation assay.

### Catalytic MMP3

Compounds were assessed for inhibition of human catalytic MMP3, MMP3(100-265) (SEQ ID NO:8), using a peptide (Mca-RPKPVE-Nva-WRK(Dnp)-NH₂, Bachem M2110) that fluoresces upon cleavage by catalytic MMP3. The assay buffer employed was 50 mM Hepes, pH 7.5, 10 mM CaCl₂, 0.05% Brij-35. DMSO was included at a final concentration of 2%, arising from the test compound addition. The reaction volume was 100 μL. In 96-well black plates (Costar 3915), 44 μL of assay buffer was mixed with 1.0 μL of test compound, and 5 μL of 400 nM human catalytic MMP3 and the mixture was preincubated at 37° C. for 10 minutes. The reaction was initiated with 50 μL of 40 μM M-2110 substrate (freshly diluted in assay buffer), and the resulting activity associated with catalytic MMP3 was kinetically monitored at 328 nm excitation, 393 nm emission for 5-15 min at 37° C., using a Spectramax Gemini XPS reader (Molecular Devices). Initial velocities were plotted by use of a four-parameter logistics equation (GraphPad Prism® software) for determination of IC₅₀, if required. Final concentrations employed were 20 nM catalytic MMP3 and 20 μM M2110 substrate.

### Catalytic MMP9

Compounds were assessed for inhibition of human catalytic MMP9 (BioMol SE-244), using a peptide (Mca-PLGL-Dpa-AR-NH₂, BioMol P-126) that fluoresces upon cleavage by catalytic MMP9. The assay buffer employed was 50 mM Hepes, pH 7.5, 10 mM CaCl₂, 0.05% Brij-35. DMSO was included at a final concentration of 2%, arising from the test compound addition. The reaction volume was 100 μL. In 96-well black plates (Costar 3915), 44 μL of assay buffer was mixed with 1.0 μL of test compound, and 5 μL of 100 nM human catalytic MMP9 and the mixture was preincubated at 37° C. for 10 minutes. The reaction was initiated with 50 μL of 40 μM P-126 substrate (freshly diluted in assay buffer), and the resulting activity associated with catalytic MMP9 was kinetically monitored at 328 nm excitation, 393 nm emission for 5-15 min at 37° C., using a Spectramax Gemini XPS reader (Molecular Devices). Initial velocities were plotted by use of a four-parameter logistics equation (GraphPad Prism® software) for determination of IC₅₀, if required. Final concentrations employed were 5 nM catalytic MMP9 and 20 μM P-126 substrate.

The following examples were tested for inhibition of catalytic MMP9 and MMP3.

TABLE 1

| | Catalytic MMP9 | Catalytic MMP3 |
|---|---|---|
| Example | IC50 (μM) | IC50 (μM) |
| 4 | >50 | >50 |
| 59 | >50 | >50 |
| 65 | ~40 | >50 |
| 67 | >50 | >50 |

### ThermoFluor® Assays

### Generalized ThermoFluor® Methods

The ThermoFluor® (TF) assay is a 384-well plate-based binding assay that measures thermal stability of proteins (Biomol Screen 2001, 6, 429-40; Biochemistry 2005, 44, 5258-66). The experiments were carried out using instruments available from Johnson & Johnson Pharmaceutical Research & Development, LLC. TF dye used in all experiments was 1,8-anilinonaphthalene-8-sulfonic acid (1,8-ANS) (Invitrogen: A-47).

Compounds were arranged in a pre-dispensed plate (Greiner Bio-one: 781280), wherein compounds were serially diluted in 100% DMSO across 11 columns within a series. Columns 12 and 24 were used as DMSO reference and contained no compound. For multiple compound concentration-response experiments, the compound aliquots (50 mL) were robotically predispensed directly into black 384-well polypropylene PCR microplates (Abgene: TF-0384/k) using a Cartesian Hummingbird liquid handler (DigiLab, Holliston, Mass.). Following compound dispense, protein and dye solutions were added to achieve the final assay volume of 3 μL. The assay solutions were overlayed with 1 μL of silicone oil (Fluka, type DC 200: 85411) to prevent evaporation.

Assay plates were robotically loaded onto a thermostatically controlled PCR-type thermal block and then heated from 40 to 90° C. at a ramp-rate of 1° C./min for all experiments. Fluorescence was measured by continuous illumination with UV light (Hamamatsu LC6) supplied via fiber optics and filtered through a band-pass filter (380-400 nm; >6 OD cutoff). Fluorescence emission of the entire 384-well plate was detected by measuring light intensity using a CCD camera (Sensys, Roper Scientific) filtered to detect 500±25 nm, resulting in simultaneous and independent readings of all 384 wells. A single image with 20-sec exposure time was collected at each temperature, and the sum of the pixel intensity in a given area of the assay plate was recorded vs temperature and fit to standard equations to yield the Tₘ (J Biomol Screen 2001, 6, 429-40).

Thermodynamic parameters necessary for fitting compound binding for each proMMP were estimated by differential scanning calorimetry (DSC) and from ThermoFluor® data. The heat capacity of unfolding for each protein was estimated from the molecular weight and from ThermoFluor® dosing data. Unfolding curves were fit singly, then in groups of 12 ligand concentrations the data were fit to a single K_{D} for each compound.

ThermoFluor® with proMMP9(67-444;ΔFnII) (SEQ ID NO:5)

The protein sample preparations had to include a desalting buffer exchange step via a PD-10 gravity column (GE Healthcare). The desalting buffer exchange was performed prior to diluting the protein to the final assay concentration of 3.5 μM proMMP9(67-444;ΔFnII) (SEQ ID NO:5). The concentration of proMMP9(67-444;ΔFnII) (SEQ ID NO:5) was determined spectrophotometrically based on a calculated extinction coefficient of ε₂₈₀=33900 M⁻¹ cm⁻¹, a calculated molecular weight of 22.6 kDa, and calculated pI of 5.20. ThermoFluor® reference conditions were defined as follows: 80 μg/mL (3.5 μM) proMMP9(67-444;ΔFnII) (SEQ ID NO:5), 50 μM 1,8-ANS, pH 7.0 Buffer (50 mM HEPES pH 7.0, 100 mM NaCl, 0.001% Tween-20, 2.5 mM MgCl₂, 300 μM CaCl₂). The thermodynamic parameters for proMMP9(67-444;ΔFnII) (SEQ ID NO:5) are as follows: Tₘ (° C.)=63 (+/−0.1), Δ_{U}H_{(Tm)} (cal mol⁻¹)=105000(+/−5000), Δ_{U}S_{(Tm)} (cal mol⁻¹ K⁻¹)=450, Δ_{U}Cₚ (cal mol⁻¹ K⁻¹)=2000.

ThermoFluor® with proMMP9(20-445;ΔFnII) (SEQ ID NO:6)

The protein sample preparations included a desalting buffer exchange step via a PD-10 gravity column (GE Healthcare). The desalting buffer exchange was performed prior to diluting the protein to the final assay concentration of 2.8 μM proMMP9(20-445;ΔFnII) (SEQ ID NO:6). The concentration of proMMP9(20-445;ΔFnII) (SEQ ID NO:6) was determined spectrophotometrically based on a calculated extinction coefficient of ε₂₈₀=39880 M⁻¹ cm⁻¹, a calculated molecular weight of 28.2 kDa, and calculated pI of 5.5. ThermoFluor® reference conditions were defined as follows: 80 μg/mL (2.8 μM) proMMP9(20-445;ΔFnII) (SEQ ID NO:6), 50 μM 1,8-ANS, pH 7.0 Buffer (50 mM HEPES pH 7.0, 100 mM NaCl, 0.001% Tween-20, 2.5 mM MgCl₂, 300 μM CaCl₂). The thermodynamic parameters for proMMP9(20-445;ΔFnII) (SEQ ID NO:6) are as follows: Tₘ (° C.)=72 (+/−0.1), Δ_{U}H_{(Tm)} (cal mol⁻¹)=160000(+/−5000), Δ_{U}S_{(Tm)} (cal mol⁻¹ K⁻¹)=434, Δ_{U}Cₚ (cal mol⁻¹ K⁻¹)=2400.

ThermoFluor® with proMMP13(1-268) (SEQ ID NO: 7)

The proMMP13(1-268) (SEQ ID NO:7) protein sample preparations included a desalting buffer exchange step via a PD-10 gravity column (GE Healthcare). The desalting buffer exchange was performed prior to diluting the protein to the final assay concentration of 3.5 μM. The concentration of proMMP13(1-268) (SEQ ID NO:7) was estimated spectrophotometrically based on a calculated extinction coefficient of ε₂₈₀=37000 M⁻¹ cm⁻¹, a calculated molecular weight of 30.8 kDa, and calculated pI of 5.33. ThermoFluor® reference conditions were defined as follows: 100 μg/mL proMMP13(1-268) (SEQ ID NO:7), 25 μM 1,8-ANS, pH 7.0 Buffer (50 mM HEPES pH 7.0, 100 mM NaCl, 0.001% Tween-20, 2.5 mM MgCl₂, 300 μM CaCl₂). The thermodynamic parameters for proMMP13(1-268) (SEQ ID NO:7) are as follows: Tₘ (° C.)=67 (+/−0.1), Δ_{U}H_{(Tm)} (cal mol⁻¹)=107000(+/−5000), Δ_{U}S_{(Tm)} (cal mol⁻¹ K⁻¹)=318, Δ_{U}Cₚ (cal mol⁻¹ K⁻¹)=2600.

ThermoFluor® data for example compounds is shown in Table 2.

TABLE 2

| | proMMP9 | proMMP9 | proMMP13 |
|---|---|---|---|
| | (20-445; ΔFnII) | (67-444; ΔFnII) | (1-268) |
| | (SEQ ID NO: 6) | (SEQ ID NO: 5) | (SEQ ID NO: 7) |
| Example | binding, Kd (μM) | binding, Kd (μM) | binding, Kd (μM) |
| 1 | 8.68 | 1.29 | 14.07 |
| 2 | >95 | 55.32 | 42.49 |
| 3 | 3.81 | 1.15 | ND |
| 4 | 1.70 | 0.254 | ND |
| 5 | >95 | 5.49 | 20.04 |
| 6 | 0.907 | 0.156 | 15.07 |
| 7 | 12.91 | 8.84 | 44.55 |
| 8 | 10.75 | 30.16 | 26.85 |
| 9 | 19.03 | >81 | ND |
| 10 | 9.61 | 3.67 | 55.88 |
| 11 | >95 | 19.94 | 58.95 |
| 12 | 13.85 | 2.92 | 12.01 |
| 13 | >95 | >95 | >95 |
| 14 | >95 | ND | 9.12 |
| 15 | >95 | 43.34 | ND |
| 16 | >95 | 8.90 | >95 |
| 17 | 15.61 | 3.99 | 10.68 |
| 18 | 2.89 | 0.376 | ND |
| 19 | >52 | 3.86 | ND |
| 20 | 2.15 | 0.414 | ND |
| 21 | 4.48 | 0.140 | 3.57 |
| 22 | 12.34 | 2.32 | 44.25 |
| 23 | >35 | 5.53 | ND |
| 24 | 0.370 | >38 | ND |
| 25 | 12.50 | 16.33 | ND |
| 26 | >95 | >95 | 4.78 |
| 27 | 49.94 | ND | ND |
| 28 | >65 | 22.62 | >95 |
| 29 | ND | ND | ND |
| 30 | >95 | 13.83 | 42.01 |
| 31 | 3.34 | 0.645 | ND |
| 32 | >9.2 | 9.06 | ND |
| 33 | 3.75 | 0.695 | ND |
| 34 | 3.64 | 0.820 | 43.50 |
| 35 | 10.28 | 3.92 | ND |
| 36 | >11 | >11 | ND |
| 37 | 5.16 | 4.48 | >95 |
| 38 | >8.3 | >8.3 | ND |
| 39 | 6.35 | 2.49 | ND |
| 40 | >18 | 16.31 | ND |
| 41 | 1.94 | ND | ND |
| 42 | 28.82 | ND | 5.35 |
| 43 | 76.67 | 13.10 | 20.35 |
| 44 | >95 | 44.66 | >95 |
| 45 | 1.65 | 10.05 | >95 |
| 46 | 65.22 | 36.71 | 16.04 |
| 47 | >90 | 40.95 | >95 |
| 48 | 17.50 | 4.73 | ND |
| 49 | >17 | 8.70 | ND |
| 50 | >14 | 4.05 | ND |
| 51 | >16 | >15 | ND |
| 52 | 7.27 | 9.09 | 13.17 |
| 53 | 28.62 | 15.08 | >95 |
| 54 | >65 | 17.90 | ND |
| 55 | >95 | 22.51 | >95 |
| 56 | >70 | 14.64 | ND |
| 57 | 21.82 | 23.68 | ND |
| 58 | 13.36 | 3.01 | >95 |
| 59 | 29.21 | 0.583 | ND |
| 60 | 16.83 | 11.28 | 44.39 |
| 61 | 5.07 | 1.06 | 21.03 |
| 62 | 8.29 | 2.46 | 47.19 |
| 63 | 19.63 | 5.13 | ND |
| 64 | 47.49 | 45.34 | >95 |
| 65 | 1.75 | 0.388 | 37.48 |
| 66 | >6.1 | >6.1 | ND |
| 67 | 2.43 | 0.318 | ND |
| 68 | 3.76 | 1.12 | 14.52 |
| 69 | >15 | >15 | ND |
| 70 | 5.79 | 11.96 | 0.668 |
| 71 | 11.71 | 6.56 | ND |
| 72 | 6.53 | >58 | ND |
| 73 | 25.51 | 15.91 | 18.00 |
| 74 | 7.89 | 66.14 | >95 |
| 75 | 17.87 | 10.09 | 28.93 |
| 76 | 26.95 | 10.80 | 57.09 |
| 77 | >77 | 20.57 | ND |
| 78 | 0.879 | 0.146 | ND |
| 79 | 21.44 | 3.93 | ND |

### Enzyme Assays

proMMP9/MMP3 P126 Activation Assay

Compounds were assessed for inhibition of proMMP9 activation by catalytic MMP3, MMP3(100-265) (SEQ ID NO:8) using full-length proMMP9(1-707) (SEQ ID NO:1) purified from HEK293 cells and a peptide (Mca-PLGL-Dpa-AR-NH₂, BioMol P-126) that fluoresces upon cleavage by catalytic MMP9. The assay buffer employed was 50 mM Hepes, pH 7.5, 10 mM CaCl₂, 0.05% Brij-35. DMSO was included at a final concentration of 2%, arising from the test compound addition. On the day of assay, proMMP9(1-707) (SEQ ID NO:1) purified from HEK293 cells and MMP3(100-265) (SEQ ID NO:8) were diluted to 400 nM in assay buffer. The reaction volume was 50 μL. In 96-well black plates (Costar 3915), 44 μL of assay buffer was mixed with 1.0 μL of test compound, 2.5 μL of 400 nM proMMP9(1-707) (SEQ ID NO:1) purified from HEK293 cells and the reaction was initiated with 2.5 μL of 400 nM MMP3(100-265) (SEQ ID NO:8).The plate was sealed and incubated for 80 min at 37° C. Final concentrations were 20 nM proMMP9(1-707) (SEQ ID NO:1) purified from HEK293 cells and 20 nM MMP3(100-265) (SEQ ID NO:8), and concentrations of test compounds were varied to fully bracket the IC₅₀. Immediately following the 80 min incubation, 50 μL of 40 μM P-126 substrate was added (freshly diluted in assay buffer), and the resulting activity associated with catalytic MMP9 was kinetically monitored at 328 nm excitation, 393 nm emission for 10-15 min at 37° C., using a Spectramax Gemini XPS reader (Molecular Devices). Reactivity of residual MMP3 towards P-126 substrate was minimal under these conditions. Initial velocities were plotted by use of a four-parameter logistics equation (GraphPad Prism® software) for determination of IC₅₀.

### ProMMP9/MMP3 DQ Gelatin Activation Assay

Compounds were assessed for inhibition of proMMP9 activation by catalytic MMP3 using a quenched fluorescein gelatin substrate (DQ gelatin, Invitrogen D12054) that fluoresces upon cleavage by activated MMP9. The assay buffer employed was 50 mM Hepes, pH 7.5, 10 mM CaCl₂, 0.05% Brij-35. DMSO was included at a final concentration of 0.2%, arising from the test compound addition. On the day of assay, full-length proMMP9(1-707) (SEQ ID NO:1) from COS-1 cells and catalytic MMP3(100-265) (SEQ ID NO:8) were diluted to 60 nM and 30 nM, respectively, in assay buffer. Test compounds in DMSO were diluted 250-fold in assay buffer at 4× the final concentration. The reaction volume was 12 μL, and all reactions were conducted in triplicate. In 384-well half-volume plates (Perkin Elmer ProxiPlate 384 F Plus, 6008260), 4 μL of test compound in assay buffer was mixed with 4 μL of 60 nM full-length proMMP9(1-707) (SEQ ID NO:1) from COS-1 cells. The plate was sealed and incubated for 30 min at 37° C. Final concentrations were 20 nM full-length proMMP9(1-707) (SEQ ID NO:1) from COS-1 cells and 10 nM MMP3(100-265) (SEQ ID NO:8), and concentrations of test compounds were varied to fully bracket the IC₅₀. Immediately following the 30 min incubation, 4 μL of 40 μg/ml DQ gelatin substrate was added (freshly diluted in assay buffer), and incubated for 10 min at room temperature. The reaction was stopped by the addition of 4 μL of 50 mM EDTA, and the resulting activity associated with catalytic MMP9 was determined at 485 nm excitation, 535 nm emission using an Envision fluorescent reader (Perkin Elmer). Reactivity of residual MMP3 towards DQ gelatin was minimal under these conditions. Percent inhibition of test compounds were determined from suitable positive (DMSO only in assay buffer) and negative (EDTA added prior to reaction initiation) controls. Plots of % inhibition vs. test compound concentration were fit to a four-parameter logistics equation (GraphPad Prism® software) for determination of IC₅₀.

Enzyme assay data for example compounds is shown in Table 3.

TABLE 3

| | proMMP9/MMP3 P126 | ProMMP9/MMP3 |
|---|---|---|
| | Activation Assay, IC₅₀ | DQ gel, IC₅₀ |
| Example | (μM) | (μM) |
| 1 | 2.5 | ND |
| 2 | 2.5 | ND |
| 3 | 2.0 | ND |
| 4 | 1.5 | ND |
| 5 | 0.71 | ND |
| 6 | 0.65 | ND |
| 7 | ND | ND |
| 8 | ND | ND |
| 9 | ND | ND |
| 10 | ND | ND |
| 11 | ND | ND |
| 12 | 3.6 | ND |
| 13 | ~2 | ND |
| 14 | 3.6 | ND |
| 15 | 2.5 | ND |
| 16 | 3.6 | ND |
| 17 | ND | ND |
| 18 | 2.6 | ND |
| 19 | ND | ND |
| 20 | 3.3 | ND |
| 21 | 2.8 | ND |
| 22 | ND | ND |
| 23 | ND | 26.2 |
| 24 | ND | >20 |
| 25 | ND | >20 |
| 26 | ND | 13.8 |
| 27 | 3.1 | ND |
| 28 | ND | 15.6 |
| 29 | ND | 14.4 |
| 30 | ND | ND |
| 31 | ND | 3.3 |
| 32 | ND | >20 |
| 33 | ND | 2.9 |
| 34 | 4.0 | 2.3 |
| 35 | ND | 7.0 |
| 36 | ND | 1.7 |
| 37 | ND | >20 |
| 38 | ND | 9.6 |
| 39 | ND | 17.1 |
| 40 | ND | >20 |
| 41 | 1.9 | ND |
| 42 | 5.0 | ND |
| 43 | ND | >20 |
| 44 | ND | 11.9 |
| 45 | >50 | >20 |
| 46 | ND | 8.6 |
| 47 | ND | 5.2 |
| 48 | ND | ND |
| 49 | ND | 5.7 |
| 50 | ND | 21.2 |
| 51 | ND | 10.1 |
| 52 | ND | 3.3 |
| 53 | ND | 4.7 |
| 54 | ND | 16.1 |
| 55 | ND | 13.0 |
| 56 | ND | >20 |
| 57 | ND | 7.9 |
| 58 | ND | 2.6 |
| 59 | 0.52 | 0.34 |
| 60 | ND | 5.5 |
| 61 | ND | 1.6 |
| 62 | ND | 3.4 |
| 63 | ND | 28.2 |
| 64 | ND | 2.1 |
| 65 | 1.3 | 1.5 |
| 66 | ND | 1.9 |
| 67 | 2.1 | ND |
| 68 | 2.5 | 3.1 |
| 69 | ND | 11.2 |
| 70 | >20 | ND |
| 71 | ND | 6.9 |
| 72 | ND | 3.1 |
| 73 | ND | 17.9 |
| 74 | ND | 21.1 |
| 75 | ND | 17.9 |
| 76 | ND | 16.3 |
| 77 | 3.8 | ND |
| 78 | 0.13 | ND |
| 79 | 1.0 | ND |

### Cell-Based Assays

Activation of proMMP9 in Rat Synoviocyte Cultures

A primary synoviocytes line was derived from the periarticular tissue of arthritic rats. Arthritis was induced in female Lewis rats following an i.p. administration of streptococcal cell wall peptidoglycan polysaccharides (J Exp Med 1977; 146:1585-1602). Rats with established arthritis were sacrificed, and hind-limbs were severed, immersed briefly in 70% ethanol, and placed in a sterile hood. The skin was removed and the inflamed tissue surrounding the tibia-tarsal joint was harvested using a scalpel. Tissue from six rats was pooled, minced to approximately 8 mm³ pieces, and cultured in Dulbecco's Modified Eagle's Medium (DMEM) containing 15% fetal calf serum (FCS). In the following weeks, cells migrated out of the tissue piece, proliferated, and formed a monolayer of adherent cells. The synoviocytes were lifted from culture plates with 0.05% trypsin and passaged weekly at 1:4 ratios in DMEM containing 10% FCS. Synoviocytes were used at passage 9 to investigate the ability of Compound α to inhibit the maturation of MMP9 to active form.

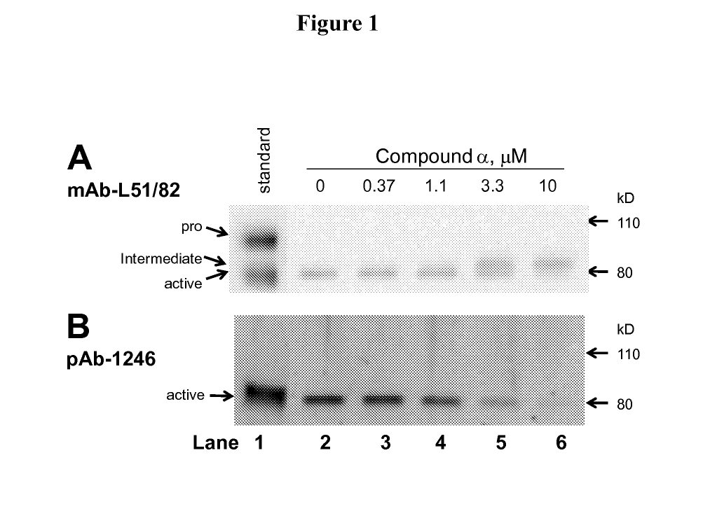

Rat synoviocytes spontaneously expressed and activated MMP9 when cultured in collagen gels and stimulated with tumor necrosis factor-alpha (TNFα) (FIG. 1 and Table 4). Eight volumes of an ice-cold solution of 3.8 mg/mL rat tail collagen (Sigma Cat #C3867-1VL) were mixed with 1 volume of 1 M sodium bicarbonate and 1 volume of 10× Roswell Park Memorial Institute medium. The pH of the mixture was adjusted to pH 7 with 1 N sodium hydroxide and equal volumes of the pH-adjusted collagen solution were mixed with DMEM containing 0.8 million synoviocytes per mL. One half mL volumes were dispensed into Costar 24-well culture dishes and placed for one hr at 37° C. and 5% CO₂, during which time the collagen solution formed a gel. Individual gels were dislodged into wells of 12-well Costar plates containing 1 mL/well of DMEM adjusted to contain 0.05% BSA and 100 ng/mL mouse TNFα (R&D Systems Cat #410-MT-010). The plates were agitated 10 seconds to ensure that the collagen gels did not adhere to the well bottoms. After overnight culture at 37° C. and 5% CO₂, wells were adjusted to contain an additional 0.5 mL of DMEM containing 0.05% BSA and Compound α at 4× the final desired concentration (final culture volumes were 2 mL). The plates were cultured an additional 48 hrs, at which time 1 mL of conditioned media were harvested into fresh eppendorf tubes containing 40 μL/mL of a 50% slurry of gelatin-conjugated sepharose (GE Healthcare Cat #17-0956-01). Samples were rotated for 2 hrs at 4° C. before centrifugation 1 min×200 g. Supernatants were discarded. The gelatin-sepharose pellets were washed once with 1 mL of ice cold DMEM, resuspended in 50 μL of 2× reducing Leamli buffer and heated 5 min at 95° C. Fifteen μL of eluted proteins were resolved on 4-12% NuPAGE gels and transferred to 0.45 nm pore-sized nitrocellose blots. Next, blots were incubated in blocking buffer (5% milk in Tris-buffered saline containing 0.1% Tween-20) for 1 hr at RT and probed overnight (4° C.) with blocking buffer containing 1 μg/mL primary antibodies. Blots were next probed 1 hr at RT with 1/10,000 dilutions of goat anti-mouse IgG-HRP or goat anti-rabbit IgG-HRP (Santa Cruz) in blocking buffer and developed using SuperSignal® West Fempto Maximum Sensitivity Substrate. Chemiluminesence signal was analyzed using a ChemiDoc imaging system (BioRad Laboratories) and Quantity One® image software. Electrophoretic mobility was estimated based on the mobility of standards (Novex Sharp Pre-Stained Protein Standards P/N 57318). Mouse mAb-L51/82 (UC Davis/NIH NeuroMab Facility, Antibody Incorporated) was used to detect pro and processed forms of MMP9. Synoviocyte-conditioned media contained an approximately 80 kD form of MMP9 (FIG. 1A, lane 2). In the presence of 0.37-10 μM Compound α (FIG. 1A, lanes 3-6), the 80 kD active MMP9 form was reduced in a dose dependent fashion, and a form of approximately 86 kD appeared. The 86 kD form was predominant in the presence of 10 μM Compound α (FIG. 1A, lane 6). Lane 1 was loaded with a standard containing 3 ng of full-length rat proMMP9(1-708) (SEQ ID NO:11) and 3 ng of full-length rat proMMP9(1-708) (SEQ ID NO:11) converted to catalytic rat MMP9 by catalytic MMP3. The electrophoretic mobility of the 80 kD form present in synoviocyte conditioned medium was the same as the active MMP9 standard. The 86 kD form produced by synoviocytes in the presence of Compound α demonstrated greater mobility than the full-length rat proMMP9(1-708) (SEQ ID NO:11) standard which ran with a mobility of approximately 100 kD. The 86 kD form demonstrated a mobility similar to an incompletely processed intermediate form described previously that retains the cysteine switch and lacks catalytic activity (J Biol Chem; 1992; 267:3581-4).

ProMMP9 is activated when cleaved between R106 and F107 (J Biol Chem; 1992; 267:3581-4). A rabbit polyclonal antibody (pAb-1246) was generated to the active MMP9 N-terminal neoepitope using an approach similar to that reported previously (Eur J Biochem; 1998; 258:37-43). Rabbits were immunized and boosted with a peptide, human MMP9(107-113) (SEQ ID NO:9) conjugated to keyhole limpet hemocyanin, and antibodies were affinity purified from serum using FQTFEGD-conjugated agarose affinity resin and 100 mM glycine (pH 2.5) elution. To resolve N-terminal neoepitope antibodies from antibodies directed to other epitopes within the sequence, eluted antibody was dialyzed in PBS and cross-absorbed by mixing with a peptide, human proMMP9(99-113) (SEQ ID NO:10), that was conjugated to agarose. The unbound fraction containing N-terminal neoepitope antibodies was recovered and was designated pAb-1246.

FIG. 1B, lane 1 demonstrated that pAb-1246 bound the 80 kD active MMP9 standard, but did not recognize the 100 kD proMMP9 standard. pAb-1246 detected 80 kD active MMP9 in synoviocyte conditioned medium, and Compound α caused a dose-dependent reduction in active MMP9 (FIG. 1B, lanes 2-6). Band chemiluminescence intensities were measured directly and reported in Table 4. The production of active MMP9 was inhibited by Compound α with an IC₅₀ of approximately 1.1 μM. pAb-1246 did not recognize the 86 kD form, providing further evidence that this likely represented an intermediate form whose further maturation was blocked by Compound α.

TABLE 4
Compound α blocked production
of active MMP9 by rat synoviocytes ^{a}

| | Signal of 80 kD band | |
|---|---|---|
| Compound α, μM | (INT*mm²) ^{b} | % Inhibition ^{c} |
| 0 | 84384 | 0 |
| 0.37 μM | 74381 | 12 |
| 1.1 μM | 45381 | 46 |
| 3.3 μM | 11554 | 86 |
| 10 μM | 2578 | 97 |

^{a} Rat synoviocytes embedded in collagen gels were stimulated 72 hrs with TNFα. Cultures were supplemented with the indicated concentrations of Compound α for the final 48 hrs and conditioned media were assessed for the 80 kD active form of MMP9 by Western blotting with pAb-1246 developed against the N-terminal activation neoepitope.
^{b} Chemiluminesence captured during a 30 s exposure was analyzed using a ChemiDoc imaging system (BioRad Laboratories) and Quantity One ® image software. Signals were measured within uniform sized boxes drawn to circumscribe the 80 kD bands and were the product of the average intensity (INT) and the box area (mm²). Values given have been corrected for background signal.
^{c} Percent signal reduction relative to the signal generated by synoviocytes cultured in the absence of Compound α.

Activation of proMMP9 by Human Fetal Lung Fibroblast Cultures

Compound α was assessed additionally for ability to block the maturation of proMMP9 to active MMP9 in cultures of human fetal lung fibroblasts (HFL-1, American Type Culture Collection #CCL-153). Unlike rat synoviocytes, HFL-1 cells were unable to process proMMP9 to the active form without addition of neutrophil elastase. Elastase did not directly cause processing of recombinant proMMP9 (data not shown). Rather, the function of elastase in this assay may be to inactivate tissue inhibitors of matrix metalloproteinases (TIMPs) that repress endogenous pathways of MMP9 activation (Am J Respir Crit Care Med; 1999; 159:1138-46).

HLF-1 were maintained in monolayer culture in DMEM with 10% FCS and were used between passage numbers 5-15. HLF-1 were embedded in collagen gels as described for rat SCW synoviocytes (vida supra). Half mL gels containing 0.4 million cells were dislodged into wells of 12 well Costar plates containing 1 mL/well of DMEM adjusted to contain 0.05% BSA and 100 ng/mL human TNFα (R&D Systems Cat #210-TA/CF). After overnight culture (37° C. and 5% CO₂) wells were adjusted to contain an additional 0.5 mL of DMEM containing 0.05% BSA and with or without 13.2 μM Compound α (final concentration was 3.3 μM Compound-α). Next, cultures were adjusted to contain 30 nM human elastase (Innovative Research). The plates were cultured an additional 72 hrs, at which time MMP9 secreted into the conditioned media was bound to gelatin-sepharose and evaluated by Western blot analysis as described for the rat synoviocyte cultures (vida supra). mAb-51/82 detected three forms of MMP9 in HFL-1 cultures.

These included a form of approximately 100 kD with mobility similar to recombinant rat proMMP9, an approximately 80 kD form with mobility similar to rat active MMP9, and an approximately 86 kD intermediate form. The band intensities are provided in Table 5. In the absence of Compound α, most of the MMP9 was present as the 80 kD form. In the presence of Compound α, the 80 kD form was a minor fraction of the total signal while nearly half of the signal were contributed each by the 100 kD and 86 kD forms. The total signal of the three bands was similar with or without Compound α. These data indicate that the 100 kD and 86 kD forms of MMP9 were effectively stabilized by Compound α and the formation of the 80 kD form was suppressed.

TABLE 5
Compound α blocked processing of MMP9 by HFL-1 cells ^{a}

| Com- | Signal (INT*mm²) ^{b} | | | | Percent of total signal | | |
|---|---|---|---|---|---|---|---|
| pound α, | 100 | 86 | 80 | | 100 | 86 | 80 |
| 3.3 μM | kD | kD | kD | Total | kD | kD | kD |
| − | 17190 | 24858 | 61925 | 103973 | 16 | 24 | 60 |
| + | 42107 | 43147 | 6092 | 91346 | 46 | 47 | 7 |

^{a} Human fetal lung fibroblasts (HFL-1) embedded in collagen gels were stimulated 90 hrs with TNFα. Cultures were supplemented with or without 3.3 μM Compound α and with 30 nM elastase for the final 72 hrs and conditioned media were assessed for the MMP9 forms by Western blotting with mAb-L51/82.
^{b} Chemiluminesence captured during a 150 s exposure was analyzed using a ChemiDoc imaging system (BioRad Laboratories) and Quantity One ® image software. Signals were measured within uniform sized boxes drawn to circumscribe the bands and were the product of the average intensity (INT) and the box area (mm²). Values given have been corrected for background signal.

A second experiment was performed to determine if the 80 kD form was mature active MMP9 and to determine the potency of Compound α as an inhibitor of MMP9 maturation in this assay. HFL-1 cells embedded in collagen gels were cultured as described above in the presence of TNFα overnight and the cultures were then adjusted to contain 30 nM elastase and graded concentrations of Compound α for an additional 72 hrs at which time MMP9 secreted into the conditioned media was bound to gelatin-sepharose and evaluated by Western blot analysis for active MMP9 using pAb-1246 raised against the N-terminal neoepitope of active MMP9 (Table 6). In the absence of Compound α, pAb-1246 readily detected MMP9 with an electrophoretic mobility of approximately 80 kD. Compound α effectively inhibited the ability of HFL-1 cultures to process proMMP9 to active MMP9. Inhibition occurred over a dose range with an IC₅₀ of approximately 0.3 μM Compound α.

TABLE 6
Compound α blocked production of active
MMP9 by human fetal lung fibroblasts ^{a}

| | Signal of 80 kD band | |
|---|---|---|
| Compound α, μM | (INT*mm²) ^{b} | % Inhibition ^{c} |
| 0 | 168781 | 0 |
| 0.12 μM | 168211 | 0 |
| 0.37 μM | 45996 | 73 |
| 1.1 μM | 1747 | 99 |
| 3.3 μM | 152 | 100 |
| 10 μM | 0 | 100 |

^{a} Human fetal lung fibroblasts (HFL-1) embedded in collagen gels were stimulated 90 hrs with TNFα. Cultures were supplemented with the indicated concentrations of Compound α and 30 nM elastase for the final 72 hrs and conditioned media were assessed for active MMP9 by Western blotting with pAb-1246 developed against the N-terminal activation neoepitope.
^{b} Chemiluminesence captured during a 10 s exposure was analyzed using a ChemiDoc imaging system (BioRad Laboratories) and Quantity One ® image software. Signals were measured within uniform sized boxes drawn to circumscribe the 80 kD bands and were the product of the average intensity (INT) and the box area (mm²). Values given have been corrected for background signal.
^{c} Percent signal reduction relative to the signal generated by HFL-1 cells cultured in the absence of Compound α.

### In Vivo Studies

Expression and Activation of proMMP9 In Vivo is Associated with Rat SCW-Arthritis

MMP9 protein expression was reportedly increased in the synovial fluid of patients with rheumatoid arthritis (Clinical Immunology and Immunopathology; 1996; 78:161-71). A preliminary study was performed to assess MMP9 expression and activation in a rat model of arthritis.

A polyarthritis can be induced in female Lewis rats following i.p. administration of streptococcal cell wall (SCW) proteoglycan-polysaccharides (PG-PS) (J Exp Med 1977; 146:1585-1602). The model has an acute phase (days 3-7) that is complement and neutrophil-dependent and that resolves. A chronic erosive phase begins at about day ten and is dependent on the development of specific T cell immunity to the PG-GS, which resists digestion and remains present in synovial macrophages for months. Like rheumatoid arthritis, SCW-induced arthritis is reduced by TNF inhibitors, and the dependence of SCW-induced arthritis on macrophages (Rheumatology; 2001; 40:978-987) and the strong association of rheumatoid arthritis severity with synovial-tissue macrophage counts (Ann Rheum Dis; 2005; 64:834-838) makes SCW-arthritis an attractive model for testing potential therapeutic agents.

SCW PG-PS 10S (Beckton Dickinson Cat#210866) suspended in saline was vortexed for 30 seconds and sonicated for 3 min with a probe type sonicator prior to injection. Female Lewis (LEW/N) rats, 5-6 weeks of age (80-100 g) were injected (i.p.) with SCW PG-PS (15 μg of rhamnose/gram BW) in the lower left quadrant of the abdomen using a 1 mL syringe fitted with a 23-gauge needle. Control (disease-free) rats were treated in a similar manner with sterile saline. Control rats were sacrificed on day 5 and groups of SCW-injected rats were sacrificed on day 5 when acute inflammation was maximal or on day 18 when chronic inflammation was established.

Hind-limbs were skinned, severed just above the tibia-tarsus joint and below the metatarsals, and the tibia-tarsus joints (ankles) were weighed, snap frozen and pulverized on dry ice using a hammer and anvil. The pulverized tissue was suspended in 3 volumes (w:v) of ice-cold homogenization buffer containing 50 mM Tris pH 7.5, 150 mM NaCl, 5 mM EDTA, 1% Triton×100, 0.05% Brij 30, 10% dimethylsulfoxide and Complete EDTA-free Protease Inhibitor Cocktail (Roche Diagnostics). The suspended tissue was homogenized sequentially with a Kinematica AG Polytron and a Dounce homogenizer. Homogenates were centrifuged at 16,000×g for 10 min at 4° C. and the soluble fractions were saved. Dimethylsulfoxide was removed from a portion of each soluble fraction using PD MiniTrap™ G-25 desalting columns (GE Healthcare). Homogenates (0.25 mL), free of DMSO, were diluted with an equal volume of binding buffer (i.e., homogenization buffer without dimethylsufoxide) and adjusted to contain 50 μL of a 50% slurry of gelatin-conjugated sepharose. Following 2 hours of rotation at 4° C. the beads were washed twice in binding buffer and eluted in 100 μL 2×-reducing Laemmli buffer with heating to 95° C. for 5 minutes. Eluates (20 μL) were resolved on 4-12% NuPAGE gels, transferred to 0.45 um pore-sized nitrocellose and immunoblotted for detection of proMMP9, active MMP9, and other processed forms using mAb-L51/82 and pAb-1246 as described above for detection of MMP9 forms in synoviocyte and HFL-1 cell conditioned media.

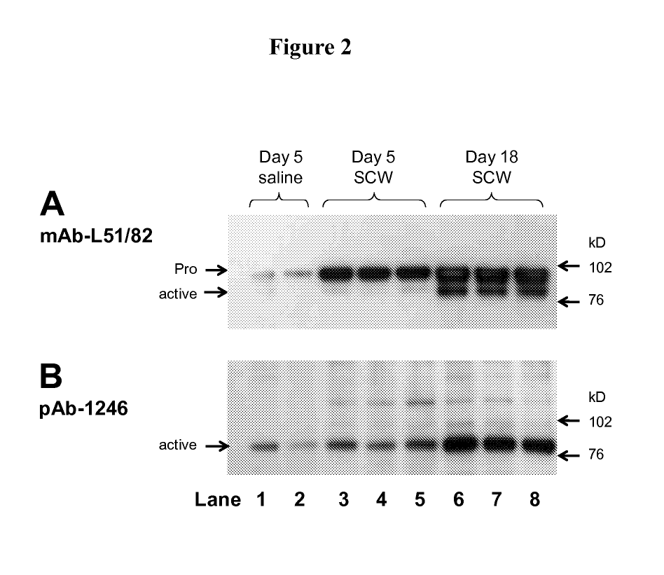

In healthy ankles of rats administered saline, mAb-L51/82 detected small amounts of an approximately 100 kD (proMMP9) and an approximately 80 kD form of MMP9 (FIG. 2A, lanes 1 and 2). proMMP9 was increased markedly in ankle homogenates 5 and 18 days after SCW-administration (FIG. 2A, lanes 3-5 and 6-8, respectively). The 80 kD MMP9 was increased mildly 5 days after SCW-administration (FIG. 2A, lanes 3-5) and was increased markedly 18 days after SCW-administration (FIG. 2A, lanes 6-8). In healthy ankles of rats administered saline, mAb-1246 detected small amounts active MMP9 at 80 kD (FIG. 2B, lanes 1 and 2). The 80 kD active MMP9 was increased mildly 5 days after SCW-administration (FIG. 2A, lanes 3-5) and was increased markedly 18 days after SCW-administration (FIG. 2A, lanes 6-8).

Efficacy of Compound α in Rats with SCW Arthritis

Having shown that active MMP9 is increased in rats with SCW-induced arthritis, we next sought to determine the ability of Compound α to reduce disease severity and to reduce active MMP9.

Compound α Reduced Ankle Swelling of Rats with SCW-Induced Arthritis

To induce arthritis, Female Lewis (LEW/N) rats, 5-6 weeks of age (80-100 g) were injected (i.p.) with SCW PG-PS as described above. Eighteen days later, arthritis was well established. Calipers were used to measure the width (anterior to posterior surface) of the left and right hind ankles of each rat. Each ankle was measured 3 times and averaged, and treatment groups were randomized based on ankle thickness (Table 7). Commencing on day 18, randomized groups of arthritic rats (n=5 rats/group) received vehicle or 5, 20, or 50 mg/kg Compound α BID by oral gavage. Vehicle consisted of an aqueous mixture containing 2% (v:v) N-methylpyrrolidone, 5% (v:v) glycerine, and 20% (w:v) captisol. Treatment continued daily through the morning of day 26.

By day 18 mean ankle thickness was increased an average of >4.4 mm compared to disease free rats. Rats treated with vehicle alone continued to gradually develop a more severe arthritis based on ankle thickness measurements over the eight-day treatment period (Table 7). Treatment with Compound α induced a dose-dependent decrease in ankle thickness measurements. By day 26, the disease associated increase in ankle thickness had been reduced 27, 37, and 46 percent by 5, 20, and 50 mg/kg Compound α, respectively.

TABLE 7
Ankle thickness of rats with SCW-arthritis
dosed with vehicle vs. Compound α

| | | Ankle thickness | | Day 26 | |
|---|---|---|---|---|---|
| | | (mm) ^{a} | | Δ mm | |
| Treatment | | Day 18 | Day 26 | (vs. group 1) | % Inh |
| Group 1: | mean (n = 4) | 7.20 | 7.26 | 0 | 100 |
| Sterile Saline | SD | 0.043 | 0.012 | | |
| Vehicle | p-value ^{b} | 0.0000 | 0.0001 | | |
| Day 18-26 | | | | | |
| Group 2: | mean (n = 5) | 11.86 | 12.31 | 5.04 | 0 |
| PG-PS (15 μg/ | SD | 0.77 | 1.26 | | |
| gram BW) | | | | | |
| Vehicle | p-value * | na | na | | |
| Day 18-26 | | | | | |
| Group 3: | mean (n = 5) | 11.79 | 10.93 | 3.67 | 27 |
| PG-PS (15 μg/ | SD | 0.56 | 0.21 | | |
| gram BW) | | | | | |
| Compound α | p value * | 0.88 | 0.043 | | |
| (5 mg/kg) | | | | | |
| Day 18-26 | | | | | |
| Group 4: | mean (n = 5) | 11.76 | 10.42 | 3.15 | 37 |
| PG-PS (15 μg/ | SD | 0.73 | 0.93 | | |
| gram BW) | | | | | |
| Compound α | p-value * | 0.85 | 0.028 | | |
| (20 mg/kg) | | | | | |
| Day 18-26 | | | | | |
| Group 5: | mean (n = 5) | 11.68 | 9.99 | 2.73 | 46 |
| PG-PS (15 μg/ | SD | 0.62 | 0.73 | | |
| gram BW) | | | | | |
| Compound α | p-value * | 0.71 | 0.0075 | | |
| (50 mg/kg) | | | | | |
| Day 18-26 | | | | | |

^{a} Calipers were used to measure the width (anterior to posterior surface) of the left and right hind ankles of each rat. Each ankle was measured 3 times and averaged.
^{b} Student's t-test vs. group 2

Hind paw inflammation clinical scores were assigned based on swelling and erythema. By day 18, nearly all rats induced with SCW PG-PS had a clinical score of 8 based on an 8-point scale (Table 8). Treatment with Compound α induced a dose dependent decrease in clinical score measurements with significant effects emerging at the 20 mg/kg dose (Table 8).

TABLE 8
Clinical Scores of rats with SCW-arthritis
dosed with vehicle vs. Compound α

| | | Clinical Scores | | |
|---|---|---|---|---|
| | | (0-8) ^{a} | | Δ Day 18 vs. |
| Treatment | | Day 18 | Day 26 | day 26 |
| Group 1: | mean (n = 4) | 0 | 0 | 0 |
| Sterile Saline | SD | 0 | 0 | |
| Vehicle | p-value ^{b} | | <0.0001 | |
| Day 18-26 | | | | |
| Group 2: | mean (n = 5) | 7.80 | 7.80 | 0 |
| PG-PS (15 μg/ | SD | 0.45 | 0.45 | |
| gram BW) | | | | |
| Vehicle | p-value | | na | |
| Day 18-26 | | | | |
| Group 3: | mean (n = 5) | 8.00 | 6.80 | −1.20 |
| PG-PS (15 ug/ | SD | 0.00 | 1.09 | |
| gram BW) | | | | |
| Compound α | p-value | | 0.095 | |
| (5 mg/kg) | | | | |
| Day 18-26 | | | | |
| Group 4: | mean (n = 5) | 8.00 | 5.20 | −2.80 |
| PG-PS (15 μg/ | SD | 0.00 | 1.79 | |
| gram BW) | | | | |
| Compound α | p-value | | 0.014 | |
| (20 mg/kg) | | | | |
| Day 18-26 | | | | |
| Group 5: | mean (n = 5) | 7.80 | 4.40 | −3.40 |
| PG-PS (15 μg/ | SD | 0.45 | 1.67 | |
| gram BW) | | | | |
| Compound α | p-value | | 0.0023 | |
| (50 mg/kg) | | | | |
| Day 18-26 | | | | |

^{a} Hind paw inflammation clinical scores were assigned based on swelling and erythema as follows: 1 = ankle involvement only; 2 = involvement of ankle and proximal ½ of tarsal joint; 3 = involvement of the ankle and entire tarsal joint down to the metatarsal joints; and 4 = involvement of the entire paw including the digits. Scores of both hind-paws were summed for a maximal score of 8.
^{b} Student's t-test vs. group 2

Compound α Reduced Active MMP9 in Ankles of Rats with SCW-Induced Arthritis Demonstrated by Western Blot Analysis

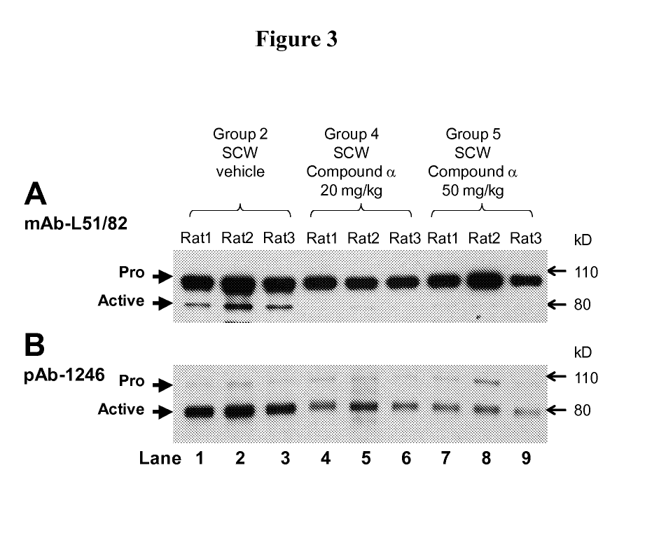

Rats in the study reported in Tables 7 and 8 were sacrificed on day 26 four hours after the AM dose Ankles harvested from the right-hind-limbs were processed by the method described above. Pro and active MMP9 were abundantly present in ankles of SCW-induced vehicle-treated rats (FIGS. 3A and 3B, lanes 1-3). Treatment of rats with Compound α did not reduce the abundance of proMMP9 (FIG. 3A, lanes 4-9). However, treatment of rats with Compound α resulted in a notable reduction in the active 80 kD form of MMP9 detected with pAb-1246 (FIG. 3B, lanes 4-9 vs. 1-3) and with mAb-L51/82 (FIG. 3A, lanes 4-9 vs. 1-3).

Compound α Reduced MMP9 Mediated Gelatinase Activity in the Livers of Rats with SCW Arthritis

In situ zymography provides an alternative approach to assess active MMP9 in tissues (J Histochem Cytochem; 2004; 52:711-722). Tissue sections are overlain with fluorescein-conjugated gelatin wherein the conjugation is sufficiently dense to cause the fluorescein to be dye-quenched (DQ). Proteolytic degradation of the DQ-gelatin releases the fluorescein from the quenching effect giving rise to bright green fluorescence at the site of degradation. Because in situ zymography requires the use of frozen sections, calcified tissues are problematic. However, an additional feature of the SCW arthritis model is the development of hepatic granulomatous disease (J Immunol; 1986; 137:2199-2209), and MMP9 reportedly plays a role in macrophage recruitment in the granulomas response to mycobacteria (Infect Immun; 2006; 74:6135-6144). Consequently, granulomatous livers from SCW-treated rats were assessed for active MMP9 by in situ zymography.

As described above, Female Lewis (LEW/N) rats, 5-6 weeks of age (80-100 g) were injected (i.p.) with saline or SCW PG-PS. On day 28, when the granulomatous response was well established, animals were sacrificed and livers were frozen in OCT cryo-sectioning medium and 10 μm sections were cut on a Cryome HM 500 M cryotome and mounted on glass microscope slides. Sections were air dried briefly. MMP9 was confirmed as the source of the gelatinase activity in the liver by treating liver sections with monoclonal antibodies directed against the active site of the two major gelatinases MMP9 and MMP2. Liver sections overlain with 50 μL of 100 μg/mL neutralizing mouse monoclonal antibodies directed against MMP9 (Calbiochem, clone 6-6B), or MMP2 (Millipore, clone CA-4001), or with PBS for 1 hr at room temperature. Tissues were rinsed once with PBS, blotted, and briefly air dried and then overlain with DQ-gelatin (Invitrogen) dissolved to 1 mg/mL in deionized water and then diluted 1:10 in 1% wt/vol low gelling point agarose type VII (Sigma) in PBS. The sections were covered with coverslips, incubated in the dark at room temperature for 20 min, and imaged on an Olympus IX80 inverted microscope fitted with fluorescence optics, using SlideBook™ imaging software (Intelligent Imaging Innovations, Inc., Philadelphia, Pa.; version 5.0). Fluorescence intensity was determined (Table 9). When compared to a saline-treated rat, gelatinase activity was abundantly expressed in granulomatous liver sections obtained from a rat with SCW arthritis. The activity in the granulomatous liver sections was almost completely inhibited by treatment with anti-MMP9 monoclonal antibody but not by treatment with anti-MMP2 monoclonal antibody.

TABLE 9
Indentification of MMP9 as the gelatinase responsible for signals
detected by in situ zymography in SCW-granulomatous livers

| | |
|---|---|
| Disease | Intensity (RLU × 10⁶) |

| induction | Section treatment | Mean | SD |
|---|---|---|---|
| Saline-healthy | PBS | 11.4 | 2.91 |
| SCW- | PBS | 109 | 19.3 |
| granulomatous | Anti-MMP9 | 1.02 | 0.17 |
| | Anti-MMP2 | 128 | 36.2 |

Key: RLU = relative light units; SCW = Streptococcal cell wall peptidoglycan-polysaccharide equivalent to 15 μg rhamnose/gram BW.

Next, liver in situ zymography was used to assess the relative presence of active MMP9 in rats dosed with vehicle vs. Compound α. Female Lewis (LEW/N) rats, 5-6 weeks of age (80-100 g) were injected (i.p.) with saline or SCW PG-PS. Commencing on day 25, randomized groups of rats (n=3 rats/group) received vehicle or 20 or 50 mg/kg Compound α BID by oral gavage. Vehicle consisted of an aqueous mixture containing 2% (v:v) N-methylpyrrolidone, 5% (v:v) glycerine, and 20% (w:v) captisol. Treatment continued daily through the morning of day 28.

Four hrs after the AM dose on day 28, rats were sacrificed and livers assessed for active MMP9 by in situ zymography (Table 10). Gelatinase activity was increased markedly in SCW-induced rats, but activity was reduced by approximately 80% in animals treated with 50 mg/kg Compound α.

TABLE 10
In situ zymography determination of gelatinase activity in
livers of SCW-induced rats dosed with vehicle vs. Compound α

| | | | | | | t-test vs. |
|---|---|---|---|---|---|---|
| | Intensity (RLU × 10⁶) | | | | | SCW- |
| Treatment | Rat 1 | Rat 2 | Rat 3 | Mean | SD | vehicle |
| Saline | 3.3 | 1.1 | 1.6 | 2.0 | 1.15 | 0.001 |
| Vehicle | | | | | | |
| Day 25-28 | | | | | | |
| SCW | 65.1 | 43.4 | 58.9 | 55.8 | 11.17 | 1 |
| Vehicle | | | | | | |
| Day 25-28 | | | | | | |
| SCW | 43.0 | 69.0 | 53.7 | 55.2 | 13.06 | 0.96 |
| Compound α | | | | | | |
| (20 mg/kg) | | | | | | |
| Day 25-28 | | | | | | |
| SCW | 3.2 | 25.6 | 4.5 | 11.1 | 12.57 | 0.010 |
| Compound α | | | | | | |
| (50 mg/kg) | | | | | | |
| Day 25-28 | | | | | | |

Key: RLU = relative light units; SCW = Streptococcal cell wall peptidoglycan-polysaccharide equivalent to 15 μg rhamnose/gram BW.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

All publications disclosed in the above specification are hereby incorporated by reference in full.

We claim:

1. A method of inhibiting activation of matrix metalloproteinase proMMP9 and/or proMMP13 using a small molecule selected from the group consisting of: 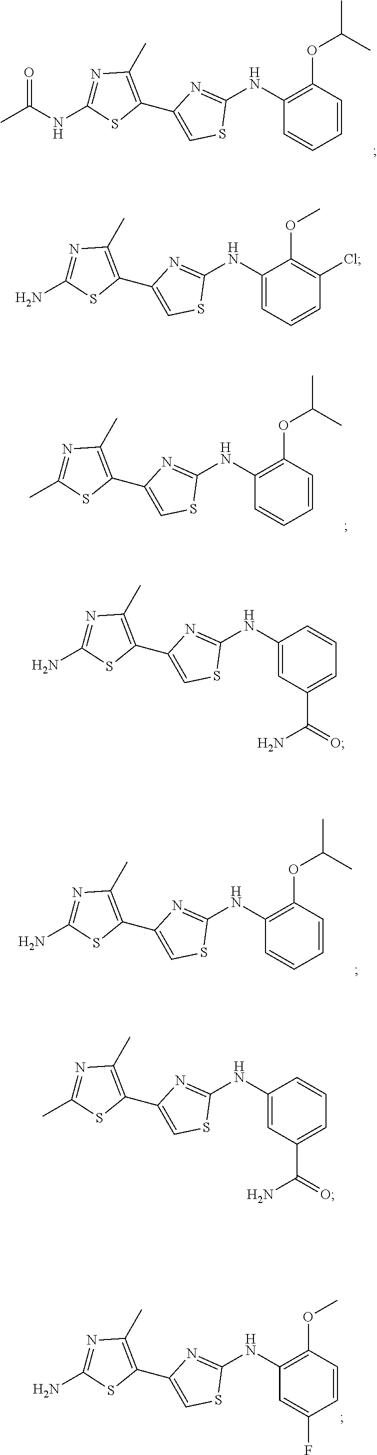 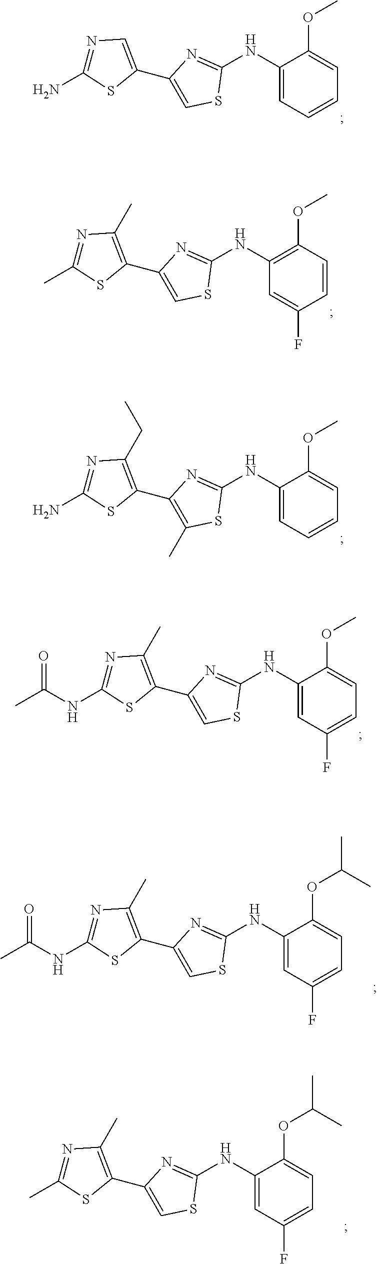 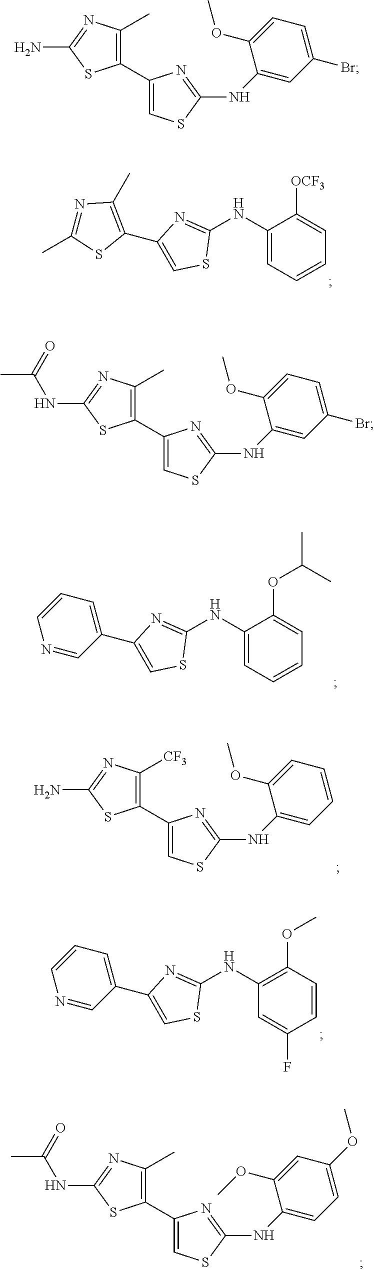 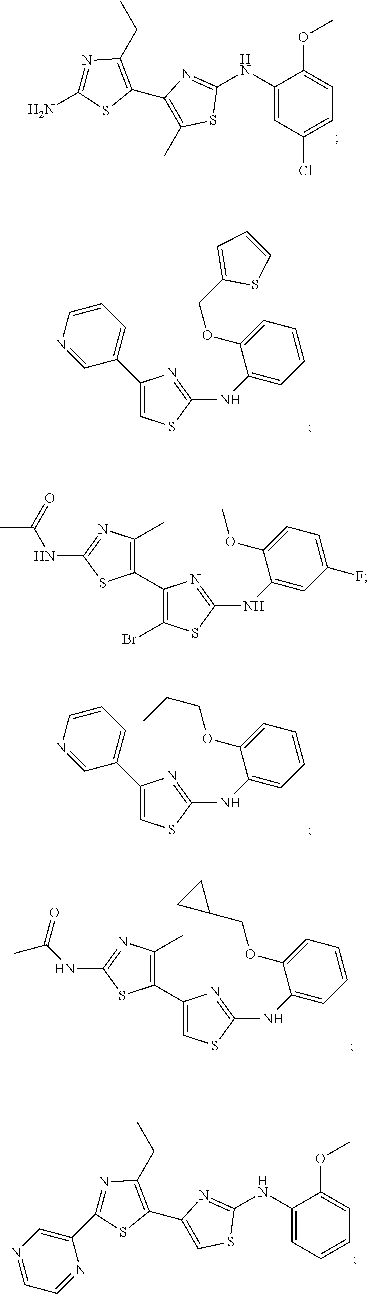 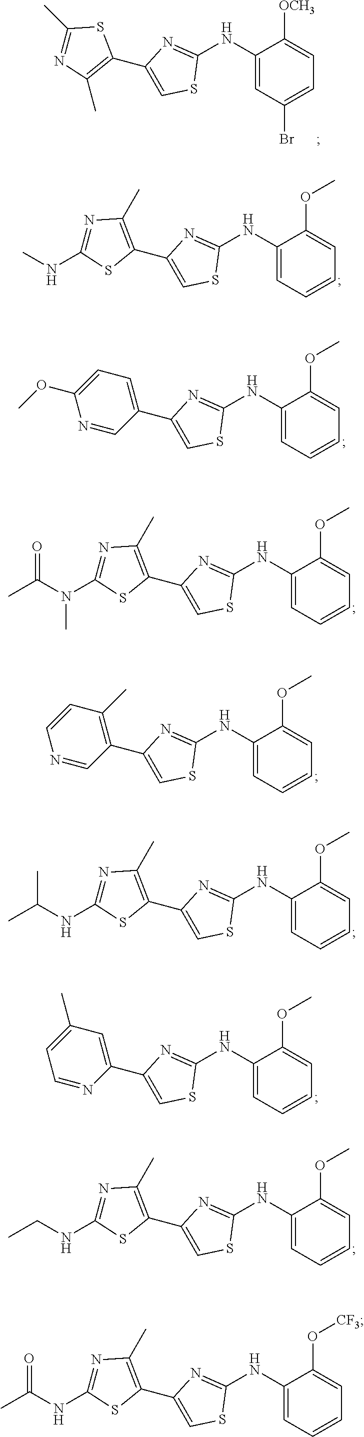
and solvates, hydrates, tautomers, and pharmaceutically acceptable salts thereof.

2. A method of inhibiting activation of matrix metalloproteinase proMMP9 and/or proMMP13 using a pharmaceutical composition, comprising a compound listed in the examples section of this specification and a pharmaceutically acceptable carrier.

3. A method for preventing, treating or ameliorating an MMP9 mediated syndrome, disorder or disease comprising administering to a subject in need thereof an effective amount of a compound listed in the examples section of this specification or a form, composition or medicament thereof.

4. A method for preventing, treating or ameliorating an MMP9 mediated syndrome, disorder or disease wherein said syndrome, disorder or disease is associated with elevated MMP9 expression or MMP9 overexpression, or is a condition that accompanies syndromes, disorders or diseases associated with elevated MMP9 expression or MMP9 overexpression comprising administering to a subject in need thereof an effective amount of a compound listed in the examples section of this specification or a form, composition or medicament thereof.

5. A method of preventing, treating or ameliorating a syndrome, disorder or disease, wherein said syndrome, disorder or disease is selected from the group consisting of: neoplastic disorders, osteoarthritis, rheumatoid arthritis, cardiovascular diseases, gastric ulcer, pulmonary hypertension, chronic obstructive pulmonary disease, inflammatory bowel syndrome, periodontal disease, skin ulcers, liver fibrosis, emphysema, Marfan syndrome, stroke, multiple sclerosis, asthma, abdominal aortic aneurysm, coronary artery disease, idiopathic pulmonary fibrosis, renal fibrosis, and migraine, comprising administering to a subject in need thereof an effective amount of a compound listed in the examples section of this specification or a form, composition or medicament thereof.

6. The method of claim 5, wherein said syndrome, disorder or disease is a neoplastic disorder, which is ovarian cancer.

7. The method of claim 5, wherein said syndrome, disorder or disease is a cardiovascular disease, wherein said cardiovascular disease is selected from the group consisting of: atherosclerotic plaque rupture, aneurysm, vascular tissue morphogenesis, coronary artery disease, and myocardial tissue morphogenesis.

8. The method of claim 7, wherein said cardiovascular disease is atherosclerotic plaque rupture.

9. The method of claim 5, wherein said syndrome, disorder or disease is rheumatoid arthritis.

10. The method of claim 5, wherein said syndrome, disorder or disease is asthma.

11. The method of claim 5, wherein said syndrome, disorder or disease is chronic obstructive pulmonary disease.

12. The method of claim 5, wherein said syndrome, disorder or disease is inflammatory bowel syndrome.

13. The method of claim 5, wherein said syndrome, disorder or disease is abdominal aortic aneurism.

14. The method of claim 5, wherein said syndrome, disorder or disease is osteoarthritis.

15. The method of claim 5, wherein said syndrome, disorder or disease is idiopathic pulmonary fibrosis.

16. A method of inhibiting MMP9 activity in a mammal by administration of an effective amount of at least one compound listed in the examples section of this specification.

17. A method for preventing, treating or ameliorating an MMP13 mediated syndrome, disorder or disease comprising administering to a subject in need thereof an effective amount of a compound listed in the examples section of this specification or a form, composition or medicament thereof.

18. A method for preventing, treating or ameliorating an MMP13 mediated syndrome, disorder or disease wherein said syndrome, disorder or disease is associated with elevated MMP13 expression or MMP13 overexpression, or is a condition that accompanies syndromes, disorders or diseases associated with elevated MMP13 expression or MMP13 overexpression comprising administering to a subject in need thereof an effective amount of a compound listed in the examples section of this specification or a form, composition or medicament thereof.

19. A method of inhibiting MMP13 activity in a mammal by administration of an effective amount of at least one compound listed in the examples section of this specification.
